SEQUENCE LISTING (8 sequences)

SEQ ID NO: 1 - DNA, 52 bp, Artificial Sequence
  Chemically synthesized
ccatctcatccctgcgtgtctccgactcagtcagagtttgatcctggctcag

SEQ ID NO: 2 - DNA, 59 bp, Artificial Sequence
  Chemically synthesized
cctatcccctgtgtgccttggcagtctcagnnnnnnnncatgctgcctcccgtaggagt

SEQ ID NO: 3 - DNA, 18 bp, Artificial Sequence
  Chemically synthesized
ccagccaagtagcgtgca

SEQ ID NO: 4 - DNA, 18 bp, Artificial Sequence
  Chemically synthesized
tggaccttccgtattacc

SEQ ID NO: 5 - DNA, 19 bp, Artificial Sequence
  Chemically synthesized
gcaactctttacgcccagt

SEQ ID NO: 6 - DNA, 18 bp, Artificial Sequence
  Chemically synthesized
gagaggatgatcagccag

SEQ ID NO: 7 - DNA, 18 bp, Artificial Sequence
  Chemically synthesized
agagtttgatcctggctc

SEQ ID NO: 8 - DNA, 19 bp, Artificial Sequence
  Chemically synthesized
tgctgcctcccgtaggagt

(19) United States
(12) Patent Application Publication
(10) Pub. No.: US 2014/0377278 A1
(43) Pub. Date: Dec. 25, 2014

(54) Compositions and Methods for Assessing and Treating Inflammatory Diseases and Disorders

(71) Applicants: Eran Elinav, Woodbridge, CT (US); Richard A. Flavell, Guilford, CT (US); Till Strowig, Braunschweig (DE); Jorge H. Mejia, New Haven, CT (US); Chengcheng Jin, New Haven, CT (US)

(72) Inventors: Eran Elinav, Woodbridge, CT (US); Richard A. Flavell, Guilford, CT (US); Till Strowig, Braunschweig (DE); Jorge H. Mejia, New Haven, CT (US); Chengcheng Jin, New Haven, CT (US)

(73) Assignee: Yale University, New Haven, CT (US)

(21) Appl. No.: 14/343,098

(22) PCT Filed: Apr. 16, 2012
(86) PCT No.: PCT/US12/33753

§ 371 Date: Jul. 23, 2014

Related U.S. Application Data
(60) Provisional application No. 61/584,988, filed on Jan. 10, 2012.

(51) Int. Cl. A61K 35/74; C12Q 1/68; C12N 15/113; C07K 16/28; A61K 45/06; C07K 16/24

(52) U.S. Cl. CPC A61K 35/741; A61K 45/06; C07K 16/24; C12N 15/1136; C07K 16/2866; C12N 15/1138; C12Q 1/6869; A61K 2035/115
USPC 424/158.1; 424/93.45; 514/44 A; 424/172.1; 435/6.12

(57) ABSTRACT

The present invention relates to the discovery that the disruption of inflammasome function leads to an altered microbiota that affects the development and progression of inflammatory diseases and disorders. Thus, the invention relates to compositions and methods for detecting and determining the relative proportions of the constituents of a subject's microbiota, methods of modifying an altered microbiota population in a subject, and compositions and methods for treating inflammatory diseases and disorders in a subject in need thereof.

### BACKGROUND OF THE INVENTION

The distal intestine of humans contains tens of trillions of microbes; this community (microbiota) is dominated by members of the domain Bacteria but also includes members of Archaea and Eukarya and their viruses. The vast repertoire of microbial genes (microbiome) that are present in the distal gut microbiota performs myriad functions that benefit the host (Qin et al., 2010, Nature 464:59-65). The mucosal immune system coevolves with the microbiota beginning at birth, acquiring the capacity to tolerate components of the microbial community while maintaining the capacity to respond to invading pathogens. The gut epithelium and its overlying mucus provide a physical barrier. Epithellal cell lineages, notably the Paneth cell, sense bacterial products through receptors for microbe-associated molecular patterns (MAMPs), resulting in regulated production of bactericidal molecules (Vaishnava et al., 2008, Proc. Natl. Acad. Sci. USA 105:20858-20863). Mononuclear phagocytes continuously survey luminal contents and participate in maintenance of tissue integrity and the initiation of immune responses (Macpherson and Uhr, 2004, Trends Immunol. 25:677-686; Niess et al., 2005, Science 307:254-258; Rescigno et al., 2001, Nat. Immunol. 2:361-367).

Several families of innate receptors expressed by hematopoietic and nonhematopoietic cells are involved in recognition of MAMPs, such as Toll-like receptors (TLRs), nucleotide-binding oligomerization domain protein-like receptors (NLRs), and C-type lectin receptors (Geijtenbeek et al., 2004, Annu. Rev. Immunol. 22:33-54; Janeway and Medzhitov, 2002, Annu. Rev. Immunol, 20:197-216; Martinon et al., 2002, Cell 10:417-426). Inflammasomes are cytoplasmic multiprotein complexes that are composed of one of several NLR proteins, including NLRP1, NLRP3, and NLRC4, which function as sensors of endogenous or exogenous stress or damage-associated molecular patterns (Schroder and Tschopp, 2010, Cell 140:821-832). Upon sensing the relevant signal, they assemble, typically together with the adaptor protein, apoptosis-associated speck-like protein (ASC), into a multiprotein complex that governs caspase-1 activation and subsequent cleavage of effector proinflammatory cytokines, including pro-IL-1b and pro-IL-18 (Agostini et al., 2004, Immunity 20:319-325; Martinon et al., 2002, Cell 10:417-426).

Several other members of the NLR family, including NLRP6 and NLRP12, possess the structural motifs of molecular sensors and are recruited to the “specks” formed in the cytosol by ASC oligomerization, leading to procaspase-1 activation (Grenier et al., 2002, FEBS Lett. 530:73-78; Wang et al., 2002, J. Biol. Chem. 277:29874-29880). However, the triggers and function of NLRP12 are only now being revealed (Arthur et al., 2010, J. Immunol. 185:4515-4519), and those of NLRP6 remain unknown.

Four previous reports indicated that caspase-1, ASC, or NLRP3 deficiencies were associated with an increased severity of acute DSS colitis in mice and suggested that exacerbated disease was mediated, in part, by a defect in repair of the intestinal mucosa (Allen et al., 2010, J. Exp. Med. 207:1045-1056; Dupaul-Chicoine et al., 2010, Immunity 32:367-378; Hirota et al., 2011, Inflamm. Bowel Dis. 17:1359-1372; Zaki et al., 2010, Immunity 32:379-391). Opposing results were found in two other studies using the same colitis model. The first study to investigate the role of caspase-1 in intestinal autoinflammation, even prior to the discovery of the inflammasome, found ameliorated acute and chronic colitis in caspase-1^{-/-} mice (Siegmund et al., 2001, Proc. Natl. Acad. Sci. USA 98:13249-13254). More recently, a second study demonstrated reduced severity of disease in NLRP3^{-/-} mice that correlated with decreased levels of proinflammatory IL-1β (Bauer et al., 2010, Gut 59:1192-1199). It has been hypothesized that these differences might be the result of distinct roles of inflammasomes in nonhematopoietic versus hematopoietic cells (Siegmund, 2010, Immunity 32:300-302). The proposed function in epithelial cells is to regulate secretion of IL-18 that stimulates epithelial cell barrier function and regeneration, whereas in hematopoietic cells, inflammasome activation would have a proinflammatory effect.

Recent studies have highlighted the importance of the gut microbiota in the pathogenesis of various autoimmune disorders that manifest outside of the gastrointestinal tract. In some autoimmune models, germ-free conditions or inoculation with a microbiota from healthy mice ameliorates disease (Lee et al., 2010, Proc. Natl. Acad. Sci. USA 108 Suppl. 1:4615-4622; Mazmanian et al., 2008, Nature 453:620-625; Sinkorová et al., 2008, Hum. Immunol. 69:845-850; Wu et al., 2010, Immunity 32:815-827). In contrast, rats with collagen-induced arthritis feature exacerbated disease when reared under germ-free conditions (Breban et al., 1993, Clin. Exp. Rheumatol. 11:61-64), whereas germ-free NOD MyD88^{-/-} mice fail to develop diabetes, unlike their colonized counterparts (Wen et al., 2008, Nature 455:1109-1113). In humans, epidemiological evidence points to possible links between dysbiosis and rheumatoid arthritis, asthma, and atopic dermatitis (Björkstén, 1999, Allergy 54 (Suppl. 49):17-23; Penders et al., 2007, Allergy 62:1223-1236; Vaahtovuo et al., 2008, J. Rheumatol. 35:1500-1505).

The prevalence of non-alcoholic fatty liver disease (NAFLD) ranges from 20-30% in the general population and up to 75-100% in obese individuals (Sheth et al., 1997, Ann. Intern. Med. 126:137-145; Ludwig et al., 1980, Mayo Clin. Proc. 55:434-438). NAFLD is considered one of the manifestations of metabolic syndrome (Marchesini et al., 2003, Hepatology 37:917-923). Whereas most patients with NAFLD remain asymptomatic, 20% progress to develop chronic hepatic inflammation (non-alcoholic steatohepatitis, NASH), which in turn can lead to cirrhosis, portal hypertension, hepatocellular carcinoma and increased mortality (Caldwell et al., 1999, Hepatology 29:664-669; Shimada et al., 2002, J. Hepatol. 37:154-160; Propst et al., 1995, Gastroenterology 108:1607). Despite its high prevalence, factors leading to progression from NAFLD to NASH remain poorly understood and no treatment has proven effective (Charlton, 2008, Hepatology 47:1431-1433; Hjelkrem et al., 2008, Minerva Med. 99:583-593).

A “two hit” mechanism is proposed to drive NAFLD/NASH pathogenesis (Day et al., 1998, Gastroenterology 114:842-845). The first hit, hepatic steatosis, is closely associated with lipotoxicity-induced mitochondrial abnormalities that sensitize the liver to additional pro-inflammatory insults. These second hits include enhanced lipid peroxidation and increased generation of reactive oxygen species (ROS) (Sanyal et al., 2001, Gastroenterology 120; 1183-1192). Inflammasomes are cytoplasmic multi-protein complexes composed of one of several NLR and PYHIN proteins, including NLRP1, NLRP3, NLRC4 and AIM2. Inflammasomes are sensors of endogenous or exogenous pathogen-associated molecular patterns (PAMPs) or damage-associated molecular patterns (DAMPs) (Sutterwala et al., 2007, J. Leukoc. Biol. 82:259-264) that govern cleavage of effector proinflammatory cytokines such as pro-IL-1b and pro-IL-18 (Martinon et al., 2002, Cell 10:417-426; Agostini et al., 2004, Immunity 20:319-325). Most DAMPs trigger the generation of ROS, which are known to activate the NLRP3 inflammasome (Zhou et al., 2011, Nature 469:221-225).

Recent reports suggest a complex role of inflammasome function in multiple manifestations of the metabolic syndrome. Activation of IL-1β, mainly through cleavage by the NLRP3 inflammasome, promotes insulin resistance (Vandanmagsar et al., 2011, Nature Med. 17:179-188; Wen et al., 2011, Nature Immunol. 12:408-415), atherosclerotic plaque formation (Duewell et al., 2010, Nature 464:1357-1361), and β cell death (Zhou et al., 2010, Nature Immunol. 11:136-140; Masters et al., Nature Immunol. 11:897-904). Moreover, caspase-1 activation seems to direct adipocytes towards a more insulin-resistant phenotype (Stienstra et al., 2011, Proc. Nat. Acad. Sci. USA 108:15324-15329). Conversely, IL18-deficient mice are prone to develop obesity, hyperphagia and insulin resistance (Netea et al., 2006, Nature Med. 12:650-656). These discrepancies most probably reflect a hierarchical contribution of multiple inflammasome components in different metabolic processes, tissues and mouse models. In agreement with previous studies, we found increased obesity and insulin resistance in IL18-deficient mice fed with a HFD. However, and in contrast to two previous reports (Wen et al, 2011, Nature Immunol. 12:408-415; Stienstra et al., 2011, Proc. Nat. Acad. Sci. USA 108:15324-15329), it is herein shown that Asc^{-/-} mice are prone to obesity induction and hepatosteatosis, as well as impaired glucose homeostasis when fed a HFD. Alterations in intestinal microbiota communities associated with multiple inflammasome deficiencies could account for these discrepancies and it should be added to the list of major environmental/host factors affecting manifestations and progression of metabolic syndrome in susceptible populations.

There is thus a need in the art for compositions and methods for assessing and treating inflammatory disorders associated with an altered microbiota. The present invention addresses these unmet needs in the art.

### SUMMARY OF THE INVENTION

The invention relates to the discovery that the disruption of inflammasome function leads to an altered microbiota that affects the development and progression of inflammatory diseases and disorders. Thus, the invention relates to compositions and methods for detecting and determining the relative proportions of the constituents of a subject's microbiota, methods of modifying an altered microbiota population in a subject, and compositions and methods for treating inflammatory diseases and disorders in a subject in need thereof.

In one embodiment, the invention is a method of diagnosing an altered microbiota associated with an inflammatory disease or disorder in a subject in need thereof, including the steps of: obtaining a fecal sample from a subject, obtaining bacterial nucleic acid from the fecal sample, amplifying the bacterial nucleic acid using PCR, sequencing the amplicons resulting from the amplification of the bacterial nucleic acid using PCR, identifying the types of bacteria present in the biological sample obtained from the subject by detecting nucleic acid sequences that are specific to particular types of bacteria, quantifying the types of bacteria present in the biological sample obtained from the subject by quantifying nucleic acid sequences that are specific to particular types of bacteria, determining the relative proportions of the types of bacteria present in the fecal sample obtained from the subject, comparing the relative proportions of the types of bacteria present in the fecal sample obtained from the subject with the relative proportions of the types of bacteria present in a normal microbiota, wherein when at least one Lactobacillus spp. is under-represented in the biological sample obtained from the subject, as compared with a normal microbiota, and wherein when at least one type of bacteria selected from the group consisting of Prevotellaceae, TM7, Porphyromonadaceae, and Erysipelotrichaceae is over-represented in the biological sample obtained from the subject, as compared with a normal microbiota, the subject is diagnosed with an altered microbiota associated with an inflammatory disease or disorder. In some embodiments, the bacterial nucleic acid is 16S rRNA. In various embodiments, the inflammatory disease or disorder is at least one of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH). In preferred embodiments, the subject is human.

In another embodiment, the invention is a method of treating an inflammatory disease or disorder associated with an altered microbiota in a subject in need thereof, by modifying the altered microbiota to that of a normal microbiota, including the steps of: administering to the subject at least one type of bacteria that is under-represented in the altered microbiota of the subject, and administering to the subject at least one antibiotic to diminish the numbers of at least one type of bacteria that is overrepresented in the altered microbiota. In some embodiments, the at least one type of bacteria that is under-represented in the altered microbiota is at least one Lactobacillus spp. In some embodiments, at least one Lactobacillus spp. is administered to the subject. In some embodiments, the at least one type of bacteria that is overrepresented in the altered microbiota is at least one of Prevotellaceae, TM7, Porphyromonadaceae, and Erysipelotrichaceae. In various embodiments, the inflammatory disease or disorder is at least one of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH). In preferred embodiments, the subject is human.

In a further embodiment, the invention is a method of treating an inflammatory disease or disorder associated with an altered microbiota in a subject in need thereof, including the step of: administering to the subject a therapeutically effective amount of a composition comprising a CCL5 inhibitor. In one embodiment, the CCL5 inhibitor is an antibody that specifically binds to CCL5. In various embodiments, the antibody is at least one of: a polyclonal antibody, a monoclonal antibody, an intracellular antibodies, an antibody fragment, a single chain antibody (scFv), a heavy chain antibody, a synthetic antibody, a chimeric antibody, and humanized antibody. In another embodiment, the CCL5 inhibitor is an antisense nucleic acid. In various embodiments, the antisense nucleic acid is at least one of siRNA or miRNA. In other various embodiments, the CCL5 inhibitor is at least one of: a chemical compound, a protein, a peptide, a peptidomemetic, a ribozyme, and a small molecule chemical compound. In various embodiments, the inflammatory disease or disorder is at least one of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH). In preferred embodiments, the subject is human.

In yet another embodiment, the invention is a method of treating an inflammatory disease or disorder associated with an altered microbiota in a subject in need thereof, including the step of: administering to the subject a therapeutically effective amount of a composition comprising a CCL5 receptor inhibitor. In various embodiments the CCL5 receptor inhibitor is at least one of: CCR1, CCR3, CCR4, CCR5 and GPR75. In one embodiment, the CCL5 receptor inhibitor is an antibody that specifically binds to a CCL5 receptor. In various embodiments, the antibody is at least one of: a polyclonal antibody, a monoclonal antibody, an intracellular antibodies, an antibody fragment, a single chain antibody (scFv), a heavy chain antibody, a synthetic antibody, a chimeric antibody, and humanized antibody. In another embodiment, the CCL5 receptor inhibitor is an antisense nucleic acid. In various embodiments, the antisense nucleic acid is at least one of siRNA or miRNA. In other various embodiments, the CCL5 inhibitor is at least one of: a chemical compound, a protein, a peptide, a peptidomemetic, a ribozyme, and a small molecule chemical compound. In various embodiments, the inflammatory disease or disorder is at least one of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH). In preferred embodiments, the subject is human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A-1H, depicts how the increased severity of colitis in ASC-deficient mice is transmissible to cohoused wild-type mice. FIG. 1A is a graph depicting the weight loss of ASC^{-/-} mice and separately housed wild-type (WT) mice. FIG. 1B is a graph depicting the weight loss of ASC^{-/-} mice and WT mice, which were cohoused for 4 weeks, after which DSS colitis was induced. To induce colitis, mice were given 2% DSS in their drinking water for 7 days. FIGS. 1C, 1D, and 1F are graphs depicting weight loss (FIG. 1C), the colonoscopy severity score at day 7 (FIG. 1D), and survival (FIG. 1F) after induction of DSS colitis of WT mice that were cohoused with (i) in-house WT mice bred for several generations in our vivarium (IH-WT) or (ii) ASC^{-/-} mice (designated WT(IH-WT) and WT(ASC^{-/-}), respectively). FIG. 1E is a series of representative images taken during colonoscopy of mice at day 7. (G and H) FIG. 1G is a series of representative H&E-stained sections of colons from WT(IH-WT), WT(ASC^{-/-}), and ASC^{-/-}(WT) mice sampled on day 6 after the start of DSS exposure. FIG. 1H is series of representative H&E-stained sections of colons from WT(IH-WT), WT(ASC^{-/-}), and ASC^{-/-}(WT) mice sampled on day 12 after the start of DSS exposure. Epithelial ulceration (arrowheads), severe edema/inflammation (asterisk) with large lymphoid nodules (L), retention/regeneration of crypts (arrows), and evidence of re-epithelialization/repair of the epithelium (box). Scale bars, 500 mm. Data are representative for three independent experiments. Error bars represent the SEM of samples within a group. *p<0.05 by one-way ANOVA. For related data, see FIGS. 8A-8D.

FIG. 2, comprising FIGS. 2A-2F, depicts maternal transmission of an exacerbated DSS colitis phenotype. FIG. 2A is a graph depicting the measured body weight of ASC^{-/-} mice and ASC^{-/-} mice cross-fostered with WT mothers (CF-ASC^{-/-}). FIG. 2B is a graph depicting the colonoscopy severity score measured in ASC^{-/-} mice and ASC^{-/-} mice cross-fostered with WT mothers (CF-ASC^{-/-}). FIG. 2C is a graph depicting the measured body weight of WT mice and WT mice cross-fostered with ASC^{-/-} mothers. FIG. 2D is a graph depicting the colonoscopy severity score measured in WT mice and WT mice cross-fostered with ASC^{-/-} mothers. (CF-WT). FIG. 2E is a graph depicting the measured body weight of WT mice cohoused with ASC^{-/-} or cross-fostered ASC^{-/-} mice for 4 weeks. FIG. 2F is a graph depicting the colonoscopy severity score measured in WT mice cohoused with ASC^{-/-} or cross-fostered ASC^{-/-} mice for 4 weeks. Data are representative of three independent experiments. Error bars represent the SEM of samples within a group. *p<0.05 by one-way ANOVA. FIGS. 8E-8G contain related data. Newborn ASC^{-/-} and WT mice were swapped between their respective mothers (cross-fostered), followed by induction of acute DSS colitis at 8 weeks of age.

FIG. 3, comprising FIGS. 3A-3F, depicts bacterial 16S rRNA-based analysis of the fecal microbiota of WT and NLRP6 inflammasome-deficient mice. FIG. 3A is a graph depicting the unweighted UniFrac principal coordinates analysis (PcoA) of fecal microbiota harvested from WT mice single-housed or cohoused with ASC^{-/-} mice. FIG. 3B is a graph depicting the unweighted UniFrac PCoA of fecal microbiota harvested from WT mice single-housed or cohoused with IL-18^{-/-} mice. FIG. 3C is a graph depicting the unweighted UniFrac PCoA of fecal microbiota harvested from WT mice single-housed or cohoused with NLRP6^{-/-} mice. FIG. 3D is a graph depicting the unweighted UniFrac PCoA of fecal microbiota harvested from all mice. Samples from mice shown in FIG. 3A and FIG. 3C were taken just prior to cohousing and 28 days later. Dashed line illustrates separation of samples along PC1. FIG. 3E is a graph depicting the distribution of family-level phylotypes in ASC-, IL-18-, NLRP6-deficient, and cohoused WT mice, compared to single-housed WT mice. The horizontal axis shows the fold representation (defined as the ratio of the percentage of samples with genera present in knockout or cohoused mice versus single-housed WT mice). The left side of the axis indicates taxa whose representation is greater in single-housed WT mice; the right denotes taxa whose representation is greater in knockout or cohoused WT mice. The origin represents equivalent recovery of taxa in both groups. The vertical axis shows the calculated p value for each taxa as defined by G test. Open diamonds represent taxa that were found only in KO/cohoused WT or single-housed WT mice but where recovery was assumed to be 1 to calculate fold representation. FIG. 3F is a graph depicting the unweighted UniFrac PCoA demonstrating presence or absence of TM7 and Prevotellaceae in each sample. Dashed lines show separation of single-housed WT and cohoused WT and knockout mice on PC1, PC2 in FIG. 3D and FIG. 3F shows separation of communities based on host genotype/cohousing. For additional data related to the transmission of fecal microbiota in inflammasome deficient mice, see FIG. 9.

FIG. 4, comprising FIGS. 4A-4J, depicts how NLRP6-deficient mice harbor a transmissible colitogenic gut microbiota. FIG. 4A is a graph depicting the analysis of NLRP6 expression in various organs. FIG. 4B is a graph depicting the analysis of NLRP6 expression in colonic epithelial and hematopoietic (CD45⁺) cells. The purity of the sorted populations was analyzed using vil1 and ptprc as markers for epithelial and hematopoietic cells, respectively. FIG. 4C is a graph depicting the analysis of bone marrow chimeras, which were generated using WT and NLRP6^{-/-} mice as host and bone marrow donor, NLRP6 expression in the colon was analyzed 8 weeks after bone marrow transplantation. FIG. 4D is an image of an immunoprecipitation analysis of NLRP6 protein expression using an NLRP6 antibody and lysates of primary colonic epithelial cells isolated from WT and NLRP6^{-/-} mice. FIG. 4E is a representative confocal image of colonic sections analyzed for expression of NLRP6 (red) and counterstained with DAPI at 40× resolution. FIG. 4F is a representative confocal image of colonic sections analyzed for expression of NLRP6 (red) and counterstained with DAPI at 100× resolution. White dotted lines were drawn to illustrate the epithelial cell boundaries. FIGS. 4G-4I are graphs depicting the weight loss (FIG. 4G), colonoscopy severity score at day 8 (FIG. 4H), and survival (FIG. 4I) of single-housed versus cohoused WT and NLRP6^{-/-} mice. Acute DSS colitis was induced in single-housed WT mice, in WT mice cohoused for 4 weeks with NLRP6^{-/-} mice (WT(NLRP6^{-/-})), the corresponding cohoused NLRP6^{-/-} mice (NLRP6^{-/-}(WT)), and single-housed NLRP6^{-/-} mice (NLRP6^{-/-}). FIG. 4J is a series of representative images of H&E-stained sections of colons on day 7 after initiation of DSS exposure. Edema/inflammation (asterisks), ulceration (arrowheads), and loss of crypts (arrow). Scale bars, 500 mm. Data are representative of three independent experiments. Error bars represent the SEM of samples within a group. *p<0.05 by one-way ANOVA. Related data are in FIG. 10 and FIG. 35.

FIG. 5, comprising FIGS. 5A-5L, depicts how processing of IL-18 by NLRP6 inflammasome suppresses colitogenic microbiota. In FIGS. 5A-5C, WT mice were cohoused with IL-1β^{-/-} mice or IL-18^{-/-} mice for 4 weeks, and colitis was subsequently induced with DSS. FIG. 5A is a graph depicting the comparison of weight loss in single-housed WT mice and in WT mice previously cohoused with IL-1β^{-/-} mice (WT(IL-1β^{-/-})). FIG. 5B is a graph depicting weight loss for single-housed WT mice and WT mice previously cohoused with IL-18^{-/-} mice (WT(IL-18^{-/-})). FIG. 5C is a graph depicting the measured colonoscopy severity score at day 7 for single-housed WT mice and WT mice previously cohoused with IL-18^{-/-} mice (WT(IL-18^{-/-})). FIG. 5D is a series of representative images of H&E-stained sections from single-housed WT mice and WT mice cohoused with IL18^{-/-} mice sampled 6 days after the start of DSS administration. Scale bars, 500 mm. FIG. 5E is a graph depicting the inflamed colon area from single-housed WT mice and WT mice cohoused with IL18^{-/-} mice sampled 6 days after the start of DSS administration. FIG. 5F is a graph depicting the pathologic quantitation of colitis severity (from single-housed WT mice and WT mice cohoused with IL18^{-/-} mice sampled 6 days after the start of DSS administration, FIG. 5G is a graph depicting IL-18 levels measured in sera obtained from WT and NLRP6-deficient mice without treatment. FIG. 5H is a graph depicting IL-18 levels measured in colon explants obtained from WT and NLRP6-deficient mice without treatment. FIG. 5I is a graph depicting an analysis of bone marrow chimeras, which were generated using both WT and NLRP6^{-/-} mice as host and bone marrow donor. IL-18 production by colon explants was analyzed 8 weeks after bone marrow transplantation. FIG. 5J is a graph depicting IL-18 concentrations in the serum 5 days after induction of DSS colitis. FIG. 5K is a graph depicting the measured weight at day 7 of mice with acute DSS colitis. FIG. 5L is a graph depicting the measured colonoscopy severity scores at day 7 of mice with acute DSS colitis. Bone marrow chimeras were generated using WT and IL-18^{-/-} mice as host and bone marrow donor. Data in FIGS. 5A-5E are representative of at least three experiments; data in FIGS. 5I-5L) are representative of two experiments, n=6 mice/samples analyzed per group. *p<0.05 by one-way ANOVA. Related data are presented in FIG. 11.

FIG. 6, comprising FIGS. 6A-6I, depicts microbiota induction of CCL5. FIG. 6A is a series of representative images of H&E-stained sections of the colon, terminal ileum, and Peyer's patches from WT, ASC^{-/-}, and NLRP6^{-/-} mice not exposed to DSS. Mucosal hyperplasia in the colon (double arrows), increased crypt to villus ratio in the terminal ileum (asterisks), and enlargement of Peyer's patches with formation of germinal centers (arrowheads). Scale bars, 500 mm. FIG. 6B is a graph depicting the enumeration of subsets of hematopoietic cells harvested from the lamina propria of WT and NLRP6^{-/-} mice. FIG. 6C is a graph depicting the analysis of CCL5 colonic mRNA expression in WT, ASC^{-/-}, NLRP6^{-/-}, and IL-18^{-/-} mice. FIG. 6D is a graph depicting protein expression in colonic explants in WT, ASC^{-/-}, NLRP6^{-/-}, and IL-18^{-/-} mice. FIG. 6E is a graph depicting CCL5 expression in epithelial cells from the colons of WT and NLRP6^{-/-} mice. FIG. 6F is a graph depicting the analysis of CCL5 colonic mRNA expression in single-housed WT mice and WT mice cohoused with NLRP6^{-/-} mice. FIG. 6G is a graph depicting protein expression in colonic explants in single-housed WT mice and WT mice cohoused with NLRP6^{-/-} mice. FIG. 6H is a graph depicting weight loss of mice after induction of acute DSS colitis. FIG. 6I is a graph depicting the measured colonoscopy severity score at day 7 of mice after induction of acute DSS colitis. WT and CCL5^{-/-} mice were either single-housed or cohoused for 4 weeks with NLRP6^{-/-} mice followed by exposure to DSS. Data shown in FIGS. 6A-6G are representative of at least two experiments. Data presented in FIG. 6H and FIG. 6I are from three experiments, n=5-6 mice. Error bars represent the SEM of samples within a group. *p<0.05 by one-way ANOVA. Additional cytokine and chemokine analyses are presented in FIG. 12.

FIG. 7, comprising FIGS. 7A-7L, depicts how decreased abundance of Prevotella in antibiotic-treated NLRP6^{-/-} correlates with ameliorated colitogenic microbiota, FIG. 7A is a graph depicting a comparison of Prevotellaceae loads to total bacteria, which were measured in fecal samples at the end of the antibiotic treatment period using qPCR analysis. FIG. 7B is a graph depicting measured weight loss in WT and NLRP6^{-/-} mice treated with a combination of metronidazole and ciprofloxacin for 3 weeks. FIG. 7C is a graph depicting measured colonoscopy score at day 7 in WT and NLRP6^{-/-} mice treated with a combination of metronidazole and ciprofloxacin for 3 weeks. DSS exposure was begun 3 days later. In FIGS. 7D-7F, NLRP6^{-/-} mice were treated with a combination of ampicillin, neomycin, vancomycin, and metronidazole for 3 weeks and then cohoused with WT mice for 4 weeks. In parallel, WT mice were cohoused with untreated NLRP6^{-/-} mice. Subsequently, DSS colitis was induced. FIG. 7D is a graph depicting the recorded weight loss. FIG. 7E is a graph depicting the measured colonoscopic assessments of mucosal damage at day 7. FIG. 7F is a graph quantitating the results of a qPCR assay for the abundance of Prevotella in fecal samples obtained after 4 weeks of cohousing. FIG. 7G is a graph depicting the unweighted UniFrac PCoA of fecal microbiota harvested after cohousing. FIG. 7H is a graph depicting the unweighted UniFrac PCoA colored by relative abundance of Prevotellaceae as percent of total OTUs. WT mice were cohoused for 4 weeks with either NLRP6^{-/-} or NLRC4^{-/-} mice. FIG. 7I is a graph quantifying Prevotellaceae in the crypt compartment, following extensive removal of stool content. FIG. 7J is a series of representative transmission electron microscopy images taken from colonic sections of WT (×4200) NLRC4^{-/-} (×4200), NLRP6^{-/-} (×2500), and ASC^{-/-} mice (×1700). FIG. 7K is a representative transmission electron microscopy image taken from colonic sections of ASC^{-/-} mice (×4200). FIG. 7L is a representative transmission electron microscopy image taken from colonic sections of ASC^{-/-} mice (×26,000). See FIG. 13 for additional evidence linking bacterial components of the gut microbiota to the transmissible colonic inflammation in NLRP6 inflammasome-deficient mice.

FIG. 8, comprising FIGS. 8A-8G, depicts increased tissue damage in ASC^{-/-} mice and in WT mice cohoused with ASC^{-/-} mice. FIG. 8A is a graph quantifying the inflamed colon area in WT mice in comparison with WT and ASC^{-/-} mice cohoused with each other ((WT(ASC^{-/-}) and ASC^{-/-} (WT), respectively)). FIG. 8B is a graph quantifying the histological severity of DSS colitis in WT mice in comparison with WT and ASC^{-/-} mice cohoused with each other ((WT(ASC^{-/-}) and ASC^{-/-}(WT), respectively)). FIG. 8C is a graph quantifying the inflamed colon area in WT mice in comparison with in-house bred WT mice (IH-WT), and WT and ASC^{-/-} mice cohoused with each other ((WT(ASC^{-/-}) and ASC^{-/-}(WT). FIG. 8D is a graph quantifying the histological severity of DSS colitis in WT mice are compared to in-house bred WT mice (IH-WT), and WT and ASC^{-/-} mice cohoused with each other ((WT(ASC^{-/-}) and ASC^{-/-}(WT), respectively)). In FIGS. 8A-8D quantification of the histological severity of DSS colitis in single-housed WT mice, cohoused WT mice, and ASC^{-/-} mice was determined on day 6 (FIGS. 8A and 8B) and on day 12 (FIGS. 8C and 8D) after initiation of treatment. Results were calculated as the percentage of inflamed colon area (FIGS. 8A and 8C), and the pathological colitis severity scored in the worst affected area (FIGS. 8B and 8D), as quantified by the parameters inflammation, edema, ulceration, hyperplasia and crypt loss. Each parameter was scored by a pathologist, who was blinded to genotype or treatment, between 0 (normal) to 5 (severe). In FIGS. 8E-8G, WT mice were cohoused or not cohoused in two steps to evaluate the stability of the altered flora in WT mice. They were either never cohoused (1st none, 2nd none), cohoused for 4 weeks with ASC^{-/-} mice and then housed separately for 4 weeks (1st ASC^{-/-}, 2nd none), or after their cohousing with ASC^{-/-} mice cohoused with a new cohort of WT mice (1st ASC^{-/-}, 2nd WT). DSS colitis was induced subsequently in these mice. FIG. 8E is a graph depicting weight loss in the mice. FIG. 8F is a graph depicting the measured colonoscopy score at day 7. FIG. 8G is a graph depicting mouse survival. * denotes significance of p<0.05 by One-way ANOVA.

FIG. 9, comprising FIGS. 9A-9G, depicts how exacerbated colitis severity in caspase-1-deficent mice is transmissible to cohoused wild-type mice. In FIGS. 9A-9E, DSS colitis was induced in single-housed WT mice as well as in WT mice cohoused with Casp1^{-/-} mice (WT(Casp1^{-/-}) and Casp1^{-/-}(WT), respectively). FIG. 9A is a graph depicting weight loss in mice. FIG. 9B is a graph depicting the measured colonoscopy score at day 7. FIG. 9C is a series of representative hematoxylin and eosin-stained sections of colons from single-housed WT mice, WT mice cohoused with Casp1^{-/-} mice, and Casp1^{-/-} mice cohoused with WT mice. Colons from Casp1^{-/-} mice and WT mice cohoused with Casp1^{-/-} mice both feature severe pathologic changes, as evidenced by marked epithelial ulceration (arrowheads), loss of crypts, and severe edema (e)/inflammation (*) and flooding of the lumen with inflammatory cells (**). In contrast, colons from WT single house mice had smaller and fewer foci of ulceration (arrowhead), with retention/regeneration of crypts (arrows). Scale bars=500 mm. FIG. 9D is a graph depicting the percentage of inflamed colon area. FIG. 9E is a graph depicting the pathological colitis severity scored in the worst affected area, as quantified by the parameters inflammation, edema, ulceration, hyperplasia and crypt loss. Each parameter was scored by a blinded pathologist between 0 (normal) to 5 (severe). FIG. 9F is a graph quantifying unweighted UniFrac PCoA of fecal microbiota harvested from untreated WT mice single-housed or co-housed with Casp1^{-/-} mice. FIG. 9G is a graph quantifying unweighted UniFrac PCoA demonstrating presence or absence of TM7 and Prevotellaceae in each sample (color key provided at the lower right of the panel). Dashed lines show separation of single-housed WT and co-housed WT and knockout mice on PC1. * represents significance of p<0.05 by One-way ANOVA.

FIG. 10, comprising FIGS. 10A-10H, depicts the increased tissue damage in NLRP6^{-/-} mice and in WT mice cohoused with NLRP6^{-/-} mice. FIG. 10A is an illustration of the generation of NLRP6-deficient mice by replacing Exons 1 and 2 with a neomycin resistance cassette resulting in a truncated gene that lacks the ATG and the coding region for the pyrin domain. FIG. 10B is an image of a gel from a PCR screening strategy for deletion of NLRP6. WT allele 296 bp, targeted allele 524 bp. FIG. 100C is a series of representative images of the colonic mucosa taken during colonoscopy on day 8 of DSS colitis. FIG. 10D is a series of images of representative hematoxylin and eosin-stained sections of colons from WT (WT), WT(NLRP6^{-/-}) and NLRP6^{-/-}(WT) mice sampled on day 6 after induction of DSS colitis. The predominant early differences are evidenced by marked epithelial ulceration (arrowheads), greater loss of crypts, and edema not only within the submucosa (**), but also within the lamina propria (*). In contrast, colons from WT single house mice had smaller and significantly fewer foci of ulceration (arrowhead), with retention of crypts (arrows). Scale bars=500 mm. FIG. 10E-10H depict the quantification of the histological colitis severity in single-housed WT mice (WT), in WT mice cohoused for 4 weeks with NLRP6^{-/-} mice (WT(NLRP6^{-/-})), the corresponding co-housed NLRP6^{-/-} mice (NLRP6−/−(WT)) and single-housed NLRP6^{-/-} mice (NLRP6^{-/-}). FIG. 10E is a graph depicting the percentage of inflamed colon area on day 6 after induction of DSS colitis. FIG. 10F is a graph depicting the pathological colitis severity on day 6 after induction of DSS colitis. FIG. 10G is a graph depicting the percentage of inflamed colon area on day 8 after induction of DSS colitis. FIG. 10H is a graph depicting the pathological colitis severity on day 8 after induction of DSS colitis. Results were calculated as the percentage of inflamed colon area (E, G), and the pathological colitis severity (F, H), as quantified by the parameters inflammation, edema, ulceration, hyperplasia and crypt loss. Each parameter was scored by a blinded pathologist between 0 (normal) to 5 (severe); * represents significance of p<0.05 by One-way ANOVA.

FIG. 11, comprising FIGS. 11A-11K, depicts how other NLR-deficient and inflammasome-associated mouse strains do not house a colitogenic transmissible microbiota. FIG. 11A is a graph quantifying qRT-PCR analysis of the expression of inflammasome-associated genes as well as several NLR genes in total colonic RNA: n.d.=not detectable. FIG. 11B is a graph depicting a comparison of weight loss between single-housed and cohoused mice (WT with AIM2^{-/-} mice). FIG. 11C is a graph depicting a comparison of the measured colonoscopy score between single-housed and cohoused mice (WT with AIM2^{-/-} mice). FIG. 11D is a graph depicting a comparison of weight loss between single-housed and cohoused mice (WT with NLRC4^{-/-} mice). FIG. 11E is a graph depicting a comparison of the measured colonoscopy score between single-housed and cohoused mice (WT with NLRC4^{-/-} mice). FIG. 11F is a graph depicting a comparison of weight loss between single-housed and cohoused mice (WT with NLRP10^{-/-} mice). FIG. 11F is a graph depicting a comparison of the measured colonoscopy score between single-housed and cohoused mice (WT with NLRP10^{-/-} mice). FIG. 11H is a graph depicting a comparison of weight loss between single-housed and cohoused mice (WT with NLRP12^{-/-} mice). FIG. 11I is a graph depicting a comparison of the measured colonoscopy score between single-housed and cohoused mice (WT with NLRP12^{-/-} mice). FIG. 11J is a graph depicting a comparison of weight loss between single-housed and cohoused mice (WT with IL-1R^{-/-} mice). FIG. 11K is a graph depicting a comparison of the measured colonoscopy score between single-housed and cohoused mice (WT with IL-1R^{-/-} mice).

FIG. 12, comprising FIGS. 12A-12K, depicts how CCL5 is essential for the development of exacerbated colitis in cohoused WT mice. FIG. 12A is a graph quantifying colon crypt thickness in ASC^{-/-} and NLRP6^{-/-} mice compared to WT mice. FIG. 12B is a graph quantifying the ileum crypt/vilus ratio in ASC^{-/-} and NLRP6^{-/-} mice compared to WT mice. FIG. 12C is a graph quantifying total levels of IgA measured in the serum of single-housed and cohoused WT and ASC^{-/-} mice. FIG. 12D is a graph quantifying total levels of IgG2C measured in the serum of single-housed and cohoused WT and ASC^{-/-} mice. FIG. 12E is a graph quantifying total levels of IgA measured in the serum of single-housed and cohoused WT NLRP6^{-/-} mice. FIG. 12F is a graph quantifying total levels of IgG2C measured in the serum of single-housed and cohoused WT NLRP6^{-/-} mice. Data are pooled data from 2 independent experiments. FIG. 12G is a graph depicting the multiplex analysis of cytokine and chemokine production in colon tissue explants. FIG. 12H is a graph quantifying total lamina propria immune cells (CD45⁺) and immune cell subsets (Dendritic cells, B cells, αβ T cells, and γδ T cells) in WT and CCL5^{-/-} mice in the steady state. FIG. 12I is a graph depicting unweighted UniFrac PCoA of fecal microbiota harvested from untreated WT and CCL5^{-/-} mice single-housed or co-housed with NLRP6^{-/-} mice. FIG. 12J is a graph depicting unweighted UniFrac PCoA demonstrating presence or absence of TM7 and Prevotellaceae in each sample (color key provided at the lower left of the panel). Dashed lines show separation of single-housed and co-housed WT and knockout mice on PC1, FIG. 12K is a graph quantifying Prevotellaceae 16S rDNA copy numbers normalized to total bacteria. * represents p<0.05 by One-way ANOVA (ns=non significant).

FIG. 13, comprising FIGS. 13A-13H, depicts how the more severe DSS-induced colitis in ASC^{-/-}-deficient mice compared to WT mice is ameloriated with broad-spectrum antibacterial treatment, but not treatment with amphotericin b, gancyclovir, or albendazole and praziquantel. In FIGS. 13A-13B, WT and ASC^{-/-} mice were treated orally with metronidazole/ciprofloxacin for 3 weeks and colitis was induced subsequently. FIG. 13A is a graph depicting changes in body weight. FIG. 13B is a graph depicting the measured colonoscopy score. FIG. 13C is a graph depicting changes in body weight for ASC^{-/-} mice treated for the indicated time intervals with amphotericin B (anti-fungal, 3 weeks). FIG. 13D is a graph depicting changes in body weight for ASC^{-/-} mice treated for the indicated time intervals with albendazole and praziquantel (anti-parasitic, 2 weeks). FIG. 13E is a graph depicting changes in body weight for ASC^{-/-} mice treated for the indicated time intervals with gancyclovir (anti-herpesvirus, 2 weeks). In FIGS. 13C-13E, colitis was subsequently induced and weight loss was compared. Error bars represent the SEM of samples within a treatment group. FIG. 13F is a series of images of representative sections of terminal ileum stained with hematoxylin and eosin (HE), Gram, Geimsa (GMS), and Warthin-Starry (WS) stains to reveal numerous long striated, Gram-negative, GMS-negative, WS-positive rod-shaped bacteria in ASC^{-/-} mice and few in WT mice. Scale bars—50 mm. In FIGS. 13G-13H, NLRP6−/− mice were treated with a combination of ampicillin, neomycin, vancomycin, and metronidazole for 3 weeks and then co-housed with WT mice for 4 weeks. In parallel, WT mice were co-housed with untreated NLRP6^{-/-} mice. FIG. 13G is a graph quantifying a qPCR assay for the abundance of TM7 in fecal samples obtained after 4 weeks of cohousing. FIG. 13H is a graph quantifying a qPCR assay for the abundance of Bacteroides in fecal samples obtained after 4 weeks of cohousing.

FIG. 14, comprising FIGS. 14A-14H, depicts the increased severity of NASH in inflammasome-deficient mice. To induce NASH, mice were fed with MCDD for 24 days. Their serum ALT and AST activities were measured and NAFLD histological activity scores were determined. FIG. 14A is a graph depicting a comparison of ALT and AST activities between singly housed wildtype (WT) mice and Casp1^{-/-} mice. FIG. 14B is a graph depicting a comparison of NAFLD activity plus histological scores for steatosis and inflammation between singly housed WT mice and Casp1^{-/-} mice. FIG. 14C is a graph depicting a comparison of ALT and AST activities between singly housed wildtype (WT) mice and Asc^{-/-} mice. FIG. 14D is a graph depicting a comparison of NAFLD activity plus histological scores for steatosis and inflammation between singly housed WT mice and Asc^{-/-} mice. FIG. 14E is a graph depicting a comparison of ALT and AST activities between singly housed wildtype (WT) mice and Nlrp3^{-/-} mice. FIG. 14F is a graph depicting a comparison of NAFLD activity plus histological scores for steatosis and inflammation between singly housed WT mice and Nlrp3^{-/-} mice. FIG. 14G is a graph depicting a comparison of ALT and AST activities between singly housed wildtype (WT) mice and IL18^{-/-} mice. FIG. 14H is a graph depicting a comparison of NAFLD activity plus histological scores for steatosis and inflammation between singly housed WT mice and IL18^{-/-} mice. Data represent two independent experiments (n=7-19 mice per treatment group). Error bars represent the s.e.m. of samples within a group. *p≦0.05, **p≦0.01, ***p≦0.001 (Student's t-test).

FIG. 15, comprising FIGS. 15A-15H, depicts how increased severity of NASH in Asc- and IL18-deficient mice is transmissible to co-housed wild-type animals. Asc^{-/-} or IL18^{-/-} mice and wild-type mice were co-housed for 4 weeks and then fed MCDD. FIG. 15A is a graph depicting ALT activity scores of singly housed wild-type mice (WT), wild-type mice cohoused with Asc^{-/-} mice (WT(Asc^{-/-})), and Asc^{-/-} mice co-housed with wild-type mice (Asc^{-/-} (WT)). FIG. 15B is a graph depicting AST activity scores of singly housed wild-type mice (WT), wild-type mice cohoused with Asc^{-/-} mice (WT(Asc^{-/-})), and Asc^{-/-} mice co-housed with wild-type mice (Asc^{-/-} (WT)). FIG. 15C is a graph depicting NAFLD activity histological scores of singly housed wild-type mice (WT), wild-type mice cohoused with Asc^{-/-} mice (WT(Asc^{-/-})), and Asc^{-/-} mice co-housed with wild-type mice (Asc^{-/-} (WT)). FIG. 15D is a series of images of representative haematoxylin and eosin-stained sections of livers of singly housed wild-type mice (WT), wild-type mice cohoused with Asc^{-/-} mice (WT(Asc^{-/-})), and Asc^{-/-} mice co-housed with wild-type mice (Asc^{-/-} (WT)). FIG. 15E is a graph depicting ALT activity scores of wild-type, WT(IL18^{-/-}) and IL18^{-/-} (WT) mice. FIG. 15F is a graph depicting AST activity scores of wild-type, WT(IL18^{-/-}) and IL18^{-/-} (WT) mice. FIG. 15G is a graph depicting NAFLD activity histological scores of wild-type, WT(IL18^{-/-}) and IL18^{-/-} (WT) mice. FIG. 15H is a series of images of representative haematoxylin and eosin-stained sections of livers of wild-type, WT(IL18^{-/-}) and IL18^{-/-} (WT). Data are representative of two independent experiments. Error bars represent s.e.m. Scale bars, 200 μm (FIGS. 15D and 15H). *p≦0.05, **p≦0.01, ***p≦0.001.

FIG. 16, comprising FIGS. 16A-16F, depicts how 16S rRNA sequencing demonstrates diet and co-housing associated changes in gut microbial ecology. FIG. 16A is a graph depicting a principal coordinates analysis (PCoA) of unweighted UniFrac distances of 16S rRNA sequences, demonstrating clustering according to co-housing status on principal coordinate 1 (PC1). FIG. 16B is a graph depicting PCoA of same plot as in FIG. 16A colored for experimental day. Mice were co-housed and fed a regular diet (R) for the first 32 days of the experiment (two time points taken at day 20 and 32) before being switched to MCDD (M, sampled at days 39, 46 and 51 of the experiment). FIGS. 16C-16F depict PCoA and bar graphs of family level taxa Prevotellaceae (FIG. 16C), Porphyromonadaceae (FIG. 16D), Bacteroidaceae, (FIG. 16E), and Erysipelotrichaceae (FIG. 16F) demonstrating diet- and microbiota-dependent differences in taxonomic representation. PCoA plots contain spheres representing a single faecal community coded according to relative representation of the taxon (blue represents relatively higher levels; red indicates lower levels). Bar graphs represent averaged taxonomic representation for singly or co-housed mouse while on either regular or MCD diet (n=8 for singly housed wild-type, n=12 co-housed Asc^{-/-} (WT) and WT(Asc^{-/-}) animals). *p≦0.05, **p≦0.01, p≦0.001 by t-test after Bonferroni correction for multiple hypotheses (n.d.=not detected; Reg. diet=regular diet).

FIG. 17, comprising FIGS. 17A-17H, depicts how the increased severity of NASH in Asc-deficient and co-housed wildtype animals is mediated by TLR4, TLR9 and TNF-α. Asc^{-/-} mice were cohoused with wild-type, Tnf^{-/-}, Tlr4^{-/-}, Tlr9^{-/-} or Tlr5^{-/-} mice for 4 weeks and then fed MCDD. FIG. 17A is a graph depicting ALT activity levels of Tlr4^{-/-} and Tlr4^{-/-} (Asc^{-/-}) mice. FIG. 17B is a graph depicting ALT activity levels of Tlr9^{-/-} and Thr9^{-/-} (Asc^{-/-}) mice. FIG. 17C is a graph depicting ALT activity levels of Tlr5^{-/-} and Tlr5^{-/-} (Asc^{-/-}) mice. FIG. 17D is a graph quantifying TLR4 agonists in portal vein sera from MCDD-fed wild-type, WT(Asc^{-/-}) and Asc^{-/-} animals. FIG. 17E is a series of transmission electron microscopy images of colon from wild-type and Asc^{-/-} mice. FIG. 17F is a graph depicting ALT activity scores of Tnf^{-/-}, WT(Asc^{-/-}) and Tnf^{-/-} (Asc^{-/-}) mice. FIG. 17G is a graph depicting NAFLD activity histological scores of Tnf^{-/-}, WT(Asc^{-/-}) and Tnf^{-/-} (Asc^{-/-}) mice. FIG. 17H is a graph depicting NAFLD activity histological scores of Tnf^{-/-}, WT(Asc^{-/-}) and Tnf^{-/-} (Asc^{-/-}) mice. Data are representative of two independent experiments. Error bars represent s.e.m. *p≦0.05, **p≦0.01, ***p≦0.001.

FIG. 18, comprising FIGS. 18A-18J, depicts how increased severity of NASH in Asc-deficient mice is transmissible to db/db by co-housing and is mediated by CCL5-induced intestinal inflammation, FIG. 18A is a graph depicting ALT activity scores of WT(Asc^{-/-}) and Ccl5^{-/-} (Asc^{-/-}) mice. FIG. 18B is a graph depicting AST activity scores of WT(Asc^{-/-}) and Ccl5^{-/-} (Asc^{-/-}) mice. FIG. 18C is a graph depicting NAFLD activity histological scores of WT(Asc^{-/-}) and Ccl5^{-/-} (Asc^{-/-}) mice. Data represents two independent experiments. FIG. 18D is a series of images of representative haematoxylin and eosin-stained sections of colon from db/db(WT) and db/db(Asc^{-/-}) mice. FIG. 18E is a series of images of representative haematoxylin and eosin-stained sections of terminal ileum from db/db(WT) and db/db(Asc^{-/-}) mice. FIG. 18F is a series of images of representative haematoxylin and eosin-stained sections of liver from db/db(WT) and db/db(Asc^{-/-}) mice. In FIGS. 18D-18F, db/db mice were co-housed with wild-type or Asc^{-/-} mice for 12 weeks and fed a standard chow diet. Mucosal and crypt hyperplasia (arrow). Hepatocyte degeneration (arrowhead). Scale bars, 500 μm (FIGS. 18D-18E), 200 μm (FIG. 18F). FIG. 18G is a graph depicting ALT activity scores of db/db(WT) and db/db(Asc^{-/-}) mice. FIG. 18H is a graph depicting AST activity scores of db/db(WT) and db/db(Asc^{-/-}) mice. FIG. 18I is a graph depicting NAFLD activity scores of db/db(WT) and db/db(Asc^{-/-}) mice. FIG. 18J is a graph depicting hepatic Tnf, IL6 and IL1b mRNA levels. Error bars represent s.e.m. *p≦0.05, **p≦0.01, ***p≦0.001.

FIG. 19, comprising FIGS. 19A-19J, depicts how Asc-deficient mice develop increased obesity and loss of glycaemic control on HFD. FIG. 19A is a graph depicting the weight of db/db(WT) or db/db(Asc^{-/-}) mice at 3 weeks of age and at 12 weeks of co-housing. FIG. 19B is a graph depicting body weights of Asc^{-/-} and wild-type mice co-housed for 4 weeks and then fed HFD. FIG. 19C is a graph depicting NAFLD histological activity score of Asc^{-/-} and wild-type mice co-housed for 4 weeks and then fed HFD. FIG. 19D is a graph depicting fasting plasma glucose levels after 11 weeks of HFD. FIG. 19E is a graph depicting insulin levels after 11 weeks of HFD. FIG. 19F is a graph depicting results of an intraperitoneal (i.p.) glucose tolerance test after 12 weeks of HFD. FIG. 19G is a graph depicting body weights in untreated mice and mice treated orally with antibiotics (Abx) for 3 weeks before HFD feeding for 12 weeks. FIG. 19H is a graph depicting fasting plasma levels after 8 weeks on a HFD in untreated mice and mice treated orally with antibiotics (Abx) for 3 weeks before HFD feeding for 12 weeks. FIG. 19I is a graph depicting insulin levels after 8 weeks on a HFD in untreated mice and mice treated orally with antibiotics (Abx) for 3 weeks before HFD feeding for 12 weeks. FIG. 19J is a graph depicting results of an intraperitoneal glucose tolerance test after 10 weeks of HFD. Error bars represent s.e.m. *p≦0.05, **p≦0.01, ***p≦0.001.

FIG. 20, comprising FIGS. 20A-20C, depicts the increased severity of NASH in inflammasome-deficient mice, but not in IL1r-deficient animals. To induce NASH, mice were fed with MCDD for 24 d. Their serum ALT and AST activities measured and NAFLD histological activity scores were determined. FIG. 20A is a series of graphs comparing ALT and AST activity between singly-housed wild-type (wt) mice and IL1r^{-/-} animals. FIG. 20B is a graph depicting a comparison NAFLD activity, plus histological scores for steatosis and inflammation, between singly-housed wild-type (wt) mice and IL1r^{-/-} animals. FIG. 20C is a series of images of representative hematoxylin and eosin (H&E)-stained sections of livers from wt, caspase-1^{-/-}, Asc^{-/-}, Nlrp3^{-/-}, IL18^{-/-}, and IL1r^{-/-} mice. Inflammatory foci are highlighted with an arrowhead. Data represent two independent experiments (n=7-19 mice/treatment group). Error bars represent the SEM of samples within a group. Scale bars=200 μm (K). *p≦0.05, **p≦0.01, ***p≦0.001 (Student's t test).

FIG. 21, comprising FIGS. 21A-21D, depicts changes in liver cellularity in MCDD-fed Asc-deficient mice and cohoused WT animals. Singly-housed WT, co-housed Asc^{-/-} (WT), and co-housed WT(Asc^{-/-}) animals were fed MCDD for 24 days to induce NASH, and hematopoietic cell subsets in liver were quantified by FACS. FIG. 21A is a graph depicting the total numbers of CD45⁺ cells, B cells (B220⁺), T cells (TCRβ⁺), CD4⁺ T cells, CD8⁺ T cells, NK cells (NK1.1⁺ TCRβ⁻), NKT cells (NK1.1⁺ TCRβ⁺), dendritic cells (CD11c⁺ CD11b⁻), mononuclear macrophages (MHCII⁺ CD11b⁺), and neutrophils (Gr1⁺). *p≦0.05, **p≦0.01, ***p≦0.001. between the WT single housed group and the co-housed WT(Asc^{-/-}) animals (Student's t test). Data is representative of two independent experiments. FIG. 21B is a graph depicting a comparison of serum ALT in WT and compound homozygous knockout Asc^{-/-};Rag^{-/-} mice. FIG. 21C is a graph depicting a comparison of serum AST in WT and compound homozygous knockout Asc^{-/-};Rag^{-/-} mice. FIG. 21B is a graph depicting a comparison of NAFLD activity histological scores for steatosis and inflammation in wt and compound homozygous knockout Asc^{-/-};Rag^{-/-} mice. Error bars represent the SEM of samples within a group. *p≦0.05, **p≦0.01, ***p≦0.001. (Student's t test).

FIG. 22, comprising FIGS. 22A-22L, depicts how activation of the NLRP3 inflammasome in hematopoietic cells and hepatocytes does not influence NASH severity. To induce NASH, mice were given MCDD for 24 days, and their serum ALT and AST activities, and NAFLD histological activity scores were determined. FIG. 22A is a graph depicting a comparison of ALT activity between chimeric mice generated with WT (WT>WT) and Nlrp3^{-/-} (Nlrp3^{-/-}>WT) bone marrow (BM). FIG. 22B is a graph depicting a comparison of AST activity between chimeric mice generated with WT (WT>WT) and Nlrp3^{-/-} (Nlrp3^{-/-}>WT) bone marrow (BM). FIG. 22C is a graph depicting a comparison of NAFLD activity histological scores for steatosis and inflammation between chimeric mice generated with WT (WT>WT) and Nlrp3^{-/-} (Nlrp3^{-/-}>WT) bone marrow (BM). FIG. 22D is a graph depicting a comparison of ALT activity between chimeric mice generated with WT (WT>WT) and Asc^{-/-} (Asc^{-/-}>WT) BM. FIG. 22E is a graph depicting a comparison of AST activity between chimeric mice generated with WT (WT>WT) and Asc^{-/-} (Asc^{-/-}>WT) BM. FIG. 22F is a graph depicting a comparison of NAFLD activity histological scores for steatosis and inflammation between chimeric mice generated with WT (WT>WT) and Asc^{-/-} (Asc^{-/-}>WT) BM. FIG. 22G is a graph depicting a comparison of serum ALT activities between WT;CD11c+-Cre and Nlrp3KI;CD11c-Cre mice. FIG. 22H is a graph depicting a comparison of serum AST activities between WT;CD11c+-Cre and Nlrp3KI;CD11c-Cre mice. FIG. 22I is a graph depicting a comparison of NAFLD activity histological scores for steatosis and inflammation activities between WT;CD11c+-Cre and Nlrp3KI;CD11c-Cre mice. FIG. 22J is a graph depicting a comparison of serum ALT activities between WT;albumin-Cre and Nrp3KI;albumin-Cre mice. FIG. 22K is a graph depicting a comparison of serum AST activities between WT; albumin-Cre and Nlrp3KI;albumin-Cre mice. FIG. 22L is a graph depicting a comparison of NAFLD activity histological scores for steatosis and inflammation activities between WT;albumin-Cre and Nlrp3KI;albumin-Cre mice. Error bars represent the s.e.m. of samples within a group. *p≦0.05, **p≦0.01, ***p≦0.001. (Student's t test)

FIG. 23, comprising FIGS. 23A-23L, depicts how the increased severity of NASH in caspase-1-, Nlrp3-, and Nlrp6-deficient mice is transmissible to co-housed wild-type animals. The study involved singly-housed WT mice and WT mice co-housed with caspase-1^{-/-})) animals. Animals were given MCDD for 24 days to induce NASH. FIG. 23A is a graph depicting a comparison of serum ALT activity between WT and (WT(caspase-1^{-/-})) animals. FIG. 23B is a graph depicting a comparison of serum AST activity between WT and (WT(caspase-1^{-/-})) animals. FIG. 23C is a graph depicting a comparison of serum ALT activity between WT and Nlrp3^{-/-} animals (WT(Nlrp3^{-/-})). FIG. 23D is a graph depicting a comparison of serum AST activity between WT and Nlrp3^{-/-} animals (WT(Nlrp3^{-/-})). FIG. 23E is a graph depicting a comparison of serum ALT activity between WT and Nlrp6^{-/-} animals (wt(Nlrp6^{-/-})). FIG. 23F is a graph depicting a comparison of serum AST activity between WT and Nlrp6^{-/-} animals (wt(Nlrp6^{-/-})). FIG. 23G is a graph depicting a comparison of serum ALT activity between WT and Nlrp4c^{-/-} mice (wt(Nlrp4c^{-/-}). FIG. 23H is a graph depicting a comparison of serum AST activity between WT and Nrp4c^{-/-} mice (wt(Nlrp4c^{-/-})). FIG. 23I is a graph depicting a comparison of serum ALT activity between WT and Nlrc4^{-/-} mice (wt(Nlrc4^{-/-})). FIG. 23J is a graph depicting a comparison of serum AST activity between WT and Nlrc4^{-/-} mice (wt(Nlrc4^{-/-})). FIG. 23K is a graph depicting a comparison of serum ALT activity between WT and Nlrp12^{-/-} mice (wt(Nlrp12^{-/-})). FIG. 23L is a graph depicting a comparison of serum AST activity between WT and Nlrp12^{-/-} mice (wt(Nlrp12^{-/-})). Error bars represent the s.e.m. of samples within a group (n=3-8 mice/group). *p≦0.05, **p≦0.01, ***p≦0.001. (Student's t test).

FIG. 24, comprising FIGS. 24A-24D, depicts how the increased severity of NASH in Asc and IL18-deficient mice and co-housed wild-type animals is abolished with antibiotic treatment. FIG. 24A is a graph depicting a comparison of serum ALT of WT(Asc^{-/-}) and Asc^{-/-}(WT) mice. FIG. 24B is a graph depicting a comparison of serum AST of WT(Asc^{-/-}) and Asc^{-/-}(WT) mice. FIG. 24C is a graph depicting a comparison of NAFLD activity histological scores for steatosis of WT(Asc^{-/-}) and Asc^{-/-}(WT) mice. FIG. 24D is a graph depicting a comparison of NAFLD activity histological scores for inflammation of WT(Asc^{-/-}) and Asc^{-/-}(WT) mice. Mice were untreated or treated orally with a combination of metronidazole and ciprofloxacin for 4 weeks. Inflammatory foci are highlighted with an arrowhead. Data are representative of two independent experiments (n=5-7 mice/treatment group). Error bars represent the s.e.m. of samples within a group. *p≦0.05, **p≦0.01, ***p≦0.001 (ANOVA).

FIG. 25, comprising FIGS. 25A-25H, depicts how the increased severity of NASH in Asc-deficient mice and co-housed wild-type animals is mediated by TLR4, TLR9. Asc⁻⁻ mice were co-housed with WT, Myd88^{-/-};Trif^{-/-}, Tlr4^{-/-}, Tlr9^{-/-}, or Tlr5^{-/-} mice for 4 weeks, after which time mice were fed MCDD for 24 days to induce NASH. FIG. 25A is a graph depicting a comparison of serum ALT activities from MCDD-fed WT(Asc^{-/-}) and Myd88^{-/-};Trif^{-/-}(Asc^{-/-}) mice. FIG. 25B is a graph depicting a comparison of serum AST activities from MCDD-fed WT(Asc^{-/-}) and Myd88^{-/-};Trif^{-/-}(Asc^{-/-}) mice. Data in FIGS. 25A-25B are representative of two independent experiments. FIG. 25C is a graph depicting a comparison of serum AST levels from MCDD-fed Tlr4^{-/-}(Asc^{-/-}) animals and their singly-housed counterparts. FIG. 25D is a graph depicting a comparison of NAFLD activity histological scores for steatosis and inflammation from MCDD-fed Tlr4^{-/-}(Asc^{-/-}) animals and their singly-housed counterparts. FIG. 25E is a graph depicting a comparison of serum AST levels from MCDD-fed Tlr9^{-/-}(Asc^{-/-}) animals and their singly-housed counterparts, FIG. 25F is a graph depicting a comparison of NAFLD activity histological scores for steatosis and inflammation from MCDD-fed Tlr9^{-/-} (Asc^{-/-}) animals and their singly-housed counterparts. FIG. 25G is a graph depicting a comparison of serum AST levels from MCDD-fed Tlr5^{-/-}(Asc^{-/-}) animals and their singly-housed counterparts. FIG. 25H is a graph depicting a comparison of NAFLD activity histological scores for steatosis and inflammation from MCDD-fed Tlr5^{-/-} (Asc^{-/-}) animals and their singly-housed counterparts. Data represent two independent experiments. Error bars represent the SEM of samples within a group. *p≦0.05, **p≦0.01, ***p≦0.001 (Student's t test).

FIG. 26, comprising FIGS. 26A-26C, depicts how the increased severity of NASH in Asc-deficient mice and co-housed wild-type animals is mediated by TLR agonist influx into portal circulation. Asc^{-/-} mice were co-housed with WT mice for 4 weeks, after which time mice were fed MCDD for 24 days to induce NASH. FIG. 26A is a graph depicting a comparison of TLR2 agonists. FIG. 26B is a graph depicting a comparison of TLR9 agonists. Portal vein sera were obtained at the time of sacrifice of singly-housed MCDD-fed WT min ice, co-housed WT(Asc^{-/-}) animals and singly-housed Asc^{-/-} animals. Data represent two independent experiments. FIG. 26C is a series of representative transmission electron microscopy images taken from colonic sections prepared from WT (top, ×8200) and Asc^{-/-} animals (bottom, ×6500). Error bars represent the SEM of samples within a group. *p≦0.05, **p≦0.01, ***p≦0.001 (ANOVA).

FIG. 27, comprising FIGS. 27A-27G, depicts that Tnfα expression is increased in Asc^{-/-}, IL18^{-/-}, but not in IL1r^{-/-} mice, during NASH. FIG. 27A is a graph depicting a comparison of hepatic Tnfα, IL6, and IL1β mRNA levels in singly-housed WT and Asc^{-/-} mice. FIG. 27B is a graph depicting a comparison of hepatic Tnfα, IL6, and IL1β mRNA levels in singly-housed WT and IL18^{-/-} mice. FIG. 27C is a graph depicting a comparison of hepatic Tnfα, IL6, and IL1β mRNA levels in singly-housed WT and ILr^{-/-} mice. FIG. 27D is a graph depicting a comparison of hepatic Tnfα, IL6, and IL1β mRNA levels in singly-housed WT mice versus WT mice that were previously co-housed with Asc^{-/-} animals (wt(Asc^{-/-})). FIG. 27E is a graph depicting a comparison of hepatic Tnfα, IL6, and IL1β mRNA levels in singly-housed WT mice versus WT mice that were previously co-housed with IL18^{-/-} animals (wt(IL18^{-/-})). Mice were housed for four weeks prior to NASH induction. FIG. 27F is a graph depicting AST serum levels from singly-housed Tnfα^{-/-} mice, and co-housed WT mice (wt(Asc^{-/-}) or Tnfα^{-/-} mice co-housed with Asc^{-/-} animals (Tnfα^{-/-} (Asc^{-/-}). FIG. 27G is a series of images of representative H&E-stained sections of livers from singly-housed Tnfα^{-/-} mice, and co-housed WT mice (wt(Asc^{-/-}) or Tnfα^{-/-} mice co-housed with Asc^{-/-} animals (Tnfα^{-/-}(Asc^{-/-}). Scale bars=200 μm. Data are representative for two independent experiments. Error bars represent the SEM of samples within a group. *p≦0.05, **p≦0.01, ***p≦0.001 (Student's t test).

FIG. 28, comprising FIGS. 28A-28F, depicts that intestinal inflammation associated with an Asc^{-/-} gut microbiota increases the influx of TLR agonists into the portal circulation. To induce NASH, mice were given MCDD for 24 d, and their serum ALT and AST activities, and NAFLD histological activity scores, were determined. FIG. 28A is a graph depicting a comparison of ALT activities between separately housed WT and Ccl5^{-/-} mice. FIG. 28B is a graph depicting a comparison of AST activities between separately housed WT and Ccl5^{-/-} mice. FIG. 28A is a graph depicting a comparison of NAFLD activity histological scores for steatosis and inflammation between separately housed WT and Ccl5^{-/-} mice. (n=8 animals surveyed/group). In FIGS. 28D-28F, WT or Ccl5^{-/-} mice were co-housed with Asc^{-/-} mice for 4 weeks after which time mice were fed MCDD for 24 d to induce NASH. FIG. 28D is a graph depicting a comparison of TLR4 agonists in portal vein sera. FIG. 28E is a graph depicting a comparison of TLR9 agonists in portal vein sera. FIG. 28F is a graph depicting a comparison of TLR2 agonists in portal vein sera. Portal vein sera was collected from MCDD-treated, co-housed WT(Asc^{-/-}) and Ccl5^{-/-}(Asc^{-/-}) mice. Error bars represent the s.e.m. of samples within a group (n=6 animals surveyed/group). *p≦0.05, **p≦0.01, ***p≦0.001 (Student's t test).

FIG. 29, comprising FIGS. 29A-29D, depicts that Nlrc4^{-/-}-deficient mice have normal weight gain rate and glycemic control on HFD. Age-matched male Nlrc4^{-/-} mice and wt mice were fed a 60% HFD. FIG. 29A is a graph depicting the body weights of mice for the indicated time. Body weights were monitored weekly. FIG. 29B is a graph depicting results of glucose tolerance tests performed in wt mice and Nlrc4^{-/-} mice after 10 weeks of HFD. FIG. 29C is a graph depicting the measured levels of fasting (14 h) blood glucose measured after 8 weeks on the HFD. FIG. 29D is a graph depicting measured insulin levels measured after 8 weeks on the HFD. (n=8-10 mice/group). Error bars represent the SEM of samples within a group.

FIG. 30 is a series of images depicting that Asc-deficient mice co-housed wt mice develop increased steatosis on HFD. Representative images are of hematoxylin and eosin (H&E)-stained sections of livers from WT, WT(Asc^{-/-}), and Asc^{-/-} mice. Scale bars=200 μm.

FIG. 31, comprising FIGS. 31A-31E, depicts how antibiotic treatment leads to reduction in taxa associated with HFD. FIG. 31A is a graph depicting Asc^{-/-} and WT mice, which were or were not treated with ciprofloxacin and metronidazole for 4 weeks before being switched to a high fat diet. Time points were taken after being fed HFD for 1 and 8 weeks. FIG. 31B is a series of graphs depicting PCoA and showing reduction in Prevotellaceae after antibiotic treatment. FIG. 31C is a series of graphs depicting PCoA and showing reduction in Porphyromonadaceae after antibiotic treatment. FIG. 31D is a series of graphs depicting PCoA and showing reduction in Bacteroidaceae after antibiotic treatment. FIG. 31E is a series of graphs depicting how Enterococcaceae were noted to increase in representation after antibiotic treatment.

FIG. 32 is a table depicting the average bacterial taxonomic representation of Asc^{-/-}(WT) and WT(Asc^{-/-}) or singly housed mice fed a regular or MCDD (see FIG. 16). Values are expressed as averages per group with standard deviation in parentheses. P values, as determined by t test and corrected for multiple hypothesis testing by Bonferroni correction, are shown comparing groups.

FIG. 33 is a table depicting the average bacterial taxonomic representation as determined by 16S sequencing of mice that either were or were not treated with 4 weeks of antibiotics and fed a high fat diet for 1 or 8 weeks. Values are expressed as averages per group with standard deviation in parenthesis. P values of comparisons between groups were determined by t-test with correction for multiple hypotheses.

FIG. 34 is a table depicting the average bacterial taxonomic representation as determined by 16S sequencing of Nlrc4^{-/-} and WT mice. Values are expressed as averages per group with standard deviation in parentheses. P values of comparisons between groups were determined by t-test with correction for multiple hypotheses.

FIG. 35 is a table depicting bacterial taxa whose representation significantly correlates with the enhanced colitogenic fecal microbiota of inflammasome-deficient mice. The header for each column in the Table provides a description of housing conditions and genotypes of various groups of mice that are described in the main text and the indicated main text Figure. Note that the single-caged WT mice listed in each column are specifically those WT mice used as controls for the set of experiments involving the indicated knockout animals that were cohoused with WT. Genotypes and housing conditions that resulted in an enhanced colitogenic microbiota are indicated by a ‘Yes’ followed by the total number of mice within the groups represented in the column that exhibited this phenotype. None of members of any of the groups in any of the columns shown in the Table were exposed to DSS prior to fecal sampling and 16S rRNA-based analysis. The representation of various phylogenetic groups of bacteria in the fecal microbiota of mice belonging to the groups indicated within a column header are noted. Representation is expressed the mean percentage of total OTUs assigned to the indicated taxon. If the number of animals in which that taxon is present is less than the total number of mice indicated after a ‘No’ or Yes' in the column header then that number is shown in parenthesis within the cell. The significance of the difference in representation of a taxon between different groups of mice was determined using ANOVA and G-test after FDR correction for multiple hypotheses. (n.s.=Not significant, n.d.=not determined).

### DETAILED DESCRIPTION

The present invention relates to the discovery that the disruption of inflammasome function leads to an altered microbiota population that affects the development and progression of an inflammatory disease and disorder. Thus, the invention relates to compositions and methods for detecting and determining the relative proportions of the constituents of a subject's microbiota, to determine whether a subject's microbiota is an altered microbiota associated with an inflammatory disease or disorder. In various embodiments, the relative proportions of the constituents of a subject's microbiota are indicative of an altered microbiota population associated with an inflammatory disease or disorder. In some embodiments, the detection of an altered microbiota population in the subject is used to diagnose the subject as having, or as at risk of developing, an inflammatory disease or disorder. In various embodiments, the inflammatory diseases and disorders associated with an altered microbiota population include, but are not limited to, at least one of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), or non-alcoholic steatohepatitis (NASH).

Further, the present invention relates to methods of modifying an altered microbiota population in a subject in need thereof. In some embodiments, the invention provides compositions and methods for supplementing constituents of an altered microbiota that are under-represented in the altered microbiota, as compared with a normal microbiota, to restore the subject's microbiota to a normal microbiota. In other embodiments, the invention provides compositions and methods for diminishing constituents of an altered microbiota that are over-represented in the altered microbiota, as compared with a normal microbiota, to restore the subject's microbiota to a normal microbiota. In further embodiments, the invention provides compositions and methods for both supplementing constituents of an altered microbiota that are under-represented in the altered microbiota, as well as diminishing constituents of an altered microbiota that are over-represented in the altered microbiota, as compared with a normal microbiota, to restore the subject's microbiota to a normal microbiota.

Further, the present invention relates to the discovery that the level and activity of CCL5 is increased in a subject having an altered microbiota associated with an inflammatory disease or disorder. Thus, in one embodiment, the invention provides compositions and methods for treating a subject in need thereof, by modulating CCL5 to restore the level of CCL5 in the subject to a normal level. In other embodiments, the invention provides compositions and methods for treating a subject in need thereof, by modulating at least one of the receptors of CCL5 (e.g., CCR1, CCR3, CCR4, CCR5 or GPR75), to restore CCL5 activity in the subject to a normal level. In fulther embodiments, the invention provides compositions and methods for treating a subject in need thereof, by modulating both CCL5 and at least one of its receptors (e.g., CCR1, CCR3, CCR4, CCR5 or GPR75), to restore CCL5 activity in the subject to a normal level. Interfering with the interaction between CCL5 and at least one of its receptors (i.e., CCL5, CCR1, CCR3, CCR4, CCR5AND GPR75), thereby diminishes inflammation. In various embodiments, the inflammatory diseases and disorders that are treatable by the compositions and methods of the invention described herein include, but are not limited to, at least one of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH).

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of pathology, for the purpose of diminishing or eliminating those signs or symptoms.

As used herein, “treating a disease or disorder” means reducing the severity and/or frequency with which a sign or symptom of the disease or disorder is experienced by a patient. Disease and disorder are used interchangeably herein.

The phrase “biological sample” as used herein, is intended to include any sample comprising a cell, a tissue, or a bodily fluid in which expression of a nucleic acid or polypeptide is present or can be detected. Samples that are liquid in nature are referred to herein as “bodily fluids.” Biological samples may be obtained firom a patient by a variety of techniques including, for example, by scraping or swabbing an area of the subject or by using a needle to obtain bodily fluids. Methods for collecting various body samples are well known in the art.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of VH (variable heavy chain immunoglobulin) genes from an animal.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “DNA” as used herein is defined as deoxyribonucleic acid.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “microbiota” is used to refer to the community of microbes that occupy the digestive tract of a subject.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “probiotic” refers to a population of beneficial bacteria that can be administered to a subject to aid in the restoration of a subject's microbiota.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to the discovery that the disruption of inflammasome function leads to an altered microbiota population that affects the development and progression of an inflammatory disease and disorder. Thus, the invention relates to compositions and methods for detecting and determining the relative proportions of the constituents of a subject's microbiota, to determine whether a subject's microbiota is an altered microbiota associated with an inflammatory disease or disorder. Further, the present invention relates to methods of modifying an altered microbiota population in a subject in need thereof. Still further, the present invention relates to the discovery that the level and activity of CCL5 is increased in a subject having an altered microbiota associated with an inflammatory disease or disorder. Thus, in one embodiment, the invention provides compositions and methods for treating a subject in need thereof, by modulating CCL5 to restore the level of CCL5 in the subject to a normal level.

### Diagnostics

In one embodiment, the invention is a method for determining the relative proportions of the constituents of a subject's microbiota, to determine whether a subject's microbiota is an altered microbiota associated with an inflammatory disease or disorder. In various embodiments, the relative proportions of the constituents of a subject's microbiota are indicative of an altered microbiota population associated with an inflammatory disease or disorder. In some embodiments, the detection of an altered microbiota population in the subject is used to diagnose the subject as having, or as at risk of developing, an inflammatory disease or disorder. In various embodiments, the inflammatory disease or disorder associated with an altered microbiota population include, but are not limited to, at least one of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH).

Specific alterations in a subject's microbiota can be detected using various methods, including without limitation quantitative PCR or high-throughput sequencing methods which detect relative proportion of bacterial genetic markers in a total heterogeneous bacterial population. In particular embodiments, the bacterial genetic marker is at least some portion of the 16S rRNA. In various embodiments, the relative proportion of particular constituent bacterial phyla, classes, orders, families, genera, and species present in the microbiota of a subject is determined. In some embodiments, the relative proportion of particular constituent bacterial phyla, classes, orders, families, genera, and species present in the microbiota of a subject is determined and compared with that of a normal microbiota. In various embodiments, the comparator normal microbiota is, by way of examples, a microbiota of a subject known to be free of an inflammatory disorder, an historical norm, or an average microbiota of the population of which the subject is a member.

In some embodiments, an increase in at least one of Prevotellaceae, TM7, Porphyromonadaceae, and Erysipelotrichaceae, as compared with a normal microbiota, is indicative of an altered microbiota associated with an inflammatory disease or disorder. In other embodiments, a decrease in at least one of Lactobacillus spp., as compared with a normal microbiota, is indicative of an altered microbiota associated with an inflammatory disease or disorder. In a further embodiment, an increase in at least one of Prevotellaceae, TM7, Porphyromonadaceae, and Erysipelotrichaceae, as compared with a normal microbiota, and a decrease in at least one of Lactobacillus spp., as compared with a normal microbiota, is indicative of an altered microbiota associated with an inflammatory disease or disorder.

In one embodiment, the method of the invention is a diagnostic assay for diagnosing an inflammatory disease or disorder associated with an altered microbiota in a subject in need thereof, by determining whether an altered microbiota is present in a biological sample obtained from the subject. The results of the diagnostic assay can be used alone, or in combination with other information from the subject, or from the biological sample obtained from the subject.

In the assay methods of the invention, a test biological sample from a subject is assessed for the presence of an altered microbiota associated with an inflammatory disease or disorder. The test biological sample can be an in vitro sample or an in vivo sample. In various embodiments, the subject is a human subject, and may be of any race, sex and age. Representative subjects include those who are suspected of having an altered microbiota associated with an inflammatory disease or disorder, those who have been diagnosed with an altered microbiota associated with an inflammatory disease or disorder, those whose have an altered microbiota associated with an inflammatory disease or disorder, those who have had an altered microbiota associated with an inflammatory disease or disorder, those who at risk of a recurrence of an altered microbiota associated with an inflammatory disease or disorder, those who at risk of a flare of an altered microbiota associated with an inflammatory disease or disorder, and those who are at risk of developing an altered microbiota associated with an inflammatory disease or disorder.

In one embodiment, the test sample is a sample containing at least a fragment of a bacterial nucleic acid. The term, “fragment,” as used herein, indicates that the portion of a nucleic acid (e.g., DNA, RNA) that is sufficient to identify it as comprising a bacterial nucleic acid.

In some embodiments, the test sample is prepared from a biological sample obtained from the subject. The biological sample can be a sample from any source which contains a bacterial nucleic acid (e.g., DNA, RNA), such as a bodily fluid or fecal sample or a combination thereof. A biological sample can be obtained by any suitable method. In some embodiments, a biological sample containing bacterial DNA is used. In other embodiments, a biological sample containing bacterial RNA is used. The biological sample can be used as the test sample; alternatively, the biological sample can be processed to enhance access to nucleic acids, or copies of nucleic acids, and the processed biological sample can then be used as the test sample. For example, in various embodiments, nucleic acid is prepared from a biological sample, for use in the methods. Alternatively or in addition, if desired, an amplification method can be used to amplify nucleic acids comprising all or a fragment of an RNA or DNA in a biological sample, for use as the test sample in the assessment of the presence, absence and proportion of particular types of bacteria present in the sample.

In some embodiments, hybridization methods, such as Southern analysis, Northern analysis, or in situ hybridizations, can be used (see Current Protocols in Molecular Biology, Ausubel, F. et al., eds., John Wiley & Sons, including all supplements). For example, the presence of nucleic acid from a particular type of bacteria can be determined by hybridization of nucleic acid to a nucleic acid probe. A “nucleic acid probe,” as used herein, can be a DNA probe or an RNA probe.

The nucleic acid probe can be, for example, a fuil-length nucleic acid molecule, or a portion thereof, such as an oligonucleotide of at least 15, 30, 50, 100, 250 or 500 nucleotides in length and sufficient to specifically hybridize under stringent conditions to appropriate target RNA or DNA. The hybridization sample is maintained under conditions which are sufficient to allow specific hybridization of the nucleic acid probe to RNA or DNA. Specific hybridization can be performed under high stringency conditions or moderate stringency conditions, as appropriate. In a preferred embodiment, the hybridization conditions for specific hybridization are high stringency. More than one nucleic acid probe can also be used concurrently in this method. Specific hybridization of any one of the nucleic acid probes is indicative of the presence of the particular type of bacteria of interest, as described herein.

In Northern analysis (see Current Protocols in Molecular Biology, Ausubel, F. et al., eds., John Wiley & Sons, supra), the hybridization methods described above are used to identify the presence of a sequence of interest in an RNA, such as unprocessed, partially processed or fully processed rRNA. For Northern analysis, a test sample comprising RNA is prepared from a biological sample from the subject by appropriate means. Specific hybridization of a nucleic acid probe, as described above, to RNA from the biological sample is indicative of the presence of the particular type of bacteria of interest, as described herein.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a particular bacterial nucleic acid sequence. Hybridization of the PNA probe to a nucleic acid sequence is indicative of the presence of the particular type of bacteria of interest.

Direct sequence analysis can also be used to detect a bacterial nucleic acid of interest. A sample comprising DNA or RNA can be used, and PCR or other appropriate methods can be used to amplify all or a fragment of the nucleic acid, and/or its flanking sequences, if desired. The bacterial nucleic acid, or a friagment thereof, is determined, using standard methods.

In another embodiment, arrays of oligonucleotide probes that are complementary to target bacterial nucleic acid sequences can be used to detect and identify bacterial nucleic acids. For example, in one embodiment, an oligonucleotide array can be used. Oligonucleotide arrays typically comprise a plurality of different oligonucleotide probes that are coupled to a surface of a substrate in different knuown locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and scanned for particular bacterial nucleic acids.

Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein. In brief, a target bacterial nucleic acid sequence is amplified by well-known amplification techniques, e.g., PCR. Typically, this involves the use of primer sequences that are complementary to the target sequence. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Other methods of nucleic acid analysis can be used to detect bacterial nucleic acids of interest. Representative methods include direct manual sequencing (1988, Church and Gilbert, Proc. Natl. Acad. Sci. USA 81:1991-1995; 1977, Sanger et al., Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (1981, Sheffield et al., Proc. Natl. Acad. Sci. USA 86:232-236), mobility shift analysis (1989, Orita et al., Proc. Natl. Acad. Sci. USA 86:2766-2770; 1987, Rosenbaum and Reissner, Biophys. Chem. 265:1275; 1991, Keen et al., Trends Genet. 7:5); restriction enzyme analysis (1978, Flavell et al., Cell 15:25; 1981, Geever, et al., Proc. Natl. Acad. Sci. USA 78:5081); heteroduplex analysis; chemical mismatch cleavage (CMC) (1985, Cotton et al., Proc. Natl. Acad. Sci. USA 85:4397-4401); RNase protection assays (1985, Myers, et al., Science 230:1242); use of polypeptides which recognize nucleotide mismatches, such as E. coli mutS protein (see, for example, U.S. Pat. No. 5,459,039); Luminex xMAP™ technology; high-throughput sequencing (HTS) (2011, Gundry and Vijg, Mutat Res, doi: 10.1016/j.mrfinmm.2011.10.001); next-generation sequencing (NGS) (2009, Voelkerding et al., Clinical Chemistry 55:641-658; 2011, Su et al., Expert Rev Mol. Diagn. 11:333-343; 2011, Ji and Myllykangas, Biotechnol Genet Eng Rev 27:135-158); ion semiconductor sequencing (2011, Rusk, Nature Methods doi:10.1038/nmeth.f.330; 2011, Rothberg et al., Nature 475:348-352) and/or allele-specific PCR, for example. These and other methods can be used to identify the presence of one or more bacterial nucleic acids of interest, in a biological sample obtained from a subject. In one embodiment of the invention, the methods of assessing a biological sample for the presence or absence of a particular nucleic acid sequence, as described herein, are used to diagnose an altered microbiota associated with an inflammatory disease or disorder in a subject in need thereof.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the biological sample using known techniques. Nucleic acid herein refers to RNA, including mRNA, and DNA, including genomic DNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a fresh or fixed biological sample.

Routine methods also can be used to extract DNA from a biological sample, including, for example, phenol extraction. Alternatively, genomic DNA can be extracted with kits such as the QIAamp™, Tissue Kit (Qiagen, Chatsworth, Calif.), the Wizard™ Genomic DNA purification kit (Promega, Madison, Wis.), the Puregene DNA Isolation System (Gentra Systems, Inc., Minneapolis, Minn.), and the A.S.A.P.™ Genomic DNA isolation kit (Boehringer Mannheim, Indianapolis, Ind.).

There are many methods known in the art for the detection of specific nucleic acid sequences and new methods are continually reported. A great majority of the known specific nucleic acid detection methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids of exogenous organisms in a body sample known in the art are the hybridization methods as exemplified by U.S. Pat. No. 6,159,693 and No. 6,270,974, and related patents. To briefly summarize one of those methods, a nucleic acid probe of at least 10 nucleotides, preferably at least 15 nucleotides, more preferably at least 25 nucleotides, having a sequence complementary to a desired region of the target nucleic acid of interest is hybridized in a sample, subjected to depolymerizing conditions, and the sample is treated with an ATP/luciferase system, which will luminesce if the nucleic sequence is present. In quantitative Southern blotting, levels of the target nucleic acid can be determined.

A further process for the detection of hybridized nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed before. Most preferably the detection of the duplex is done using at least one primer directed to the target nucleic acid. In yet another embodiment of PCR, the detection of the hybridized duplex comprises electrophoretic gel separation followed by dye-based visualization.

DNA amplification procedures by PCR are well known and are described in U.S. Pat. No. 4,683,202. Briefly, the primers anneal to the target nucleic acid at sites distinct from one another and in an opposite orientation. A primer annealed to the target sequence is extended by the enzymatic action of a heat stable DNA polymerase. The extension product is then denatured friom the target sequence by heating, and the process is repeated. Successive cycling of this procedure on both DNA strands provides exponential amplification of the region flanked by the primers.

Amplification is then performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

The expression “specifically hybridizing in stringent conditions” refers to a hybridizing step in the process of the invention where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods.

Hybridization is typically accomplished by annealing the oligonucleotide probe or primer to the DNA under conditions of stringency that prevent non-specific binding but permit binding of this DNA which has a significant level of homology with the probe or primer.

Among the conditions of stringency is the melting temperature (Tm) for the amplification step using the set of primers, which is in the range of about 55° C. to about 70° C. Preferably, the Tm for the amplification step is in the range of about 59° C. to about 72° C. Most preferably, the Tm for the amplification step is about 60° C.

Typical hybridization and washing stringency conditions depend in part on the size (i.e., number of nucleotides in length) of the DNA or the oligonucleotide probe, the base composition and monovalent and divalent cation concentrations (Ausubel et al., 1997, eds Current Protocols in Molecular Biology).

In a preferred embodiment, the process for determining the quantitative and qualitative profile according to the present invention is characterized in that the amplifications are real-time amplifications performed using a labeled probe, preferably a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tin, which is in the range of about 65° C. to 75° C. Preferably, the Tm for any one of the hydrolysis-probes of the present invention is in the range of about 67° C. to about 70° C. Most preferably, the Tm applied for any one of the hydrolysis-probes of the present invention is about 67° C.

In another preferred embodiment, the process for determining the quantitative and qualitative profile according to the present invention is characterized in that the amplification products can be elongated, wherein the elongation products are separated relative to their length. The signal obtained for the elongation products is measured, and the quantitative and qualitative profile of the labeling intensity relative to the elongation product length is established.

The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

In one aspect, the invention includes a primer that is complementary to a target bacterial nucleic acid, and more particularly the primer includes 12 or more contiguous nucleotides substantially complementary to the sequence flanking the nucleic acid sequence of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target flanking nucleotide sequence In another aspect, the length of the primer can vary in length, preferably about 15 to 28 nucleotides in length (e.g., 16, 17, 18, 19, 20, 21, 22, 23, 24, 25, 26, or 27 nucleotides in length).

The present invention also pertains to kits useful in the methods of the invention. Such kits comprise components useful in any of the methods described herein, including for example, hybridization probes or primers (e.g., labeled probes or primers), reagents for detection of labeled molecules, means for amplification of nucleic acids, means for analyzing a nucleic acid sequence, and instructional materials. For example, in one embodiment, the kit comprises components useful for analysis of a bacterial nucleic acid of interest present in a biological sample obtained from a subject. In a preferred embodiment of the invention, the kit comprises components for detecting one or more of the bacterial nucleic acids of interest present in a biological sample obtained from a subject.

### Therapeutic Methods

In conjunction with the diagnostic methods, the present invention also provides therapeutic methods for treating an inflammatory disease or disorder associated with an altered microbiota by modifying the microbiota to that observed in a healthy subject. In some embodiments, the methods supplement the numbers of the types of bacteria that are under-represented in the altered microbiota. In other embodiments, the methods diminish the numbers of the types of bacteria that are overrepresented in the altered microbiota. In a further embodiment, the methods both supplement the numbers of the types of bacteria that are under-represented in the altered microbiota, and diminish the numbers of the types of bacteria that are overrepresented in the altered microbiota. In various embodiments, the inflammatory diseases and disorders treatable by the methods of the invention include, but are not limited to: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), or non-alcoholic steatohepatitis (NASH).

In some embodiments, modification of the altered microbiota is achieved by administering to a subject in need thereof a therapeutically effective amount of a probiotic composition comprising an effective amount of at least one type of bacteria, or a combinations of several types of bacteria, wherein the administered bacteria supplements the number of the types of bacteria which are under-represented in the altered microbiota, as compared with a normal microbiota.

Bacteria administered according to the methods of the present invention can comprise live bacteria. One or several different types of bacteria can be administered concurrently or sequentially. Such bacteria can be obtained from any source, including being isolated from a microbiota and grown in culture using known techniques. Non-limiting examples of types of bacteria that can be administered to supplement bacteria that are under-represented in the altered microbiota include, for example, Lactobacillus spp.

In certain embodiments, the administered bacteria used in the methods of the invention further comprise a buffering agent. Examples of useful buffering agents include sodium bicarbonate, milk, yogurt, infant formula, and other dairy products.

Administration of a bacterium can be accomplished by any method suitable for introducing the organisms into the desired location. The bacteria can be mixed with a carrier and (for easier delivery to the digestive tract) applied to a liquid or to food. The carrier material should be non-toxic to the bacteria as wells as the subject. Preferably, the carrier contains an ingredient that promotes viability of the bacteria during storage. The formulation can include added ingredients to improve palatability, improve shelf-life, impart nutritional benefits, and the like.

The dosage of the administered bacteria will vary widely, depending upon the nature of the inflammatory disease or disorder, the character of subject's altered microbiota, the subject's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve colonization of the gastrointestinal tract with the desired bacteria. In some embodiments, the dose ranges from 10⁶-10¹⁰ CFU. In other embodiments, the dose ranges from 10⁴, and 10⁵ CFU.

In certain embodiments, the present invention relates to a method for modifying an altered microbiota comprising administering to a subject in need of such treatment, an effective amount of at least one gastric, esophageal, or intestinal bacterium, or combinations thereof. In a preferred embodiment, the bacteria are administered orally. Alternatively, bacteria can be administered rectally or by enema.

One of the organisms contemplated for administration to modify the altered microbiota is at least one Lactobacillus spp. In certain embodiments, the bacteria administered in the therapeutic methods of the invention comprise administration of a combination of organisms.

While it is possible to administer a bacteria for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Although there are no physical limitations to delivery of the formulations of the present invention, oral delivery is preferred for delivery to the digestive tract because of its ease and convenience, and because oral formulations readily accommodate additional mixtures, such as milk, yogurt, and infant formula. For delivery to colon, bacteria can be also administered rectally or by enema.

In other embodiments, modification of the altered microbiota is achieved by administering to a subject in need thereof a therapeutically effective amount of antibiotic composition comprising an effective amount of at least one antibiotic, or a combinations of several types of antibiotics, wherein the administered antibiotic diminishes the number of at least one type of bacteria that is over-represented in the altered microbiota, as compared with a normal microbiota. In various embodiments, the at least one type of bacteria that is diminished using the methods of the invention includes at least one of Prevotellaceae, TM7, Porphyromonadaceae, and Erysipelotrichaceae,

The type and dosage of the administered antibiotic will vary widely, depending upon the nature of the inflammatory disease or disorder, the character of subject's altered microbiota, the subject's medical history, the frequency of administration, the manner of administration, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In various embodiments, the administered antibiotic is at least one of lipopeptide, fluoroquinolone, ketolide, cephalosporin, amikacin, gentamicin, kanamycin, neomycin, netilmicin, paromomycin, streptomycin, tobramycin, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, cefltezole, cefaclor, cefamandole, cefmetazole, cefonicid, cefotetan, cefoxitin, cefprozil, cefuroxime, cefuzonam, cefeapene, cefdaloxime, cefdinir, cefditoren, cefetamet, cefixime, cefinenoxime, cefodizime, cefotaxime, cefpimizole, cefpodoxime, cefteranm, ceftibuten, ceftiofur, ceftiolene, ceftizoxime, cefiriaxone, cefoperazone, ceftazidime, cefclidine, cefepime cefluprenam, cefoselis, cefozopran, cefpirome, cefquinome, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftaroline, ceftioxide, cefuracetime, imipenem, primaxin, doripenem, meropenem, ertapenem, flumequine, nalidixic acid, oxolinic acid, piromidic acid pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, spartfloxacin, temafloxacin, tosufloxacin, clinafloxacin, gemifloxacin, sitafloxacin, trovafloxacin, prulifloxacin, azithromycin, erythromycin, clarithromycin, dirithromycin, roxithromycin, telithromycin, amoxicillin, ampicillin, bacampicillin, carbenicillin, cloxacillin, dicloxacillin, flucloxacillin, mezlocillin, nafcillin, oxacillin, penicillin g, penicillin v, piperacillin, pivampicillin, pivmecillinam, ticarcillin, sulfamethizole, sulfamethoxazole, sulfisoxazole, trimethoprim-sulfamethoxazole, demeclocycline, doxycycline, minocycline, oxytetracycline, tetracycline, linezolid, clindamycin, metronidazole, vancomycin, vancocin, mycobutin, rifampin, nitrofurantoin, chloramphenicol, or derivatives thereof.

In a further embodiment, modification of the altered microbiota is achieved by both administering at least one type of bacteria to supplement the numbers of at least one type of bacteria that is under-represented in the altered microbiota, and administering at least one antibiotic to diminish the numbers of at least one type of bacteria that is over-represented in the altered microbiota.

### Therapeutic Modulator Compositions and Methods of Use

In various embodiments, the present invention includes modulator compositions and methods of preventing and treating an inflammatory disease or disorder associated with an altered microbiota. In various embodiments, the modulator compositions and methods of treatment of the invention modulate the level or activity of a gene, or gene product, associated with an inflammatory disease or disorder associated with an altered microbiota. In some embodiments, the modulator composition of the invention is an activator that increases the level or activity of a gene, or gene product, associated with an inflammatory disease or disorder associated with an altered microbiota. In other embodiments, the modulator composition of the invention is an inhibitor that decreases the level or activity of a gene, or gene product, associated with an inflammatory disease or disorder associated with an altered microbiota.

It will be understood by one skilled in the art, based upon the disclosure provided herein, that modulating a gene, or gene product, encompasses modulating the level or activity of a gene, or gene product, associated with an inflammatory disease or disorder associated with an altered microbiota, including, but not limited to, transcription, translation, splicing, enzymatic activity, binding activity, or combinations thereof. Thus, modulating the level or activity of a gene, or gene product, associated with an inflammatory disease or disorder associated with an altered microbiota includes, but is not limited to, modulating transcription, translation, splicing, or combinations thereof, of a nucleic acid; and it also includes modulating any activity of polypeptide gene product as well.

In various embodiments, the modulated gene, or gene product, that is associated with an inflammatory disease or disorder associated with an altered microbiota, is at least one of: CCL5, NLRP6, NLRP3 and IL-18^{-/-}.

Modulation of a gene, or gene product, can be assessed using a wide variety of methods, including those disclosed herein, as well as methods known in the art or to be developed in the future. That is, the routineer would appreciate, based upon the disclosure provided herein, that modulating the level or activity of a gene, or gene product, can be readily assessed using methods that assess the level of a nucleic acid encoding a gene product (e.g., mRNA), the level of polypeptide gene product present in a biological sample, the activity of polypeptide gene product present in a biological sample, or combinations thereof.

The modulator compositions and methods of the invention that modulate the level or activity of a gene, or gene product, associated with an inflammatory disease or disorder associated with an altered microbiota, include, but should not be construed as being limited to, a chemical compound, a protein, a peptide, a peptidomemetic, an antibody, a ribozyme, a small molecule chemical compound, an antisense nucleic acid molecule (e.g., siRNA, miRNA, etc.), or combinations thereof. One of skill in the art would readily appreciate, based on the disclosure provided herein, that a modulator composition encompasses a chemical compound that modulates the level or activity of a gene, or gene product, associated with intracranial aneurysm. Additionally, a modulator composition encompasses a chemically modified compound, and derivatives, as is well known to one of skill in the chemical arts.

The modulator compositions and methods of the invention include antibodies. The antibodies of the invention include a variety of forms of antibodies including, for example, polyclonal antibodies, monoclonal antibodies, intracellular antibodies (“intrabodies”), Fv, Fab and F(ab)2, single chain antibodies (scFv), heavy chain antibodies (such as camelid antibodies), synthetic antibodies, chimeric antibodies, and humanized antibodies. In one embodiment, the antibody of the invention is an antibody that specifically binds to a polypeptide gene product of a gene associated with an inflammatory disease or disorder associated with an altered microbiota. In another embodiment, the antibody of the invention is an antibody that specifically binds to molecule that interacts with a polypeptide gene product of a gene associated with an inflammatory disease or disorder associated with an altered microbiota.

Further, one of skill in the art would, when equipped with this disclosure and the methods exemplified herein, appreciate that modulators include such modulators as discovered in the future, as can be identified by well-known criteria in the art of pharmacology, such as the physiological results of modulation of the genes, and gene products, as described in detail herein and/or as known in the art. Therefore, the present invention is not limited in any way to any particular modulator composition as exemplified or disclosed herein; rather, the invention encompasses those modulator compositions that would be understood by the routineer to be useful as are known in the art and as are discovered in the future.

Further methods of identifying and producing modulator compositions are well known to those of ordinary skill in the art, including, but not limited, obtaining a modulator from a naturally occurring source (i.e., Streptomyces sp., Pseudomonas sp., Stylotella aurantium). Alternatively, a modulator can be synthesized chemically. Further, the routineer would appreciate, based upon the teachings provided herein, that a modulator composition can be obtained from a recombinant organism. Compositions and methods for chemically synthesizing modulators and for obtaining them from natural sources are well known in the art and are described in the art.

One of skill in the art will appreciate that a modulator can be administered as a small molecule chemical, a polypeptide, a peptide, an antibody, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is modulator of a gene, or gene product, associated with an inflammatory disease or disorder associated with an altered microbiota. (Sambrook et al., 2001, Molecular Cloning: A Laboratory Manual, Cold Spring Harbor Laboratory, New York; Ausubel et al., 1997, Current Protocols in Molecular Biology, John Wiley & Sons, New York).

Antisense oligonucleotides are DNA or RNA molecules that are complementary to some portion of an RNA molecule. When present in a cell, antisense oligonucleotides hybridize to an existing RNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of an antisense oligonucleotide to modulate the amount of a gene, or gene product, associated with an inflammatory disease or disorder associated with an altered microbiota, thereby modulating the amount or activity of the gene product.

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expression can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that the modulators of the invention can be administered singly or in any combination. Further, the modulators of the invention can be administered singly or in any combination in a temporal sense, in that they may be administered concurrently, or before, and/or after each other. One of ordinary skill in the art will appreciate, based on the disclosure provided herein, that the modulator compositions of the invention can be used to prevent or to treat intracranial aneurysm, and that a modulator composition can be used alone or in any combination with another modulator to effect a therapeutic result. In various embodiments, any of the modulators of the invention described herein can be administered alone or in combination with other modulators of other molecules associated with an inflammatory disease or disorder associated with an altered microbiota. Non-limiting examples of modulators that can be used in combination with the modulators and methods of the invention include: steroids, glucocorticoid steroids, corticosteroids, non-steroidal anti-inflammatory drugs, and antibodies that specifically bind to pro-inflammatory mediators and/or their receptors, including α-IL-1, α-TNFα, α-IFNγ, α-TNFβ, α-IL4, α-IL5, α-IL6, α-IL10, and α-IL13.

It will be appreciated by one of skill in the art, when armed with the present disclosure including the methods detailed herein, that the invention is not limited to treatment of an inflammatory disease or disorder associated with an altered microbiota, that is already established. Particularly, the disease or disorder need not have manifested to the point of detriment to the subject; indeed, the disease or disorder need not be detected in a subject before treatment is administered. That is, significant signs or symptoms of the disease or disorder do not have to occur before the present invention may provide benefit. Therefore, the present invention includes a method for preventing an inflammatory disease or disorder associated with an altered microbiota, in that a modulator composition, as discussed previously elsewhere herein, can be administered to a subject prior to the onset of the disease or disorder, thereby preventing the disease or disorder. The preventive methods described herein also include the treatment of a subject that is in remission for the prevention of a recurrence an inflammatory disease or disorder associated with an altered microbiota.

One of skill in the art, when armed with the disclosure herein, would appreciate that the prevention of an inflammatory disease or disorder associated with an altered microbiota, encompasses administering to a subject a modulator composition as a preventative measure against the development of, or progression of, an inflammatory disease or disorder associated with an altered microbiota. As more fully discussed elsewhere herein, methods of modulating the level or activity of a gene, or gene product, associated with an inflammatory disease or disorder associated with an altered microbiota, encompass a wide plethora of techniques for modulating not only the level and activity of polypeptide gene products, but also for modulating expression of a nucleic acid, including either transcription, translation, or both.

Additionally, as disclosed elsewhere herein, one skilled in the art would understand, once armed with the teaching provided herein, that the present invention encompasses methods of treating, or preventing, a wide variety of diseases, disorders and pathologies where modulating the level or activity of a gene, or gene product, that is associated with an inflammatory disease or disorder associated with an altered microbiota, mediates, treats or prevents the disease or disorder. Various methods for assessing whether a disease relates to a gene, or gene product, that is associated with an inflammatory disease or disorder associated with an altered microbiota are described elsewhere herein and are known in the art. Further, the invention encompasses treatment or prevention of such diseases discovered in the future.

The invention encompasses administration of a modulator of a gene, or gene product, that is associated with an inflammatory disease or disorder associated with an altered microbiota, to practice the methods of the invention; the skilled artisan would understand, based on the disclosure provided herein, how to formulate and administer the appropriate modulator composition to a subject. Indeed, the successful administration of the modulator has been reduced to practice as exemplified herein. However, the present invention is not limited to any particular method of administration or treatment regimen.

### Inhibition of CCL5

In various embodiments, the present invention includes compositions and methods for treating an inflammatory disease and disorder associated with an altered microbiota by diminishing the expression level, or activity level, of CCL5. In other embodiments, the invention includes compounds and methods for treating for treating an inflammatory disease and disorder associated with an altered microbiota by interfering with the interaction between CCL5 and at least one of its receptors (e.g., CCR1, CCR3, CCR4, CCR5 and GPR75).

It will be understood by one skilled in the art, based upon the disclosure provided herein, that a decrease in the level of CCL5 encompasses the decrease of CCL5 expression. Additionally, the skilled artisan would appreciate, once armed with the teachings of the present invention, that a decrease in the level of CCL5 includes a decrease in CCL5 activity. Thus, decreasing the level or activity of CCL5 includes, but is not limited to, decreasing transcription, translation, or both, of a nucleic acid encoding CCL5; and it also includes decreasing any activity of CCL5 as well.

Inhibition of CCL5 can be assessed using a wide variety of methods, including those disclosed herein, as well as methods well-known in the art or to be developed in the future. That is, the routineer would appreciate, based upon the disclosure provided herein, that decreasing the level or activity of CCL5 can be readily assessed using methods that assess the level of a nucleic acid encoding CCL5 (e.g., mRNA) and/or the level of CCL5 protein present in a biological sample.

One skilled in the art, based upon the disclosure provided herein, would understand that the invention is useful in treating an inflammatory disease and disorder associated with an altered microbiota in subjects who have an altered microbiota, whether or not the subject also being treated with other medication. Further, the skilled artisan would further appreciate, based upon the teachings provided herein, that the inflammatory diseases and disorders associated with an altered microbiota treatable by the compositions and methods described herein encompass any pathology associated with an altered microbiota where CCL5, CCR1, CCR3, CCR4, CCR5 or GPR75 plays a role.

A CCL5 inhibitor can include, but should not be construed as being limited to, a chemical compound, a protein, a peptide, a peptidomemetic, an antibody, a ribozyme, a small molecule chemical compound, and an antisense nucleic acid molecule (e.g., siRNA, miRNA, etc.). One of skill in the art would readily appreciate, based on the disclosure provided herein, that a CCL5 inhibitor encompasses a chemical compound that decreases the level or activity of CCL5. Additionally, a CCL5 inhibitor encompasses a chemically modified compound, and derivatives, as is well known to one of skill in the chemical arts.

Further, one of skill in the art would, when equipped with this disclosure and the methods exemplified herein, appreciate that a CCL5 inhibitor includes such inhibitors as discovered in the future, as can be identified by well-known criteria in the art of pharmacology, such as the physiological results of inhibition of CCL5 as described in detail herein and/or as known in the art. Therefore, the present invention is not limited in any way to any particular CCL5 inhibitor as exemplified or disclosed herein; rather, the invention encompasses those inhibitors that would be understood by the routineer to be useful as are known in the art and as are discovered in the future.

Further methods of identifying and producing CCL5 inhibitors are well known to those of ordinary skill in the art, including, but not limited, obtaining an inhibitor from a naturally occurring source (i.e., Streptomyces sp., Pseudomonas sp., Stylotella aurantium). Alternatively, a CCL5 inhibitor can be synthesized chemically. Further, the routineer would appreciate, based upon the teachings provided herein, that a CCL5 inhibitor can be obtained from a recombinant organism. Compositions and methods for chemically synthesizing CCL5 inhibitors and for obtaining them from natural sources are well known in the art and are described in the art.

One of skill in the art will appreciate that an inhibitor can be administered as a small molecule chemical, a protein, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an inhibitor of CCL5. (Sambrook et al., 2001, Molecular Cloning: A Laboratory Manual, Cold Spring Harbor Laboratory, New York; Ausubel et al., 1997, Current Protocols in Molecular Biology, John Wiley & Sons, New York).

One of skill in the art will realize that diminishing the amount or activity of a molecule that itself increases the amount or activity of CCL5 can serve in the compositions and methods of the present invention to decrease the amount or activity of CCL5.

Antisense oligonucleotides are DNA or RNA molecules that are complementary to some portion of an mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of an antisense oligonucleotide to diminish the amount of CCL5, or to diminish the amount of a molecule that causes an increase in the amount or activity of CCL5, thereby decreasing the amount or activity of CCL5.

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing CCL5, or of a gene expressing a protein that increases the level or activity of CCL5, can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that inhibitors of CCL5 can be administered singly or in any combination. Further, CCL5 inhibitors can be administered singly or in any combination in a temporal sense, in that they may be administered simultaneously, before, and/or after each other. One of ordinary skill in the art will appreciate, based on the disclosure provided herein, that CCL5 inhibitors can be used to treat for treating an inflammatory disease or disorder associated with an altered microbiota, and that an inhibitor can be used alone or in any combination with another inhibitor to effect a therapeutic result.

It will be appreciated by one of skill in the art, when armed with the present disclosure including the methods detailed herein, that the invention is not limited to treatment of an inflammatory disease or disorder associated with an altered microbiota that are already established. Particularly, the disease or disorder need not have manifested to the point of detriment to the subject; indeed, the pathology need not be detected in a subject before treatment is administered. That is, significant inflammation associated with an altered microbiota does not have to occur before the present invention may provide benefit. Therefore, the present invention includes a method for preventing an inflammatory disease and disorder associated with an altered microbiota in a subject, in that a CCL5 inhibitor, as discussed previously elsewhere herein, can be administered to a subject prior to the onset of the disease or disorder, thereby preventing or diminishing the severity of the disease or disorder. The preventive methods described herein also include the treatment of a subject that is in remission for the prevention of a recurrence.

One of skill in the art, when armed with the disclosure herein, would appreciate that the prevention of inflammatory diseases and disorders associated with an altered microbiota encompasses administering to a subject a CCL5 inhibitor as a preventative measure against an inflammatory diseases and disorders associated with an altered microbiota. As more fully discussed elsewhere herein, methods of decreasing the level or activity of CCL5 encompass a wide plethora of techniques for decreasing not only CCL5 activity, but also for decreasing expression of a nucleic acid encoding CCL5.

Additionally, as disclosed elsewhere herein, one skilled in the art would understand, once armed with the teaching provided herein, that the present invention encompasses a method of preventing a wide variety of diseases, disorders and pathologies where a decrease in expression and/or activity of CCL5 mediates, treats or prevents the disease or disorder. Methods for assessing whether a disease relates to increased levels or activity of CCL5 are known in the art. Further, the invention encompasses treatment or prevention of such diseases discovered in the future.

The invention encompasses administration of an inhibitor of CCL5 to practice the methods of the invention; the skilled artisan would understand, based on the disclosure provided herein, how to formulate and administer the appropriate CCL5 inhibitor to a subject. Indeed, the successful administration of the CCL5 inhibitor has been reduced to practice as exemplified herein. However, the present invention is not limited to any particular method of administration or treatment regimen.

### Inhibition of a Receptor of CCL5

In various embodiments, the present invention includes compositions and methods of treating an inflammatory disease or disorder associated with an altered microbiota by diminishing the expression level, or activity level, of at least one of the receptors of CCL5 (e.g., CCR1, CCR3, CCR4, CCR5 and GPR75). In other embodiments, the invention includes compounds and methods for treating an inflammatory disease or disorder associated with an altered microbiota by interfering with the interaction between at least one of CCR1, CCR3, CCR4, CCR5 and GPR75, and their ligand, CCL5. In still further embodiments, the invention includes compounds and methods for treating an inflammatory disease or disorder associated with an altered microbiota by interfering with signal transduction through at least one of CCR1, CCR3, CCR4, CCR5 and GPR75.

It would be understood by one skilled in the art, based upon the disclosure provided herein, that a decrease in the level of at least one CCL5 receptor encompasses the decrease in expression at least one CCL5 receptor. Additionally, the skilled artisan would appreciate, once armed with the teachings of the present invention, that a decrease in the level of at least one CCL5 receptor includes a decrease in the activity of at least one CCL5 receptor. Thus, decreasing the level or activity of at least one CCL5 receptor includes, but is not limited to, decreasing transcription, translation, or both, of a nucleic acid encoding a CCL5 receptor; and it also includes decreasing any activity of a CCL5 receptor as well, including, but not limited to, ligand binding activity.

Inhibition of a CCL5 receptor can be assessed using a wide variety of methods, including those disclosed herein, as well as methods well-known in the art or to be developed in the future. That is, the routineer would appreciate, based upon the disclosure provided herein, that decreasing the level or activity of a CCL5 receptor can be readily assessed using methods that assess the level of a nucleic acid encoding a CCL5 receptor (e.g., mRNA) and/or the level of a CCL5 receptor protein present in a biological sample. Examples of known CCL5 receptor inhibitors useful in the compositions and methods of the invention included, but are not limited to, aplaviroc, vicriviroc and maraviroc.

One skilled in the art, based upon the disclosure provided herein, would understand that the invention is useful in treating inflammatory diseases and disorders associated with an altered microbiota in subjects who have an altered microbiota, whether or not the subject also being treated with other medication. Further, the skilled artisan would further appreciate, based upon the teachings provided herein, that the inflammatory diseases and disorders associated with an altered microbiota treatable by the compositions and methods described herein encompass any inflammatory disease and disorder associated with an altered microbiota where CCL5, CCR3, CCR4, CCR5 or GPR75 plays a role.

A CCL5 receptor inhibitor can include, but should not be construed as being limited to, a chemical compound, a protein, a peptide, a peptidomemetic, an antibody, a ribozyme, a small molecule chemical compound, and an antisense nucleic acid molecule (e.g., siRNA, miRNA, etc.). One of skill in the art would readily appreciate, based on the disclosure provided herein, that a CCL5 receptor inhibitor encompasses a chemical compound that decreases the level or activity of a CCL5 receptor. Additionally, a CCL5 receptor inhibitor encompasses a chemically modified compound, and derivatives, as is well known to one of skill in the chemical arts.

Further, one of skill in the art would, when equipped with this disclosure and the methods exemplified herein, appreciate that a CCL5 receptor inhibitor includes such inhibitors as discovered in the future, as can be identified by well-known criteria in the art of pharmacology, such as the physiological results of inhibition of a CCL5 receptor as described in detail herein and/or as known in the art. Therefore, the present invention is not limited in any way to any particular CCL5 receptor inhibitor as exemplified or disclosed herein; rather, the invention encompasses those inhibitors that would be understood by the routineer to be useful as are known in the art and as are discovered in the future.

Further methods of identifying and producing CCL5 receptor inhibitors are well known to those of ordinary skill in the art, including, but not limited, obtaining an inhibitor from a naturally occurring source (i.e., Streptomyces sp., Pseudomonas sp., Stylotella aurantium). Alternatively, a CCL5 receptor inhibitor can be synthesized chemically. Further, the routineer would appreciate, based upon the teachings provided herein, that a CCL5 receptor inhibitor can be obtained from a recombinant organism. Compositions and methods for chemically synthesizing a CCL5 receptor inhibitor and for obtaining them from natural sources are well known in the art and are described in the art.

One of skill in the art will appreciate that an inhibitor can be administered as a small molecule chemical, a protein, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an inhibitor of a CCL5 receptor. (Sambrook et al., 2001, Molecular Cloning: A Laboratory Manual, Cold Spring Harbor Laboratory, New York; Ausubel et al., 1997, Current Protocols in Molecular Biology, John Wiley & Sons, New York).

One of skill in the art will realize that diminishing the amount or activity of a molecule that itself increases the amount or activity of a CCL5 receptor can serve in the compositions and methods of the present invention to decrease the amount or activity of a CCL5 receptor.

Antisense oligonucleotides are DNA or RNA molecules that are complementary to some portion of an mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of an antisense oligonucleotide to diminish the amount of a CCL5 receptor, or to diminish the amount of a molecule that causes an increase in the amount or activity of a CCL5 receptor, thereby decreasing the amount or activity of a CCL5 receptor.

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a CCL5 receptor, or of a gene expressing a protein that increases the level or activity of a CCL5 receptor, can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that inhibitors of a CCL5 receptor can be administered singly or in any combination. Further, CCL5 receptor inhibitors can be administered singly or in any combination in a temporal sense, in that they may be administered simultaneously, before, and/or after each other. One of ordinary skill in the art will appreciate, based on the disclosure provided herein, that a CCL5 receptor inhibitor can be used to treat an inflammatory disease or disorder associated with an altered microbiota, and that an inhibitor can be used alone or in any combination with another inhibitor to effect a therapeutic result.

It will be appreciated by one of skill in the art, when armed with the present disclosure including the methods detailed herein, that the invention is not limited to treatment of an inflammatory disease or disorder that is associated with an altered microbiota that is already established. Particularly, the disease or disorder need not have manifested to the point of detriment to the subject; indeed, the disease or disorder need not be detected in a subject before treatment is administered. That is, significant inflammation associated with an altered microbiota does not have to occur before the present invention may provide benefit. Therefore, the present invention includes a method for preventing an inflammatory disease or disorder associated with an altered microbiota in a subject, in that a CCL5 receptor inhibitor, as discussed previously elsewhere herein, can be administered to a subject prior to the onset of the disease or disorder, thereby preventing, or diminishing the severity of, the disease or disorder.

One of skill in the art, when armed with the disclosure herein, would appreciate that the prevention of inflammatory diseases and disorders associated with an altered microbiota encompasses administering to a subject a CCL5 receptor inhibitor as a preventative measure against an inflammatory disease or disorder associated with an altered microbiota. As more fully discussed elsewhere herein, methods of decreasing the level or activity of a CCL5 receptor encompass a wide plethora of techniques for decreasing not only a CCL5 receptor activity, but also for decreasing expression of a nucleic acid encoding a CCL5 receptor.

Additionally, as disclosed elsewhere herein, one skilled in the art would understand, once armed with the teaching provided herein, that the present invention encompasses a method of preventing a wide variety of diseases, disorders and pathologies where a decrease in expression and/or activity of a CCL5 receptor mediates, treats or prevents the disease or disorder. Methods for assessing whether a disease relates to increased levels or activity of a CCL5 receptor are known in the art. Further, the invention encompasses treatment or prevention of such diseases discovered in the future.

The invention encompasses administration of an inhibitor of a CCL5 receptor to practice the methods of the invention; the skilled artisan would understand, based on the disclosure provided herein, how to formulate and administer the appropriate CCL5 receptor inhibitor to a subject. Indeed, the successful administration of the CCL5 receptor inhibitor has been reduced to practice as exemplified herein. However, the present invention is not limited to any particular method of administration or treatment regimen.

### Pharmaceutical Compositions

Modulator compositions useful for treatment and/or prevention of an inflammatory disease or disorder associated with an altered microbiota, can be formulated and administered to a subject for treatment of an inflammatory disease or disorder associated with an altered microbiota disclosed herein are now described.

The invention encompasses the preparation and use of pharmaceutical modulator compositions comprising a modulator compound useful for treatment of an inflammatory disease or disorder associated with an altered microbiota disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically-acceptable carrier” means a chemical composition with which an appropriate inhibitor thereof, may be combined and which, following the combination, can be used to administer the appropriate inhibitor thereof, to a subject.

The pharmaceutical compositions useful for practicing the invention may be administered to deliver a dose of between about 0.1 ng/kg/day and 100 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate inhibitor, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate inhibitor thereof, according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 plg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### NLRP6 Inflammasome Regulates Colonic Microbial Ecology and Risk for Colitis

Described herein is a regulatory sensing system in the colon that is dependent on the NLRP6 inflammasome. Genetic deletion of components of this sensing system is shown herein to have drastic consequences on the composition of the microbial communities, leading to a shift toward a proinflammatory configuration that drives spontaneous and induced colitis.

Although not wishing to be bound by any particular theory, on a molecular level it appears unlikely that the evolutionarily conserved, innate mucosal immune arm possesses the ability to distinctly identify the myriad bacterial, archaeal, and eukaryotic microbial phylotypes and virotypes that comprise the gut microbiota and differentiate autochthonous (entrenched) or allochthonous (transient/nomadic) components of this community that act as commensals or mutualists, from those that act as pathogens. Rather, this function may be achieved by sensing signals that are related to tissue integrity or factors released by tissue damage that serve as “danger signals” promoting activation of an innate response (Matzinger, 2007, Nat. Immunol. 8:11-13). Inflammasomes are capable of fulfilling this task, as they can be activated by many microbial ligands, but also by host-derived factors released upon cell or tissue damage, such as uric acid, ATP, and hyaluronan (Schroder et al., 2010, Proc. Natl. Acad. Sci, USA 98:13249-13254). NLRP6 assembly in the colonic epithelial compartment may be driven by a low level of these substances or by yet unidentified molecules signaling tissue integrity, resulting in local production of IL-18^{-/-}. Interestingly, in the rat, NLRP6, caspase-1, ASC^{-/-}, and pro-IL-18^{-/-} are absent at embryonic day 16 (E16) and first appear at E20, with the processed form of IL-18 emerging in the gut during the early postnatal period (Kempster et al., 2011, Am. J. Physiol. Gastrointest, Liver Physiol. 300:G253-G263), coinciding with the time of colonization of the gut ecosystem.

Although not wishing to be bound by any particular theory, dysbiosis may contribute to IBD by expansion of colitogenic strains such as entero-invasive E. coli (Darfeuille-Michaud et al., 2004, Gastroenterology 127:412-421), by reduction of tolerogenic strains such as Faecalibacterimn prausnitzi (Sokol et al., 2008, Proc. Natl. Acad. Sci. USA 105:16731-16736), or through a combination of both mechanisms. In the studies described herein, a colitogenic microbiota with altered representation of distinct bacterial members formed in the intestines of NLRP6-deficient mice; this microbiota was transferred across generations within a kinship and could displace the gut microbiota of cohoused immunocompetent mice. Once this community was horizontally transmitted to suckling or adult WT mice, it could persist. Compared to WT mice, NLRP6 inflammasome-deficient mice exhibited both quantitative and qualitative changes in numerous taxa, including increased representation of members of Prevotellaceae and TM7, and reductions in members of genus Lactobacillus in the Firmicutes phylum.

There are several intriguing links between the abundance of Prevotellaceae and TM7 and human diseases. Prevotellaceae has been implicated in periodontal disease (Kumar et al., 2003, J. Dent. Res. 82:338-344), and several reports have documented prominent representation of this group in samples from IBD patients (Kleessen et al., 2002, Scand. J. Gastroenterol. 37:1034-1041; Lucke et al., 2006, J. Med. Microbiol. 55:617-624). Prevetellaceae might disrupt the mucosal barrier function through production of sulfatases that actively degrade mucus oligosaccharides (Wright et al., 2000, FEMS Microbiol. Lett. 190:73-79); these enzymes are elevated in intestinal biopsies from IBD patients (Tsai et al., 1995, Gut 36:570-576). Though they have not been cultured, members of the TM7 phylum have been identified in 16S rRNA surveys of terrestrial and aquatic microbial communities as well in human periodontal disease (Brinig et al., 2003, Appl. Environ. Microbiol. 69:1687-1694; Marcy et al., 2007, Proc. Natl. Acad. Sci. USA 104:11889-11894; Ouverney et al., 2003, Appl. Environ. Microbiol. 69:6294-6298) and in IBD patients (Kuehbacher et al., 2008, J. Med. Mirobiol. 57:1569-1576). Defining the nature of the interactions of Prevotellaceae and TM7 with the NLRP6 inflammasome may provide insights about probiotic interventions that may mitigate microbiota-mediated enhanced inflammatory responses.

Varying degrees of tissue injury and subsequent inflammation may result in shifting the balance between protective and detrimental effects, depending on the experimental condition and the inflammatory context. However, inflammasome-driven effects on the colonic microbiota, as revealed in the studies described herein, add yet another layer of regulation that affects and effects initiation of autoinflammation. As such, exacerbation in colitis severity in single-housed inflammasome-deficient mice may, in fact, involve defects in tissue regeneration, but this histopathological process may be dramatically influenced by the effects imposed by altered elements in the microbiota, including, for example, the enhanced representation of Prevotellaceae in the crypt. Thus, although not wishing to be bound by any particular theory, the fundamental role of the microbiota in shaping processes related to tissue damage, regeneration, and stress response might offer an explanation for the opposing results between these studies. Furthermore, these results described herein suggest that prolonged cohousing (or littermate controls) should be used when NLRs and other innate receptors are studied: allowing for equilibration of differences in gut microbial ecology that may exist between groups of mice and allow investigators to determine which features of their phenotypes can be ascribed to the microbiota. Indeed, using cohousing conditions, it was demonstrated that the NLRC4 inflammasome is a direct negative regulator of colonic epithelial cell tumorigenesis that is not driven by the microbiota (Hu et al., 2010, Proc. Natl. Acad. Sci, USA 107:21635-21640).

These results show that the resultant aberrant microbiota promotes local epithelial induction of CCL5 transcription as a downstream mechanism, ultimately leading to an exaggerated autoinflammatory response. CCL5 is potently induced by bacterial and viral infections and, in turn, induces massive recruitment of a variety of innate and adaptive immune cells carrying CCR1, CCR3, CCR4, and CCR5 (Mantovani et al., 2004, Trends Immunol. 25:677-686). Interestingly, both NOD2 and TLRs have been shown to induce CCL5 transcription (Bérubé et al., 2009, Cell. Signal. 21:448-456; Werts et al., 2007, Eur. J. Immunol. 37:2499-2508).

Recent studies have highlighted the importance of the gut microbiota in the pathogenesis of various autoimmune disorders that manifest outside of the gastrointestinal tract. The studies described herein indicate that deficiencies in the NLRP6 pathway should be added to the list of host genetic factors that may drive disease-specific alterations in the microbiota, which in turn may promote disease in these hosts or in individuals who have been exposed to these microbial communities and who have also experienced disruption in their gut epithelial barrier function due to a variety of insults.

The materials and methods employed in these experiments are now described.

### Mice

NLRP6^{-/-} mice were generated by replacing exons 1 and 2 with a neomycin selection cassette (IRESnlslacZ/MC1neo). For cohousing experiments, age- and gender-matched WT and knockout mice were cohoused at 1:1 ratios for 4 weeks. ASC^{-/-} (Pycardtm1Flv (Sutterwala et al., 2006, Immunity 24:317-327), Casp1^{-/-} mice (Casp1^{tm1Flv} (Kuida et al., 1995, Science 267:2000-2003), NLRC4^{-/-} (Nlrc4^{tm1Gln}, (Lara-Tejero et al., 2006, J. Exp. Med. 203:1407-1412), AIM2^{-/-} (Aim2^{Gt(CSG445)Byg}, (Rathinam et al., 2010, Nat. Ihnmunol. 11:395-402), NLRP12^{-/-} (Nlrpl2tm1Jpyt, (Arthur et al., 2010, J. Immunol. 185:4515-4519), IL-18^{-/-} (IL18^{tm1Aki}, (Takeda et al., 1998, Immunity 8:383-390), IL-1R^{-/-} (ILr^{1tm1Imx}, (Glaccum et al., 1997, J. Immunol. 159:3364-3371), and IL-1b^{-/-} mice (IL1b^{tm1Lvp}, (Zheng et al., 1995, Immunity 3:9-19) were described in previous publications.

The generation of NLRP1−/− mice has not been published (S.C.E., unpublished data). NLRP6^{-/-}, ASC^{-/-}, Casp1^{-/-}, and IL-18^{-/-} mice were backcrossed at least 10 times to C56Bl/6. IL-1R^{-/-} mice were backcrossed five times to C56Bl/6, while IL-1b^{-/-} mice were on a 129S7 background (and hence used for cohousing purposes only). WT C56Bl/6 mice were purchased from NCl. Where indicated, WT mice were also used that had been bred in our mouse barrier facility. All mice were specific pathogen-free, maintained under a strict 12 hour light cycle (lights on at 7:00 am and off at 7:00 pm), and given a regular chow diet (Harlan, diet #2018) ad libitum.

For cohousing experiments, age- and gender-matched WT and knockout mice were co-housed in new cages at 1:1 ratios for 4 weeks. For cross-fostering experiments, newborn mice were exchanged between ASC−/− and WT mothers within 24 hours of birth. Mice were weaned between postnatal days 21-28. For bone marrow chimera experiments, mice were given a sublethal dose of total body irradiation (2×5.5 Gy, 3 hours apart). 16 hours later mice were transplanted with 4×10⁶ unseparated bone marrow cells. Mice were analyzed 7-8 weeks later.

For antibiotic treatment, mice were given either (i) a combination of vancomycin (1 g/l), ampicilin (1 g/l), kanamycin (1 g/l), and metronidazole (1 g/l) or (ii) a combination of ciprofloxacin (0.2 g/l) and metronidazole (1 g/l) for 3 weeks in their drinking water. All antibiotics were obtained from Sigma Aldrich (St. Louis, Mo.). All experimental procedures were approved by the local IACUC.

### DSS Colitis

Mice were treated with 2% (w/v) DSS (M.W.=36,000-50,000 Da; MP Biomedicals) in their drinking water for 7 days followed by regular access to water.

### Colonoscopy

Colonoscopy was performed using a high resolution mouse video endoscopic system (‘Coloview’, Carl Storz, Tuttlingen, Germany). The severity of colitis was blindly scored using MEICS (Murine Endoscopic Index of Colitis Severity) which is based on five parameters: granularity of mucosal surface; vascular pattern; translucency of the colon mucosa; visible fibrin; and stool consistency (Becker et al., 2006, Nat. Protoc. 1:2900-2904).

### Histology

Colons were fixed in Bouin's medium and embedded in paraffin. Blocks were serially sectioned along the cephalocaudal axis of the gut to the level of the lumen; the next 5 mm-thick section was stained with hematoxylin and eosin. Each section was scored by a pathologist who was blinded with respect to the origin of the sample: scoring was based on the degree of inflammation (location and extent), edema, mucosal ulceration, hyperplasia, crypt loss or abscess (Hu et al., 2010, Proc. Natl. Acad. Sci. USA 107:21635-21640; O'Connor et al., 2009, Nat. Immunol. 10:603-609). Severity scores ranged from 0 to 5 with 0 being normal and 5 being most severe. Individual scores were assigned for each parameter, and then averaged for a final score per sample. Digital light microscopic images were recorded with a Zeiss Axio Imager.A 1 microscope (Thornwood, N.Y.), AxioCam MRc5 camera and AxioVision 4.7.1 imaging software (Carl Zeiss Microimaging). Results are displayed as percent involvement of colon (inflamed colon area) and by score of the most severe lesion in each sample (pathological severity score).

### Immunofluorescence Staining

Frozen sections of colons from WT and NLRP6−/− mice were blocked in 10% fetal bovine serum for 1 hour at room temperature. Slides were incubated at 4° C. for 16 hours with primary antibody to NLRP6 (clone E20, goat IgG, Santa Cruz Biotechnologies, Santa Cruz, Calif.) at 2 mg/ml, followed by incubation with 1:800 Alexa Fluor 647-labeled rabbit anti-goat secondary antibody (Invitrogen, Grand Island, N.Y., Molecular Probes, Eugene Oreg.) for 2 hours at 4° C. Sections were counterstained with 4,6-diamidino-2-phenylindole (DAPI) for nuclear staining. Slides were dried and mounted, using ProLong Antifade mounting medium (Invitrogen, Grand Island, N.Y., Molecular Probes, Eugene Oreg.). Slides were visualized using a Leica TCS SP5 confocal microscope.

### Immunoprecipitation and Western Blot Analysis

Colons were excised and washed thoroughly by flushing several times with PBS, opened longitudinally, transferred into HBSS+2 mM EDTA, and shaken for 20 min at 37° C. Subsequently, colons were washed 3 times with PBS and these washes were pooled with the HBSS fraction. This cell preparation containing a highly purified colonic epithelial cell fraction was spun down and resuspended in 1 ml/colon of ice-cold RIPA buffer containing protease inhibitors (Complete Mini EDTA-free, Roche, Indianapolis, Ind.). Cells were lysed for 30 min at 4° C. and lysates were spun for 30 min at maximum speed at 4° C. using a tabletop centrifuge (Eppendorf, Hauppauge, N.Y.). 500 ml of cleared lysates were immunoprecipated with 1 mg anti-NLRP6 antibody (clone E20, Santa Cruz Biotechnologies, Santa Cruz, Calif.) and 25 ml of Protein G agarose (Invitrogen, Grand Island, N.Y.) for 12 hours at 4° C. Agarose beads were washed five times with RIPA buffer and finally bound proteins eluted by boiling in loading buffer. Samples were separated on 10% TGX gels (Biorad, Hercules, Calif., Hercules, Calif.) and transferred onto PVDF membranes. Western blot analysis was performed using a anti-NLRP6 polyclonal antibody (clone E20, Santa Cruz Biotechnologies, Santa Cruz, Calif.) and anti-Goat-HRP (Zymax, Escondido, Calif.).

Isolation of Colonic CD45+ Cells and FACS Analysis and Sorting Colons were excised and washed thoroughly by flushing several times with PBS. They were opened longitudinally, transferred into HBSS+2 mMEDTA, and shaken for 20 min at 37° C. Subsequently, colons were washed 3 times with PBS. The lamina propia was then digested for 45 min at 37° C. using “digest solution” (DMEM containing 2% FCS, 2.5 mg/ml Collagenase, 1 mg/ml DNasel, and 1 mM DTT). Single cell suspensions were obtained by grinding through a 100 mm cell strainer (Fisher Scientific, Pittsburgh, Pa., Pittsburgh, Pa.). For FACS analysis, single suspensions were stained with anti-CD11c, anti-CD11b, anti-MHC class II, anti-TCR-beta, anti-TCR-gamma/delta, anti-B220, anti-NK1.1, and anti-CD45.2 (all from BD Biosciences, San Jose, Calif., San Jose, Calif. or Biolegend, San Diego, Calif.) and analyzed on a BD LSR II. For FACS sorting, cells were stained with anti-mouse CD45.2-PacificBlue (Biolegend, San Diego, Calif.) and sorted twice iteratively on a BD FACS Aria to increase the purity of the positively sorted population.

### Gene Expression Analysis

Tissues were preserved in RNAlater solution (Ambion, Grand Island, N.Y.) and subsequently homogenized in Trizol reagent (Invitrogen, Grand Island, N.Y.). Cells subjected to FACS were resuspended in Trizol reagent. RNA was purified according to the manufacturer's instructions. One microgram of total RNA was used to generate cDNA (HighCapacity cDNA Reverse Transcription kit; Applied Biosystems, Carlsbad, Calif.). RealTime-PCR was performed using gene-specific primer/probe sets (Applied Biosystems, Carlsbad, Calif.) and Kapa Probe Fast qPCR kit (Kapa Biosystems, Woburn, Mass.) on a 7500 Fast Real Time PCR instrument (Applied Biosystems, Carlsbad, Calif.). PCR conditions were 95° C. for 20 seconds, followed by 40 cycles of 95° C. for 3 seconds and 60° C. for 30 seconds. Data were analyzed using the Sequence Detection Software according the deltaCt method with hprtl serving as the reference housekeeping gene.

### Colonic Explants

Two 0.5 cm long pieces from the proximal colon were removed from a given animal, rinsed with PBS, and weighed. The tissue explants were cultured for 24 hours in DMEM medium containing 10% FBS, L-glutamine, penicillin, and streptomycin at 37° C. Culture medium was removed, centrifuged (1200×g for 7 min at 4° C.), and the resulting supernatant stored in aliquots at −20° C.

### ELISA and Multiplex Analysis

Concentrations of cytokines and immunoglobulins in the serum or culture supernatants were measured using the following commercial ELISA kits: CCL5 (PeproTech, Rocky Hill, N.J.); IL-18 (MBL); IgG1, IgG2c (BD Biosciences, San Jose, Calif.), IgA, IgM (Bethyl Laboratories, Montgomery, Tex.) according to manufacturer's instruction. Multiplex analysis was performed using the Bioplex 23-Plex Panel (Biorad, Hercules, Calif.) according to the manufacturer's instructions.

16S rRNA Analyses

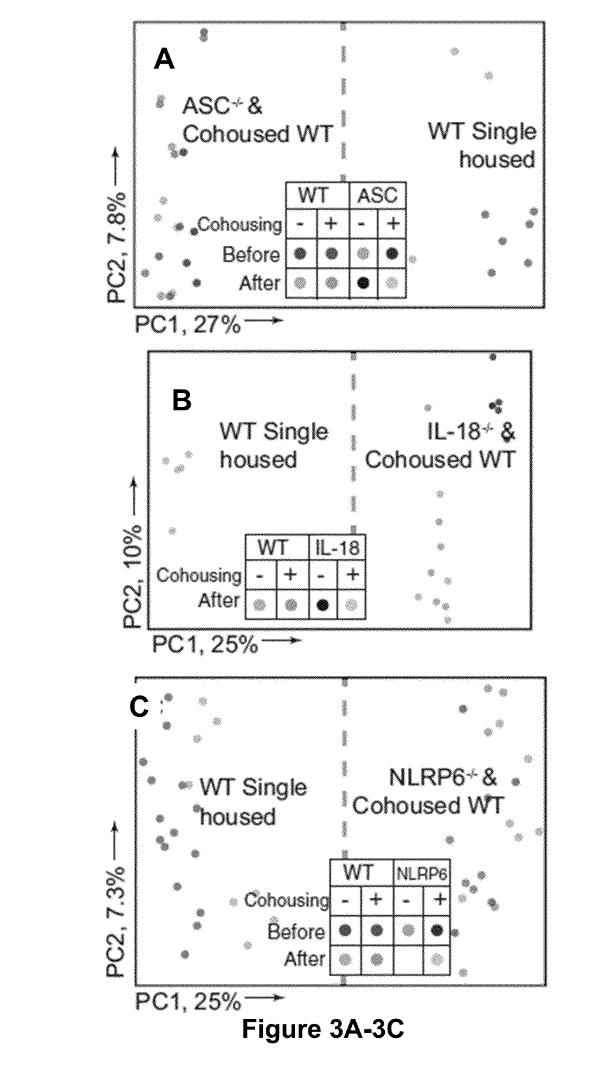

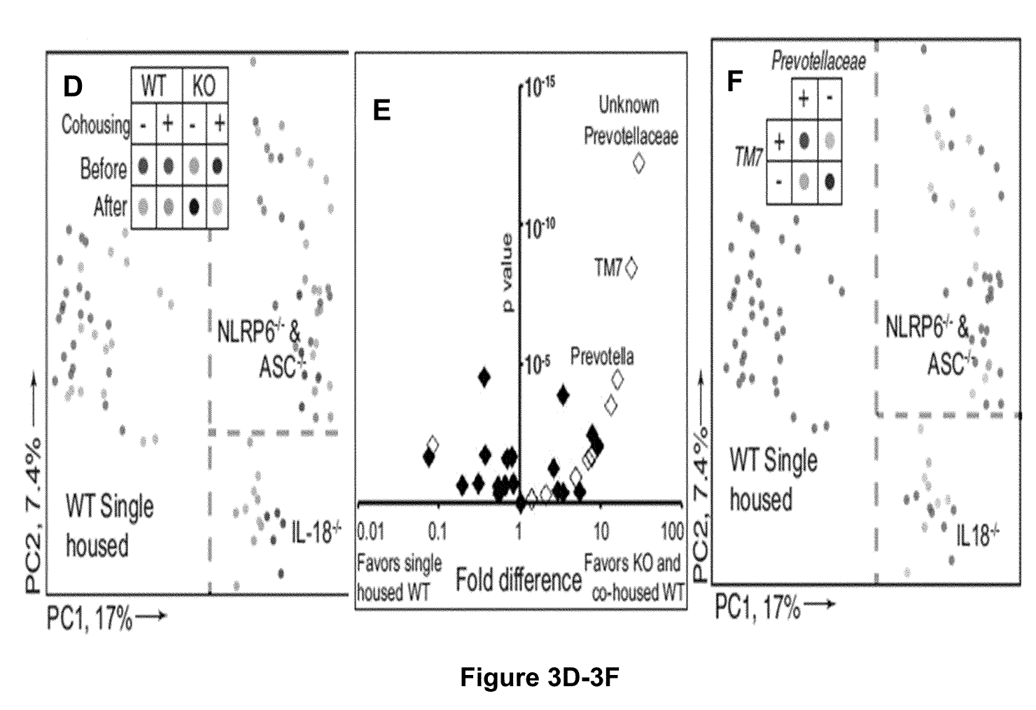

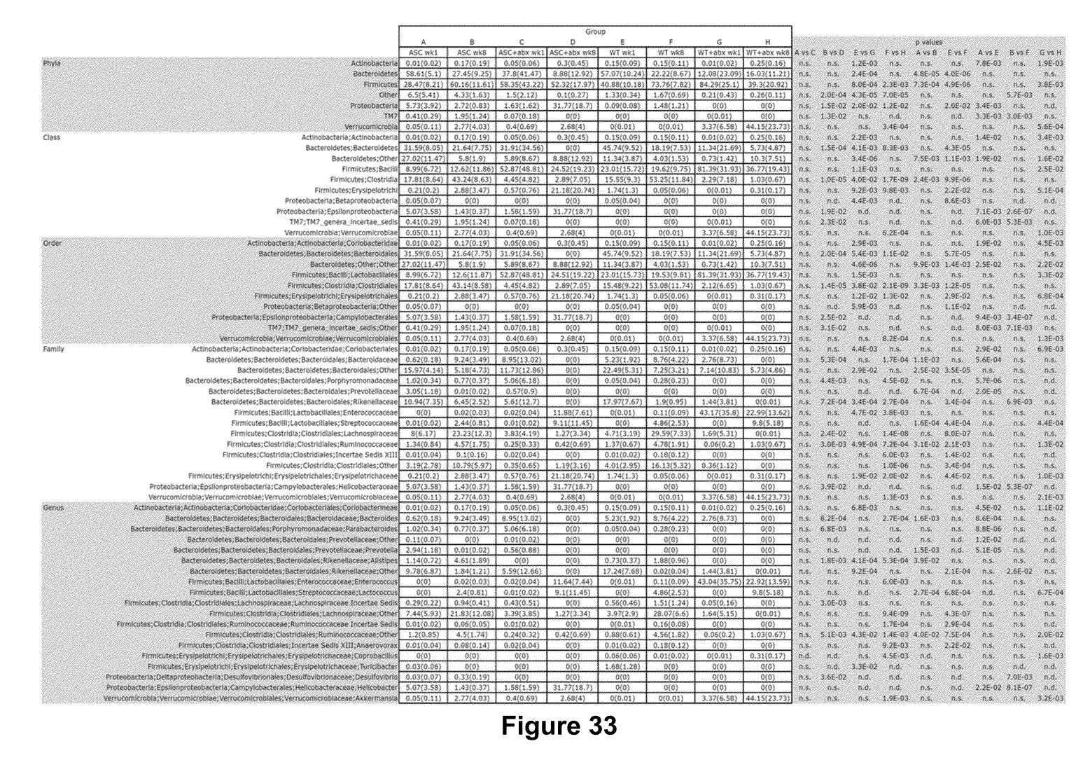

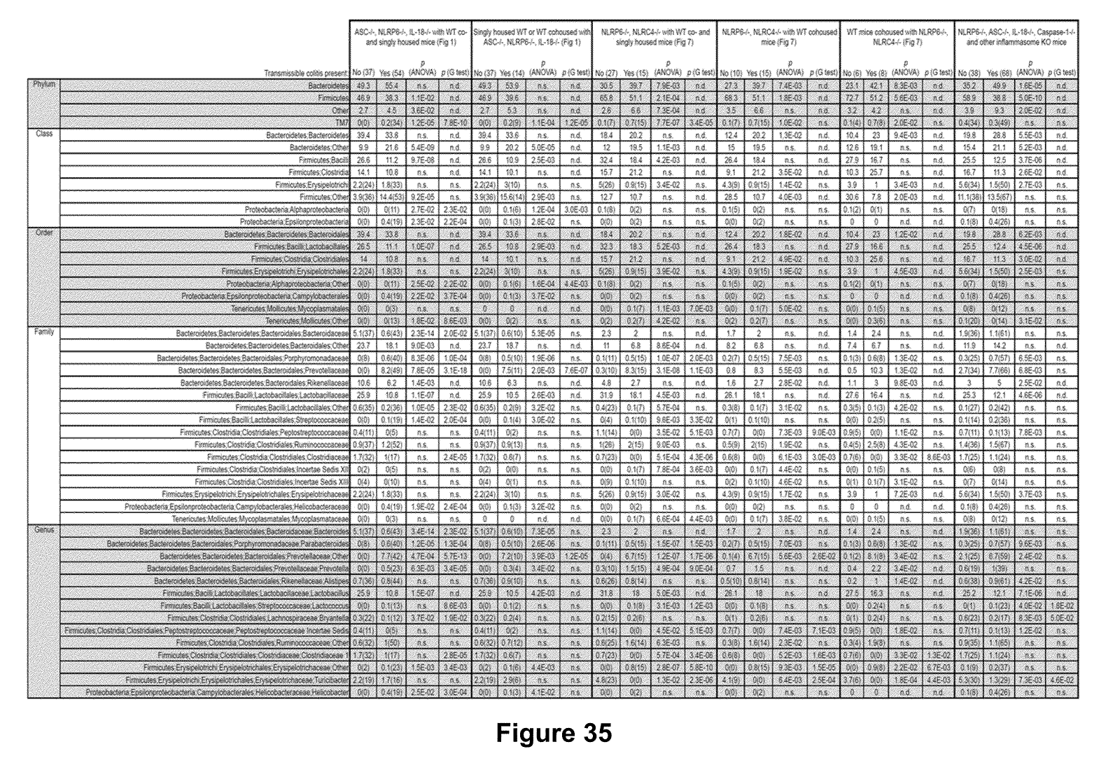

Aliquots of frozen fecal samples (n=212) were processed for DNA isolation using a previously validated protocol (Turnbaugh et al., 2009, Nature 457:480-484). An aliquot of the purified fecal DNA was used for PCR amplification and sequencing of bacterial 16S rRNA genes. ˜365 bp amplicons, spanning variable region 2 (V2) of the 16S rRNA gene were generated by using (i) modified primer 8F (5′-CCATCTCATCCCTGCGTGTCTCCGACTCAGTCAGAGTTTGATCCTGGCTCA G-3′; SEQ ID NO: 1) which consists of 454 Titanium primer B (underlined) and the universal bacterial primer 8F (italics) and (ii) modified primer 338R (5′-CCTATCCCCTGTGTGCCTTGGCAGTCTCAGNNNNNNNNCATGCTGCCTCCC GTAGGAGT-3′; SEQ ID NO:2) which contains 454 Titanium primer A (underlined), a sample specific, error correcting 8-mer barcode (N's), and the bacterial primer 338R (italics). Three replicate polymerase chain reactions were performed for each fecal DNA sample. The reactions were subsequently pooled, DNA was quantified (Picogreen), pooled in an equimolar ratio, purified (Ampure magnetic purification beads) and used for multiplex 454 pyrosequencing (Titanium chemistry). Reads were initially processed using the QIIME (Quantitative Insights Into Microbial Ecology) analysis pipeline (Caporaso et al., 2010, Nat. Methods 7:335-336): fasta, quality files and a mapping file indicating the barcode sequence corresponding to each sample were used as inputs. The QIIME pipeline takes this input information and splits reads by samples according to the barcode, performs taxonomical classification using the RDP-classifier, builds a de-novo taxonomic tree of the sequences based on sequence similarity, and creates a sample x OTUs table that can be used, together with the tree, for calculating beta diversity. After chimera removal, the dataset consisted of 747,125 sequences (average number of reads/fecal sample, 3,524±1023 (SD); average read length, 361 nt). Sequences sharing R 97% nucleotide sequence identity in the V2 region were binned into operation taxonomic units (97% ID OTUs) using uclust, chimeric sequences were removed using ChimeraSlayer (Haas et al., 2011). Note that only 97% ID OTUs found 10 or more times among the 212 samples in the analyses were considered. The OTU table was rarefied to 100 reads per sample to normalize the depth of sequencing per sample. (The OTU table used for our analyses is accessible at Data S1. A key describing the genotype and housing of each mouse (FIG. 35) shown in FIG. 3 can be found at FIG. 33).

qPCR assays used previously reported primer pairs that target Prevotellaceae (5′-CCAGCCAAGTAGCGTGCA-3′; SEQ ID NO:3) and 5′-TGGACCTTCCGTATTACC-3′; SEQ ID NO:4) (Dalwai et al., 2007), TM7 (5′-GCAACTCTTTACGCCCAGT-3′; SEQ ID NO:5 and 5′-GAGAGGATGATCAGCCAG-3′; SEQ ID NO:6) and Bacteria (5′-AGAGTTTGATCCTGGCTC-3′; SEQ ID NO:7 and 5′-TGCTGCCTCCCGTAGGAGT-3′; SEQ ID NO:8) (Bjoersdorff et al., 2002, Clin. Diagn. Lab. Immunol. 9:341-343). The PCR mix contained 5 ml of the sample DNA solution, 5 pmol of each primer, 0.2 ml of bacteria-specific probes and 5 ml of Universal qPCR mix (Kapa Biosystems, Woburn, Mass.). PCR conditions were 95° C. for 120 seconds, followed by 40 cycles of 95° C. for 3 seconds and 64° C. for 30 seconds. Data were analyzed using the Sequence Detection Software according the deltaCt method by normalizing tested bacterial species to total bacteria for each sample.

To quantify attached bacteria enriched in the crypts, colons were excised and thoroughly washed five times with 10 ml of PBS to remove all fecal contents. Tissues were then homogenized in Trizol and genomic DNA was purified according to the manufacturer's instructions.

### Statistical Analysis

Data are expressed as mean±SEM. Differences were analyzed by Student's t test and ANOVA and post-hoc analysis for multiple group comparison. P values≦0.05 were considered significant.

### Accession Numbers

16S rRNA data sets have been deposited in MG-RAST under accession number qiime:654.

The results of the experiments are now described.

ASC^{-/-}-Deficient Mice Develop Severe DSS Colitis that is Transferable to Cohoused WT Mice

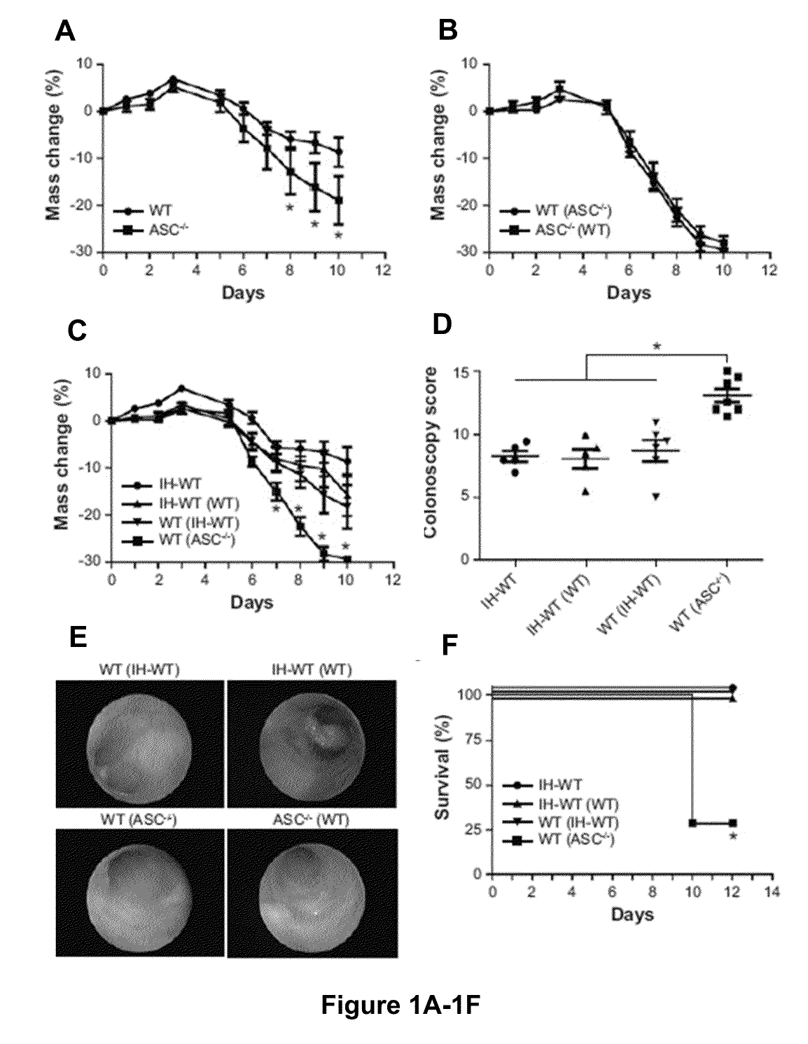

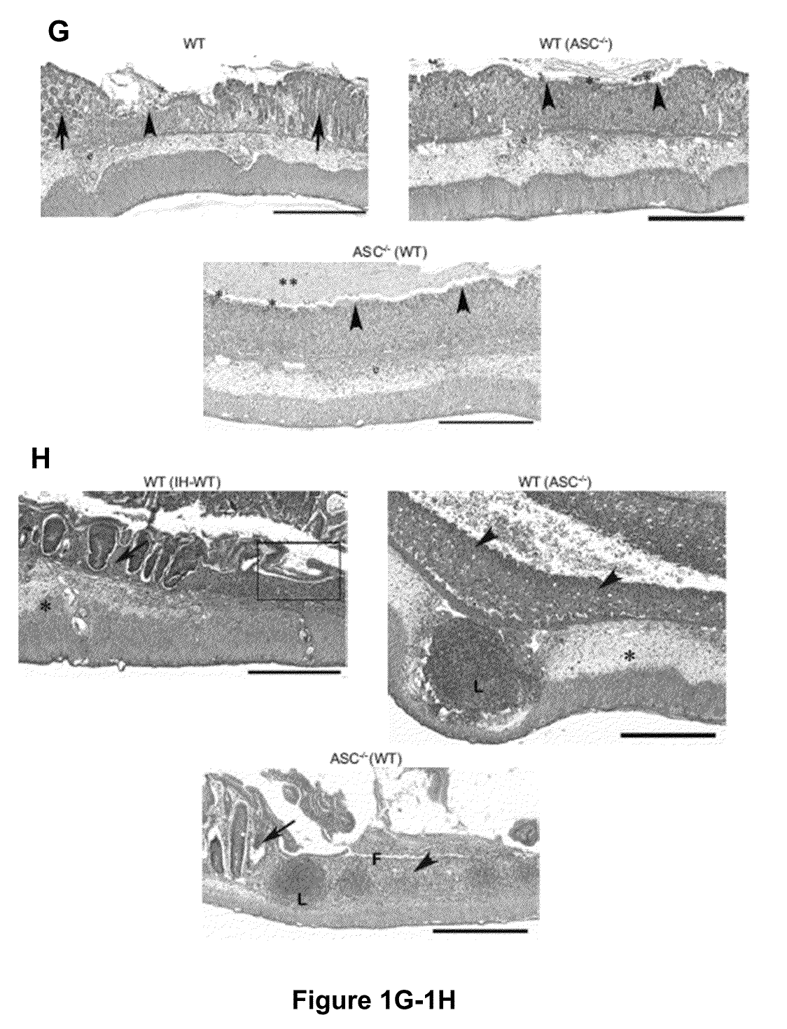

To characterize possible links between inflammasome function and homeostasis achieved between the innate immune system and the gut microbiota, mice were studied that are deficient in ASC. A more severe colitis developed after dextran sodium sulfate (DSS) administration to single-housed ASC^{-/-} mice than in wild-type (WT) mice purchased from a commercial vendor (National Cancer Institute, NCI) (FIG. 1A). Remarkably, cohousing of adult ASC^{-/-} mice with age-matched WT mice for 4 weeks prior to induction of DSS colitis resulted in development of comparably severe DSS-induced colitis in ASC^{-/-} as well as cohoused WT mice (the latter are designated “WT(ASC^{-/-})” in FIG. 1B).

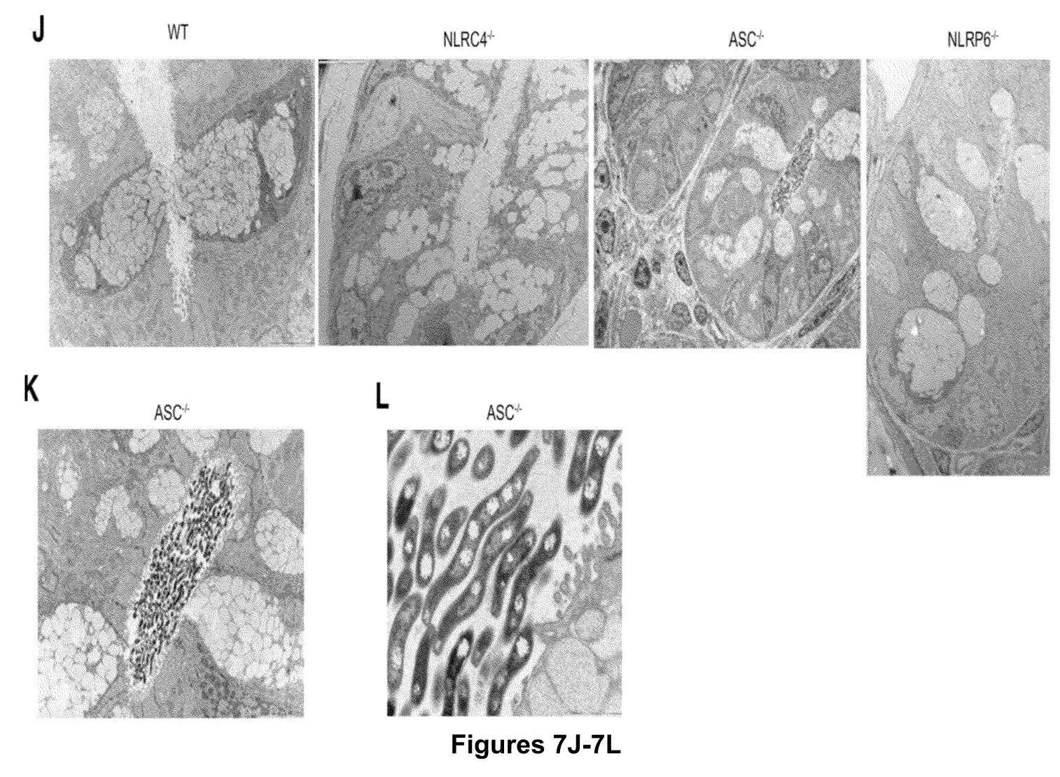

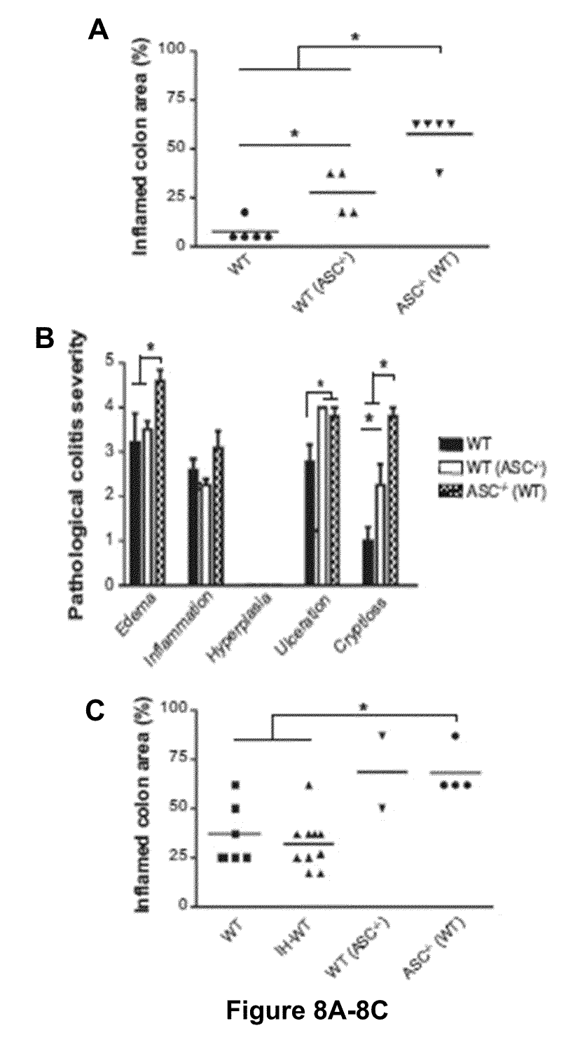

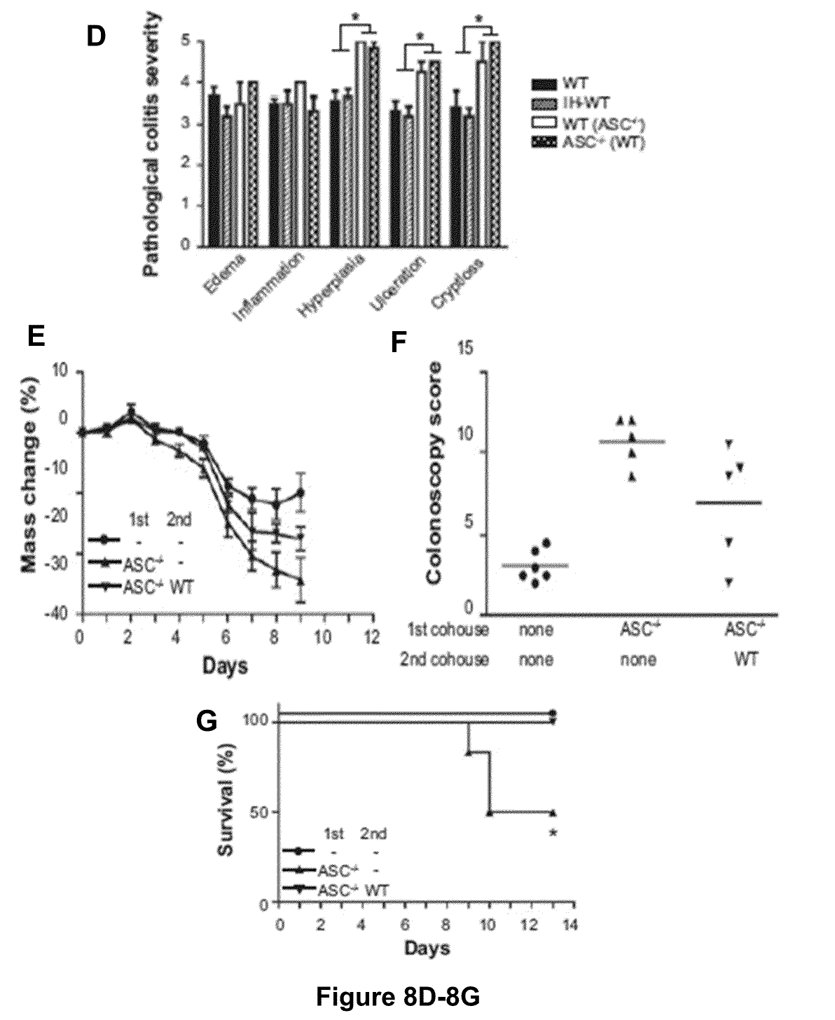

To assess the possibility that differences in colitis severity observed between groups of single-housed ASC^{-/-} and WT mice were indeed driven by differences in their intestinal microbiota, WT mice were cohoused for 4 weeks with either ASC^{-/-} mice (WT(ASC^{-/-})) or WT mice that had been bred in a vivarium for more than ten generations (in-house mice (IH-WT), WT(IH-WT)). The severity of DSS-induced colitis was similar among NCI-WT, IH-WT, and WT(IH-WT) as well as IH-WT(WT) as judged by weight loss (FIG. 1C), colitis severity score (defined by colonoscopy) (FIGS. 1D and 1E), and survival (FIG. 1F). In contrast, WT(ASC^{-/-}) and ASC^{-/-} mice were characterized by an equally increased severity of disease compared to these other groups at both early and late stages (FIGS. 1C-1H and FIGS. 8A-8D).

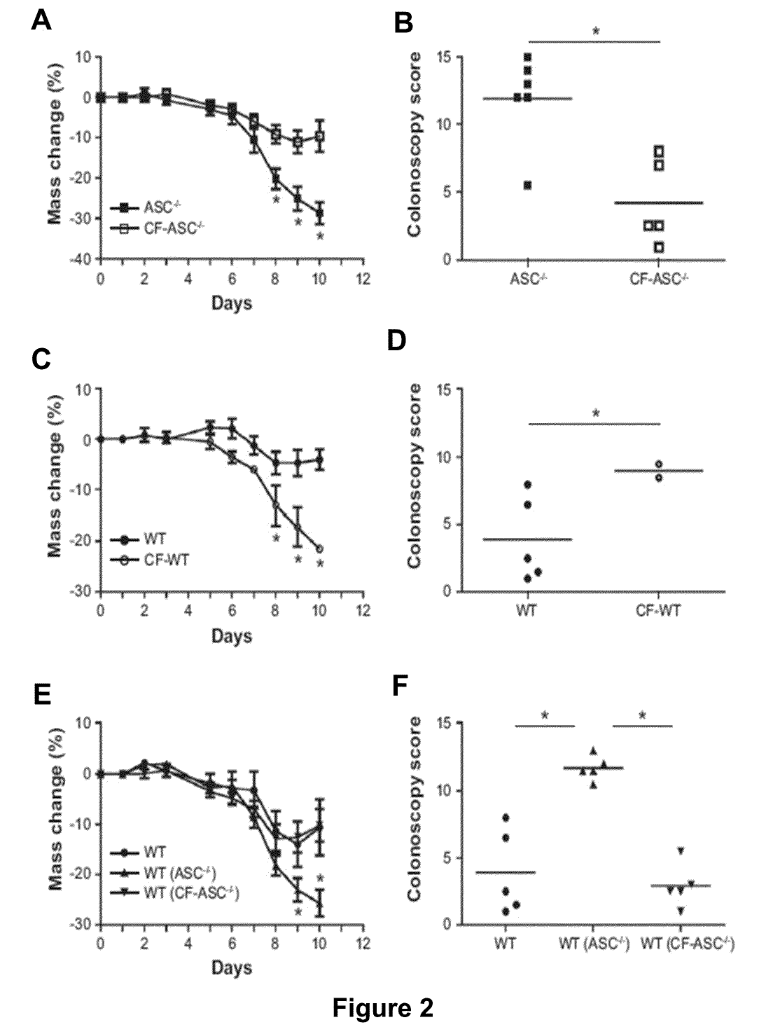

To further establish the role of the intestinal microbiota, cross-fostering experiments were performed. Newborn ASC^{-/-} mice cross-fostered (CF) at birth with in-house WT mothers (CF-ASC^{-/-}) exhibited milder colitis compared to noncross-fostered ASC^{-/-} mice (FIGS. 2A and 2B). In contrast, newborn WT mice cross-fostered with ASC^{-/-} mothers (CF-WT) developed severe colitis in comparison to noncross-fostered WT mice (FIGS. 2C and 2D). Moreover, CF-ASC^{-/-} mice were no longer able to transmit enhanced colitis to cohoused WT mice (FIGS. 2E and 2F).

Separation of cohoused WT mice from ASC^{-/-} mice and subsequent housing with naive WT mice resulted in a gradual partial reduction in colitis severity compared to WT(ASC^{-/-}) mice that were not exposed to a WT microbiota (FIGS. 8E-8G). Together, these results demonstrated that the ASC^{-/-} microbiota is a dominant colitogenic factor, transmissible early in life to WT mice, and that this colitogenic activity is sustainable in recipient mice for prolonged periods of time. Nonetheless, exposure of an established transferred ASC^{-/-} derived microbiota in a WT mouse to WT microbiota ameliorated its colitogenic potential, suggesting that the latter community can displace the former and diminish its disease-promoting properties in WT mice.

Culture-independent methods were subsequently employed to compare the gut microbial communities. PCR was used to amplify variable region 2 (V2) of bacterial 16S rRNA genes present in fecal samples collected from ASC^{-/-} and WT mice just prior to and 28 days following cohousing. The amplicons generated were subjected to multiplex pyrosequencing, and the resulting chimera-checked and filtered data sets were compared using UniFrac (mean of 3524±1023 [SD] 16S rRNA reads/sample; as described above), FIG. 3A shows a clear difference in fecal bacterial phylogenetic architecture in WT versus ASC^{-/-} mice. Moreover, after 4 weeks of cohousing, the fecal bacterial communities of WT(ASC^{-/-}) mice clustered together with communities from their ASC^{-/-} cagemates. In addition, the bacterial component of the fecal microbiota of these cohoused ASC^{-/-} mice was similar to ASC^{-/-} mice that never had been cohoused.

NLRP6-Deficiency Produced a Microbiota-Mediated Phenotype that Resembled that of ASC Deficiency

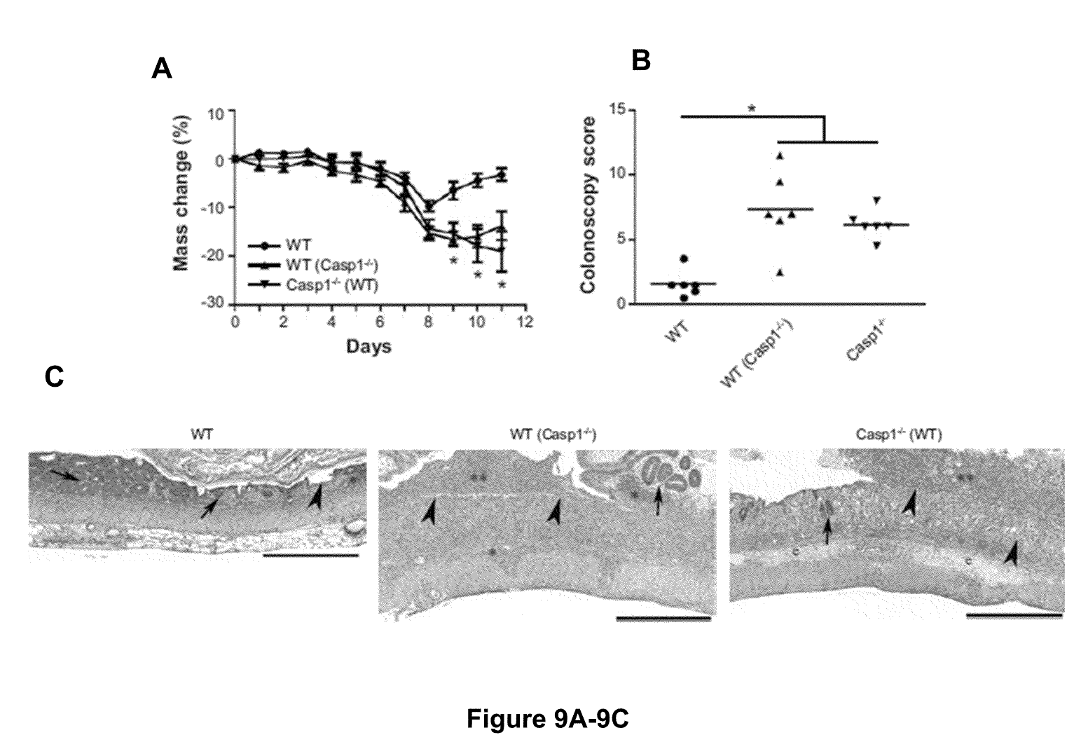

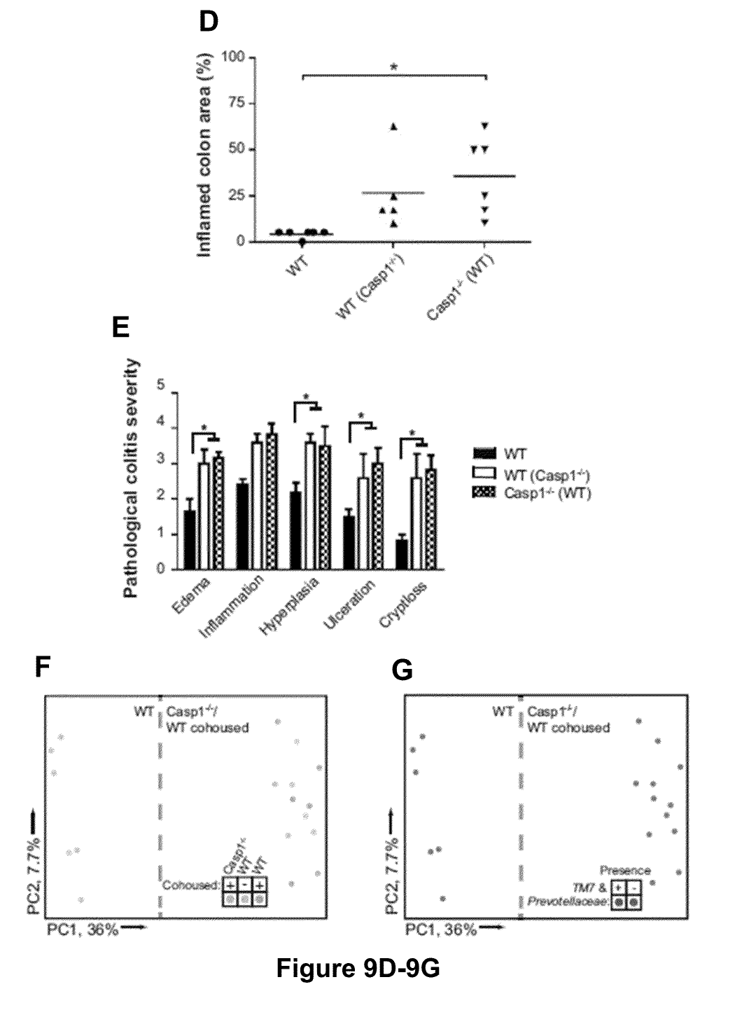

To assess whether ASC's fiaction as adaptor protein for inflammasome formation is linked to the changes in gut bacterial community structure and function observed, WT mice were cohoused with caspase-1^{-/-} mice, and these also exhibited more severe DSS-induced colitis compared to single-housed WT mice (FIGS. 9A-9E). Similar to WT mice cohoused with ASC^{-/-} mice, WT mice cohoused with caspase-1^{-/-} mice evolved their intestinal bacterial communities to a phylogenetic configuration that was very similar to that of their caspase-1^{-/-} cagemates (FIG. 9F). These results pointed to the involvement of an inflammasome in this phenotype.

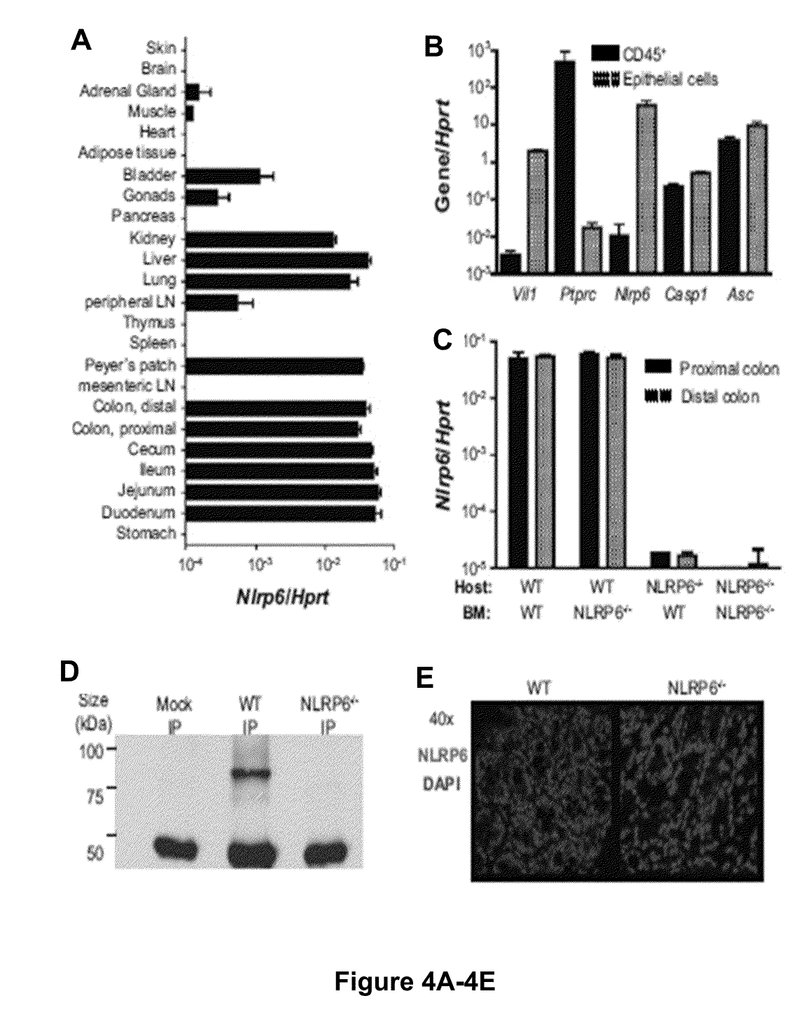

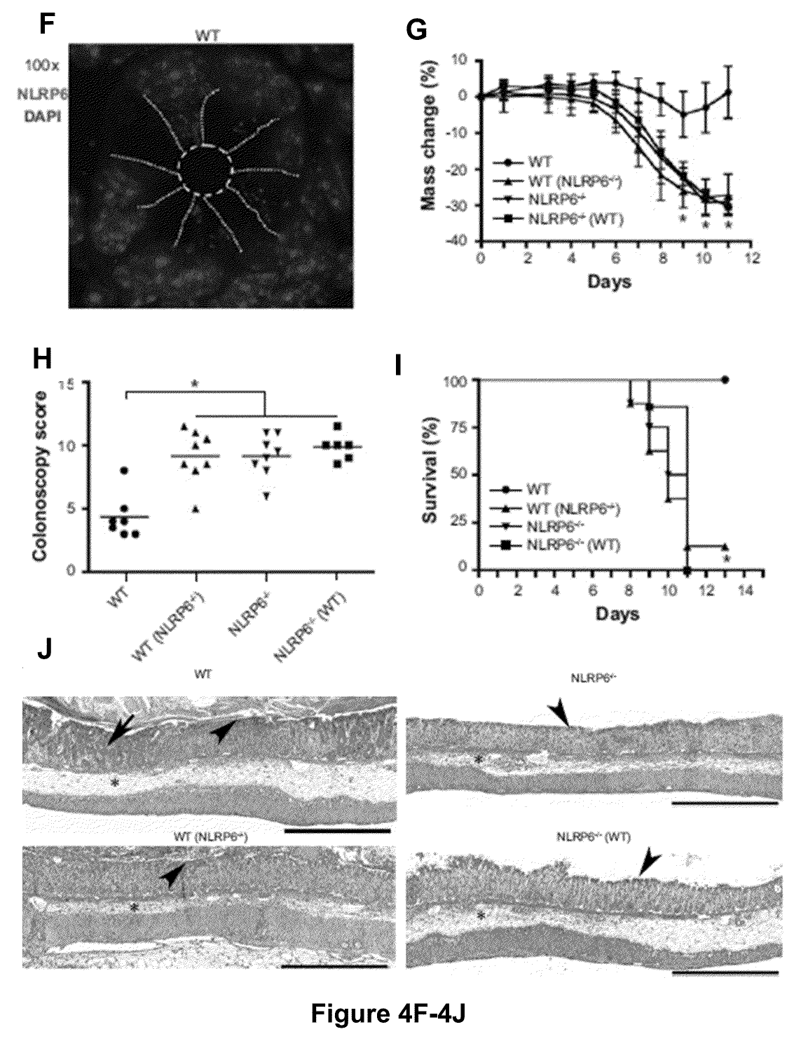

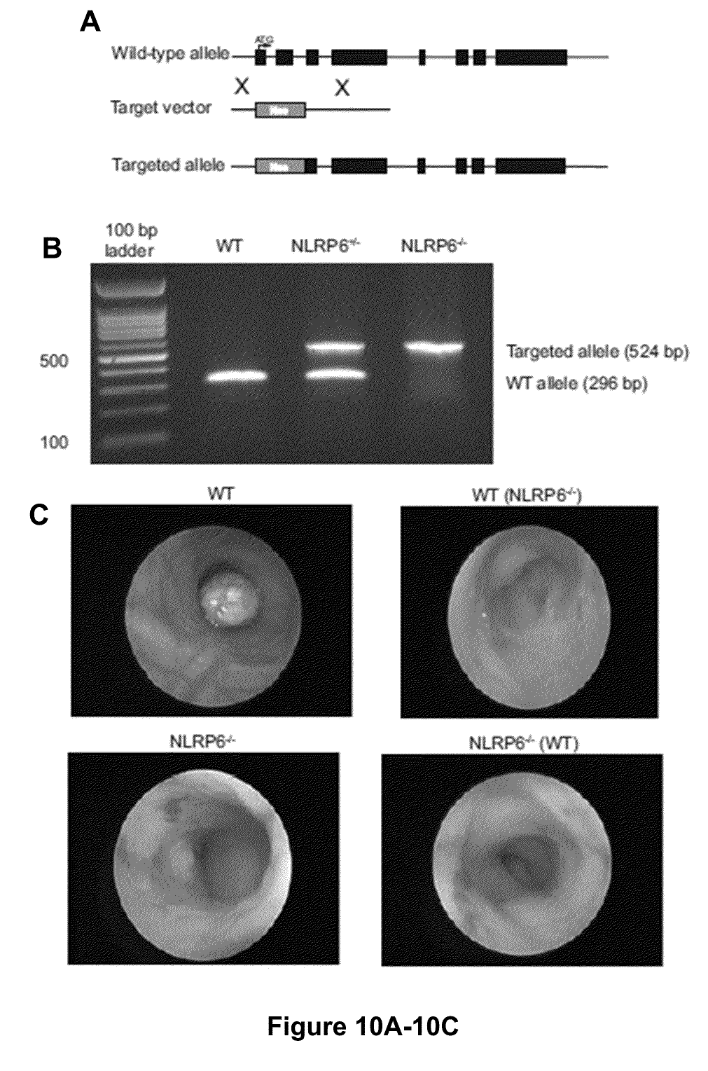

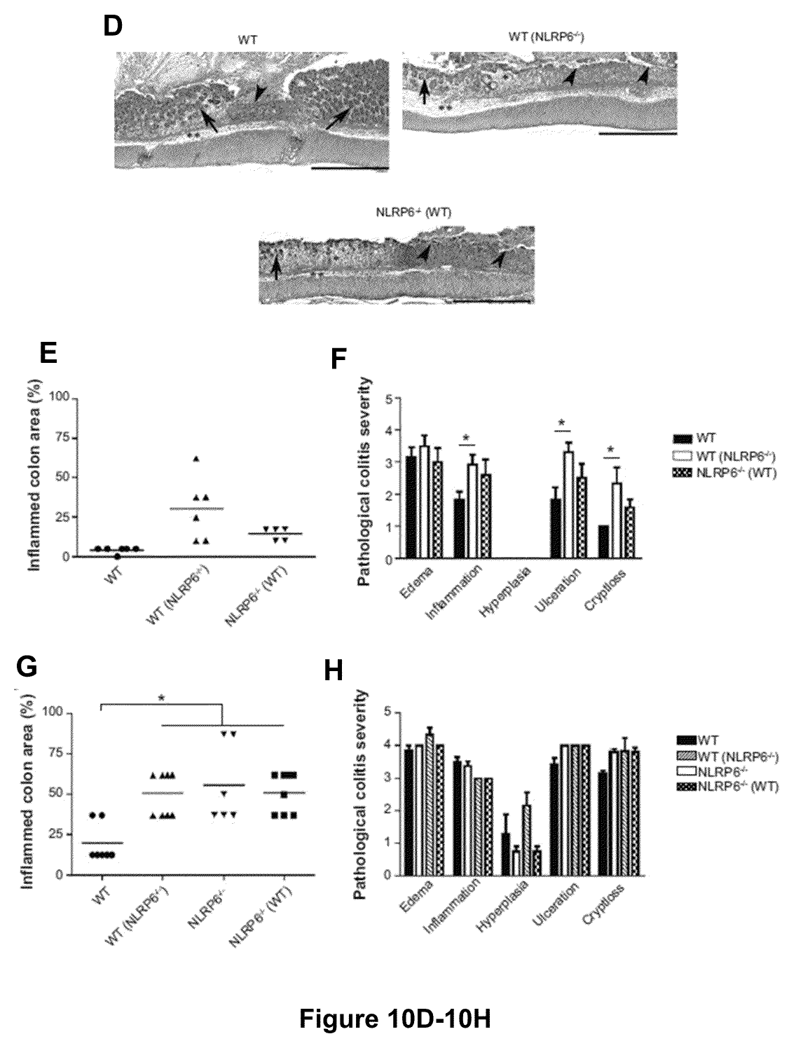

Next, the NLR(s) upstream of ASC and caspase-1 leading to the phenotype were identified. qRT-PCR analysis of 24 tissues in WT mice revealed that NLRP6, which forms an ASC-dependent inflammasome (Grenier et al., 2002, FEBS Lett. 530:73-78), is most highly expressed in the gastrointestinal tract and at lower levels in lung, kidney, and liver (FIG. 4A). Further, RNA prepared from colonic epithelium and sorted colonic CD45⁺ hematopoietic cells was isolated and it was found that ASC and caspase-1 are highly expressed in both compartments. NLRP6 expression, in contrast, was essentially limited to the epithelial compartment (FIG. 4B). Indeed, in bone marrow transfer experiments, NLRP6 was almost undetectable in NLRP6^{-/-} mice (FIGS. 10A and 10B) receiving WT bone marrow (FIG. 4C). Follow-up immunoprecipitation (FIG. 4D) and immunofluorescence assays (FIGS. 4E and 4F) both showed that NLRP6 protein was expressed in primary colonic epithelial cells of WT mice, where it mainly appeared within speckled cytoplasmic aggregates, whereas it was absent in NLRP6^{-/-} mice.

WT and NLRP6^{-/-} mice were then single housed or cohoused for 4 weeks, followed by exposure to DSS. Single-housed NLRP6^{-/-} mice developed more severe colitis compared to single-housed WT mice (FIGS. 4G-4J). The more severe colitis phenotype was transferable to cohoused WT mice (WT(NLRP6^{-/-})) (FIGS. 4G-4J and FIGS. 10C-10G). 16S rRNA analysis of fecal bacterial communities demonstrated a clear difference in the bacterial community structure between single-housed adult WT mice versus age-matched WT mice cohoused for 4 weeks with NLRP6-deficient mice (FIG. 3C). Fecal bacterial communities of WT mice clustered together with communities from their NLRP6^{-/-} cagemates whose microbiota in turn was similar to NLRP6^{-/-} mice that never had been cohoused (FIG. 3C).

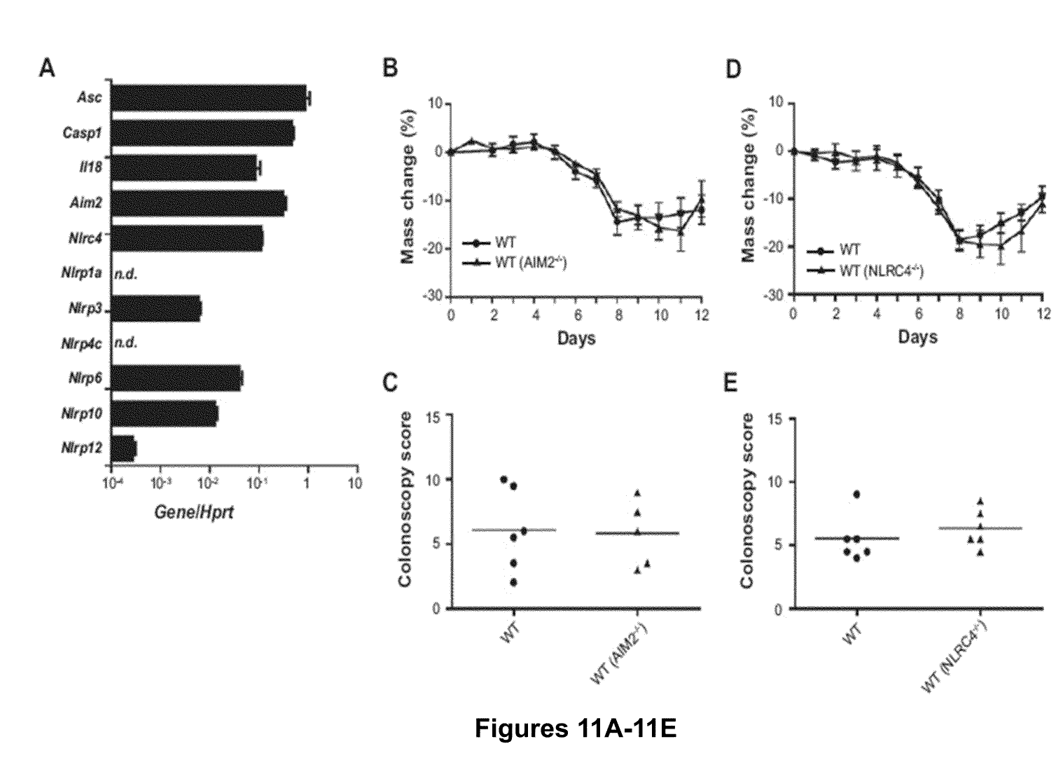

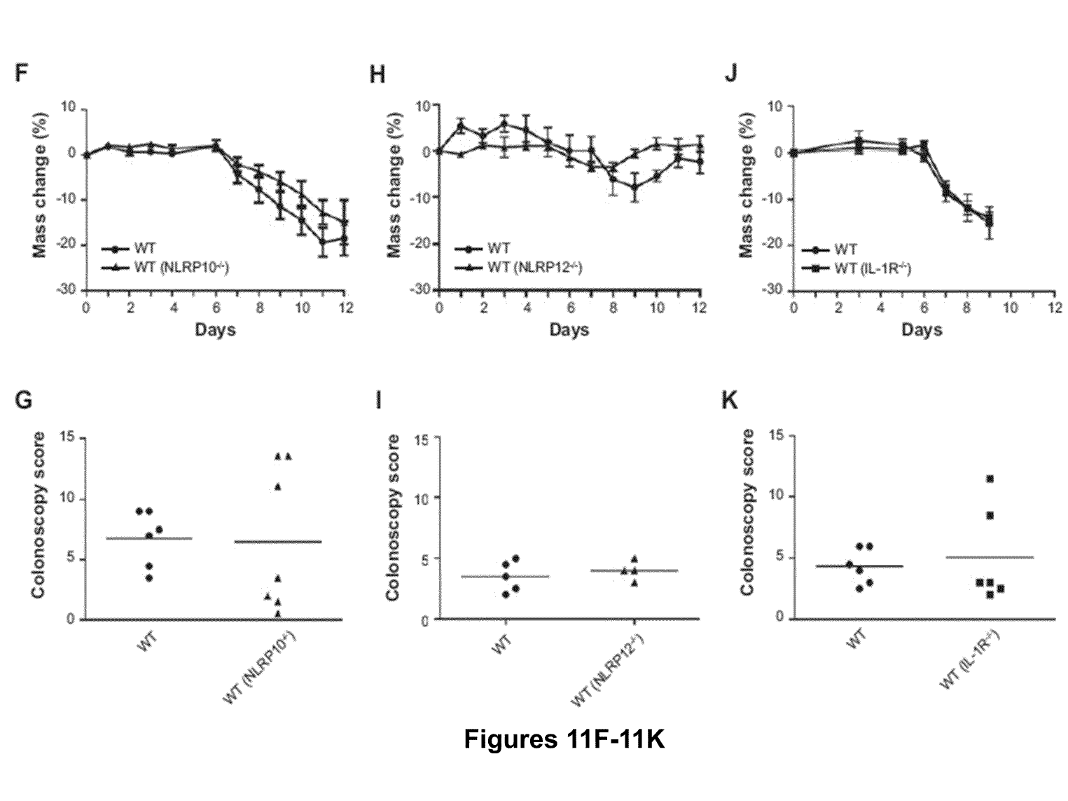

To ascertain the specificity of this phenotype, WT mice were cohoused with mice that lacked other NLR family members and inflammasome-forming protein AIM2, all shown by qRT-PCR analysis to be expressed in the colon (FIG. 11A) (Kufer et al., 2011, Nat. Immunol. 12:121-128; Schroder et al., 2010, Cell 140:821-832). Adult, conventionally raised, specific pathogen-free knockout mice were either obtained from the same source as NLRP6^{-/-} mice (Millenium, NLRP3^{-/-}, NLRC4^{-/-}, NLRP12^{-/-}), (NLRP10^{-/-}), or obtained firom other laboratories (AIM2^{-/-}, K Fitzgerald, U. Massachusetts). NLRP3^{-/-} mice cohoused with WT mice for 4 weeks featured attenuated colitis as compared to their WT cagemates and mild transferability of colitis, consistent with the explanation that NLRP3's major effect in this system is negative regulation of the inflammatory process itself. Importantly, none of the other above mentioned mouse strains transferred microbiota with increased colitogenic properties to WT mice upon cohousing (FIGS. 11B-11I). Likewise, 16S rRNA analysis of these strains revealed a distinct configuration of their microbiota population as compared to NLRP6 inflammasome-deficient mice (FIG. 35). Together, these findings indicate that NLRP6 forms an intestinal epithelial inflammasome that regulates functional properties of the microbiota and that loss of NLRP6 and the known inflammasome constituents, ASC and caspase-1, leads to the specific development of a transmissible, more colitogenic microbiota.

Evidence that NLRP6 Affects the Gut Microbiota via IL-18

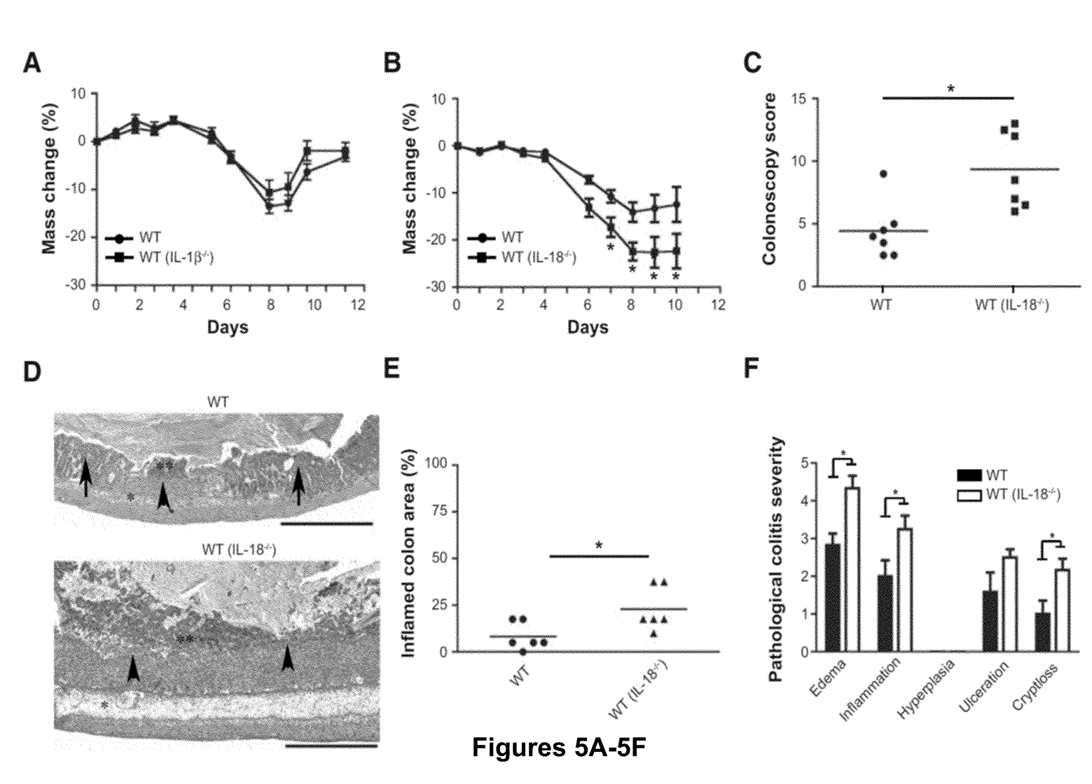

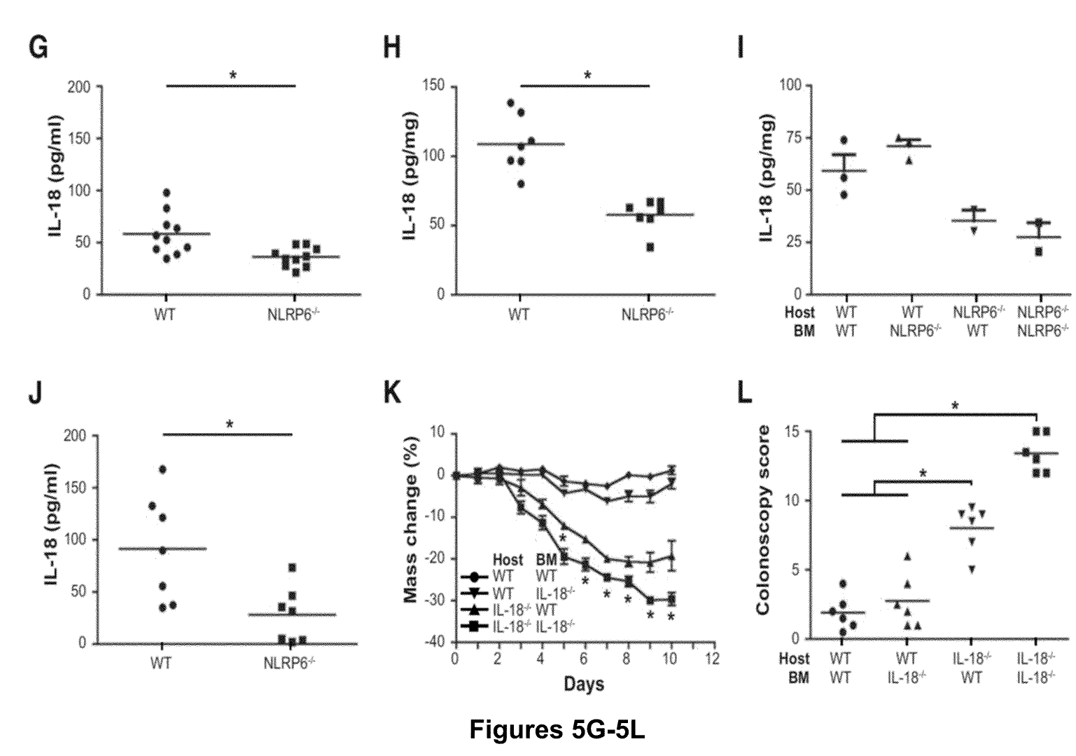

Activation of inflammasomes results in multiple downstream effects, including proteolytic cleavage of pro-IL-1β and pro-IL-18 to their active forms (Schroder et al., 2010, Cell 140:821-832). To test whether the effect of NLRP6 deficiency is mediated via IL-1β or IL-18 deficiency, adult WT mice were cohoused with either IL-1β^{-/-} (FIG. 5A) or IL-R^{-/-} mice (FIGS. 11J-11K). Cohousing WT mice with these strains did not result in any significant changes in the severity of DSS colitis compared to single-housed WT mice, excluding a major contribution of the IL-1 axis. In contrast, IL-18^{-/-} mice and, more importantly, WT mice cohoused with them exhibited a significant exacerbation of colitis severity, compared to single-housed WT mice (FIGS. 5B-5F).

In the steady state, single-housed NLRP6^{-/-} mice had significantly reduced serum levels of IL-18 compared to their WT counterparts and reduced production of this cytokine in their colonic explants (FIGS. 5G-5H). To study the relative contribution of hematopoietic and nonhematopoietic NLRP6 deficiency to this reduction in active IL-18, IL-18 protein levels were measured in colonic explants prepared from chimeric mice that had received bone marrow transplants from NLRP6^{-/-} or WT donors. Significantly lower IL-18 protein levels were noted only in explants prepared from mice with NLRP6 deficiency in the nonhematopoietic compartment (FIG. 5I). This result indicated that NLRP6 expressed in a nonhematopoietic component of the colon, likely the epithelium, is a major contributor to production of active IL-18. Furthermore, in contrast to WT mice, NLRP6^{-/-} mice failed to significantly upregulate IL-18^{-/-} in the serum and in tissue explants following induction of DSS colitis (FIG. 5J).

To study whether IL-18 production by nonhematopoietic cells is the major contributor to the microbiota-associated enhanced colitogenic phenotype, a bone marrow transfer experiment was performed using IL-18^{-/-} and WT mice as both recipients and donors. Indeed, mice that were deficient in IL-18 in the nonhematopoietic compartment exhibited more severe disease compared to mice that were sufficient for IL-18^{-/-} in the nonhematopoietic compartment (FIGS. 5K-5L). Bacterial 16S rDNA studies demonstrated that the fecal microbiota of WT mice exposed to IL-18^{-/-} mice changed its phylogenetic configuration to resemble that of IL-18^{-/-} cagemates (FIG. 3B). Interestingly, as seen in the PC2 axis in the PCoA plot of unweighted UniFrac distances, the fecal microbiota of ASC^{-/-} and NLRP6^{-/-} mice were distinct from IL-18^{-/-} mice, possibly reflecting the existence of additional NLRP6 inflammasome-mediated IL-18-independent mechanisms of microflora regulation (FIG. 3D). Together, these results concluded that the decrease in colonic epithelial IL-18 production in mice that are deficient in components of the NLRP6 inflammasome is critically involved in the enhanced colitogenic properties of the microbiota.

The Gut Microbiota from NLRP6 Inflammasome-Deficient Mice Induces CCL5 Production and Immune Cell Recruitment, Leading to Spontaneous Inflammation

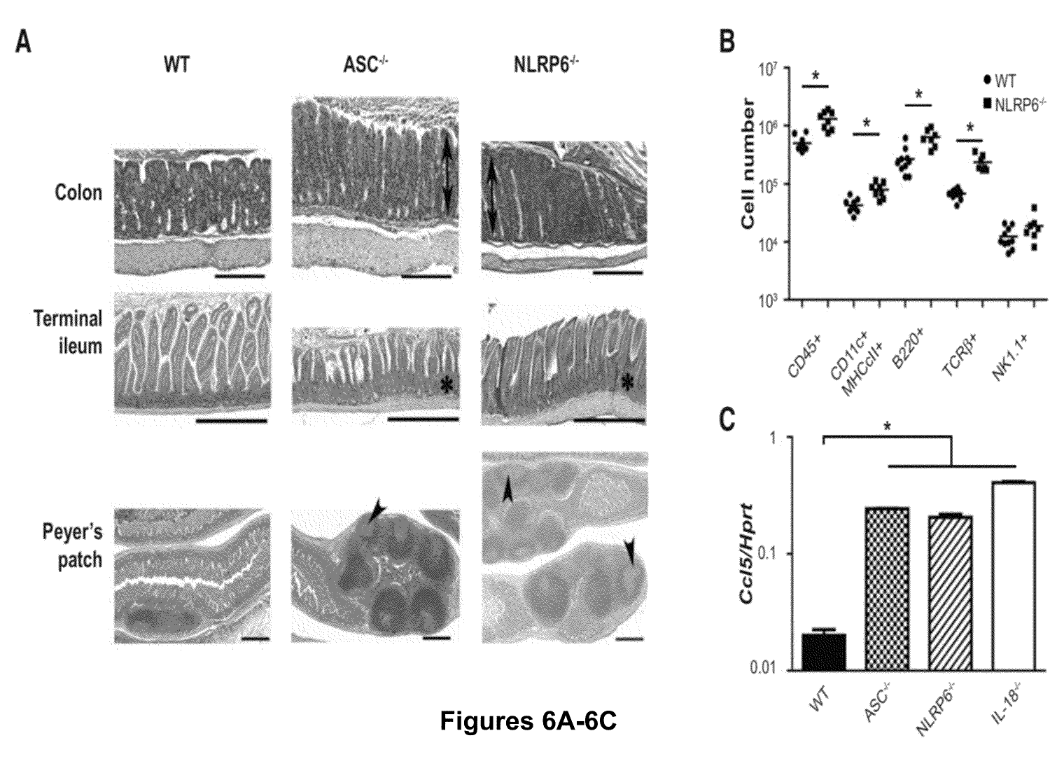

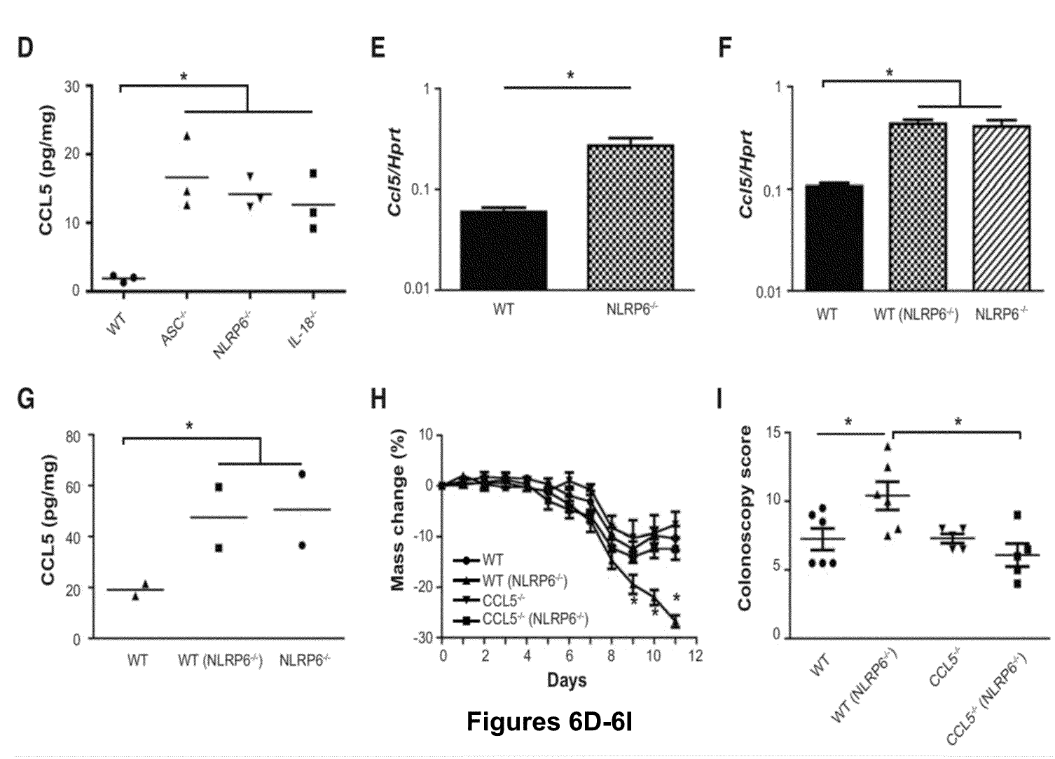

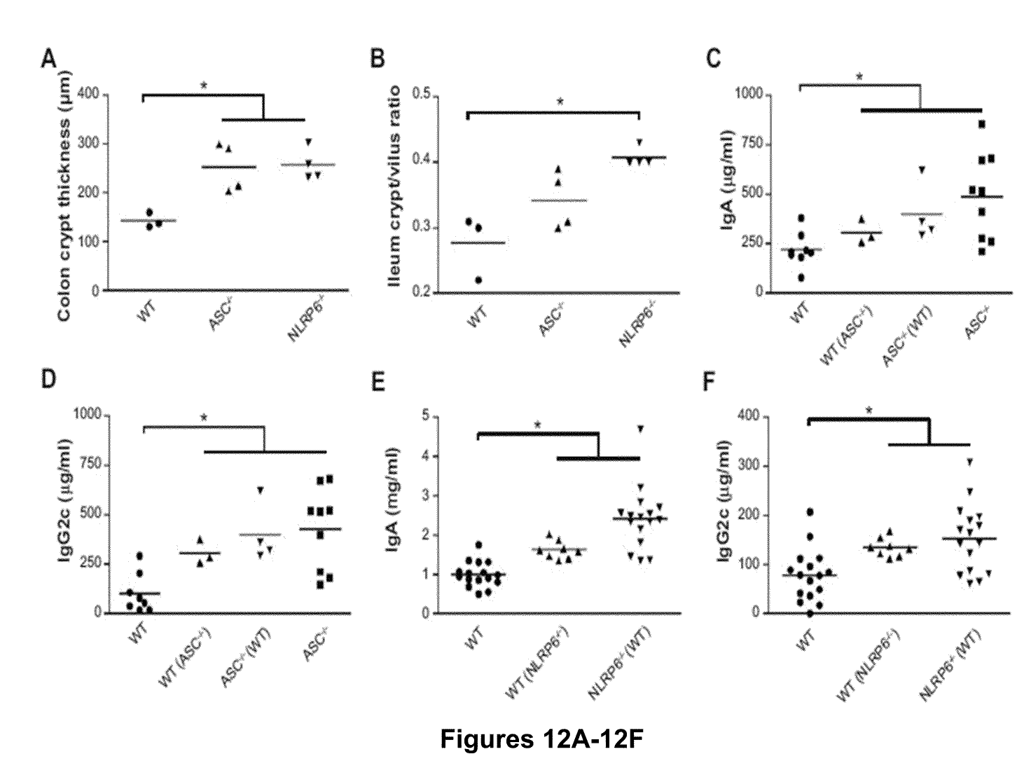

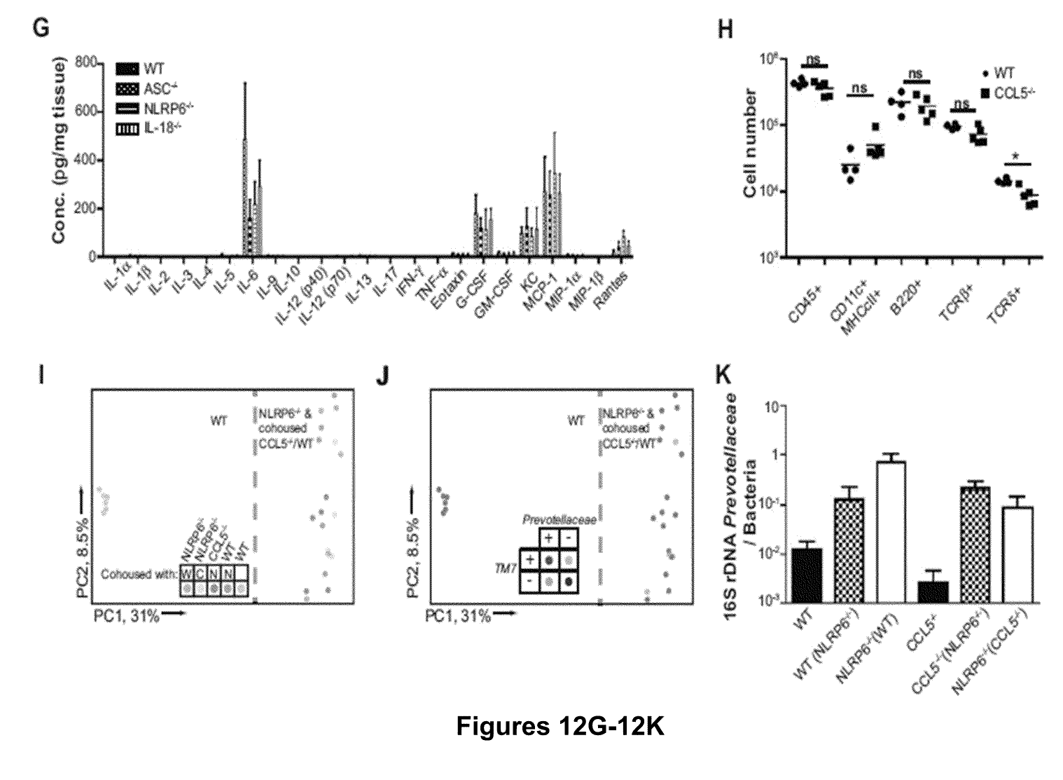

The intestines of untreated ASC^{-/-} and NLRP6^{-/-} mice were examined for signs of spontaneous pathological changes. The colons, terminal ileums, and Peyer's patches of ASC^{-/-} and NLRP6^{-/-} mice exhibited colonic crypt hyperplasia, changes in crypt-to-villus ratios in the terminal ileum, and enlargement of Peyer's patches with formation of germinal centers (FIG. 6A and FIGS. 12A-12B). NLRP6 inflammasome-deficient mice also had significantly elevated serum IgG2c and IgA levels, as did cohoused WT mice (FIGS. 12C-12F). In addition, significantly more CD45⁺ cells were recovered from colons of NLRP6^{-/-} mice compared to WT controls (FIG. 6B). Downstream effector mechanisms by which the altered microbiota could induce this immune cell infiltration were investigated. Multiplex analysis of cytokine and chemokine production by tissue explants (FIG. 12G), followed by validation at the RNA (FIG. 6C) and protein levels (FIG. 6D), indicated that CCL5 levels were significantly elevated in single-caged untreated ASC^{-/-}, NLRP6^{-/-}, and IL-18^{-/-} compared to WT mice. Furthermore, CCL5 mRNA upregulation was found to originate from epithelial cells (FIG. 6E). Moreover, CCL5 levels were induced in WT mice upon cohousing (FIGS. 6F-6G), showing that this property was specified by the microbiota and not the mutated inflammasome per se. Notably, in the steady state, CCL5^{-/-} mice and WT mice featured a comparable representation of immune subsets with the exception of slight reduction in γδ TCR⁺ lymphocytes, indicating that CCL5 is not generally required for immune cell recruitment to the colon (FIG. 12H).

To test the role of CCL5 in mediating the enhanced colitogenic properties of the NLRP6^{-/-} mouse microbiota, WT or CCL5^{-/-} mice were cohoused with NLRP6^{-/-} mice for 4 weeks. DSS colitis was subsequently induced and comparable colitis severity was found between single-housed WT and CCL5^{-/-} mice (FIGS. 6H and 6I). However, upon cohousing, WT(NLRP6^{-/-}) mice had significantly worse DSS-induced colitis compared to CCL5^{-/-}(NLRP6^{-/-}) mice, despite comparable acquisition of the NLRP6^{-/-} colitogenic flora (FIG. 12I). These findings support the notion that CCL5 upregulation in response to the altered microbiota is responsible for the exacerbation of colitis that occurs in WT mice cohoused with NLRP6 inflammasome-deficient mice.

Identification of Bacterial Ph lotypes that Are Markedly Expanded in Both NLRP6 Inflammasome-Deficient Mice and in Cohoused WT Mice

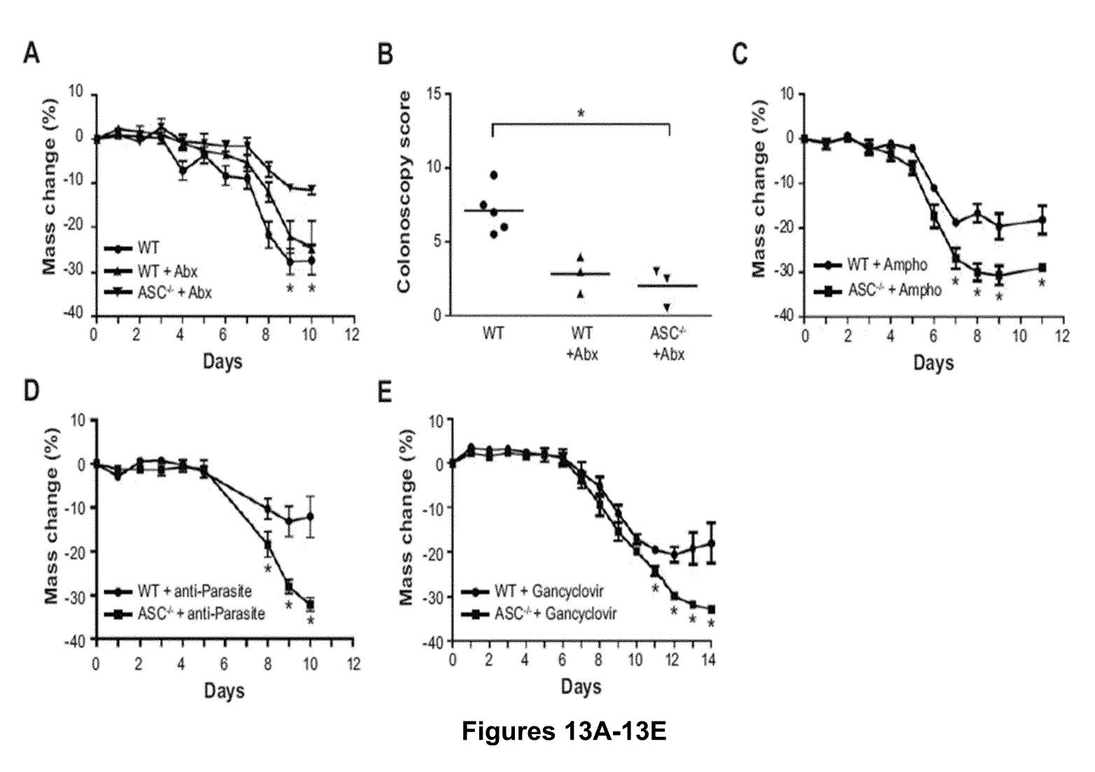

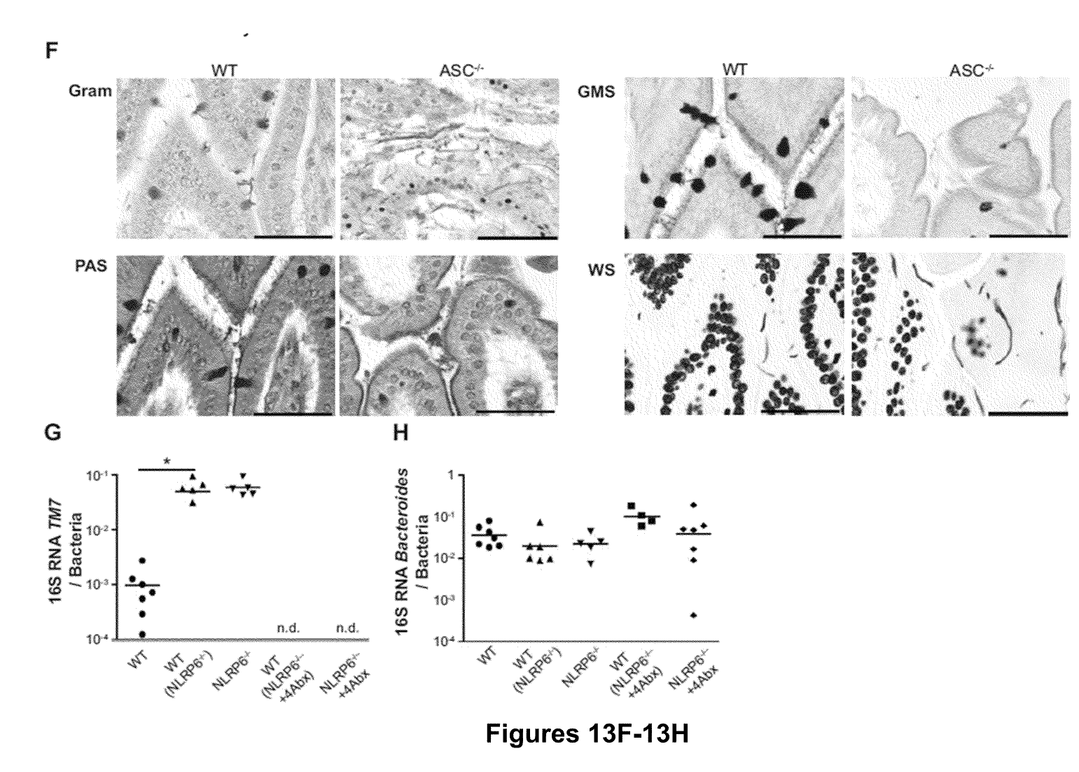

To identify whether increased colitis severity is driven by bacterial components, ASC^{-/-} mice were first treated with a combination of antibiotics known to reduce the proportional representation of a broad range of bacterial phylotypes in the gut (Suzuki et al., 2004, Proc. Natl. Acad. Sci. USA 101:1981-1986; Rakoff-Nahoum et al., 2004, Cell 118:229-241). Antibiotic therapy reduced the severity of DSS colitis in ASC^{-/-} mice to WT levels (FIGS. 13A-13B). To exclude a possible role for herpes viruses, fingi, and parasites, single-housed WT and ASC^{-/-} mice were treated for 3 weeks with oral gancyclovir, amphotericin, or albendazole and praziquantel, respectively. None of these treatments altered the severity of colitis in ASC-deficient mice (FIGS. 13C-13E). Furthermore, fecal tests for rotavirus, lymphocytic choriomeningitis virus, K87, murine cytomegalovirus, mouse hepatitis virus, mouse parvovirus, reovirus, and Theiler's murine encephalomyelitis virus were all negative, and there was no histological evidence of inclusion bodies, which are characteristic of virally infected colonic epithelial cells. Together, these results pointed to bacterial components as being responsible for the transferrable colitis phenotype in NLRP6 inflammasome-deficient mice.

FIG. 35 lists bacterial phylotypes whose presence or absence was significantly different in (i) single-housed WT mice compared to (ii) ASC^{-/-} and NLRP6^{-/-}, and caspase-1^{-/-}, and IL-18^{-/-}, and all types of cohoused WT mice (all untreated with DSS). Nine genera belonging to four phyla (Firmicutes, Bacteroidetes, Proteobacteria, and TM7) satisfied the requirement of having significant differences in their representation in the fecal microbiota in group (i) versus group (ii). The genus-level phylotype that is most significantly associated with the fecal microbiota of ASC^{-/-}, NLRP6^{-/-}, caspase-1^{-/-}, IL-18^{-/-}, and cohoused WT mice was a member of the family Prevotellaceae in the phylum Bacteroidetes. Beyond this unnamed genus in the Prevotellaceae, the next two most discriminatory genus-level taxa belonged to the phylum TM7 and the named genus Prevotella within the Prevotellaceae (FIG. 3E and FIG. 9G). Likewise, Prevotellaceae was absent from single-housed CCL5^{-/-} mice and highly acquired following cohousing with NLRP6^{-/-} mice (FIGS. 12J-12K). Also included in this list was a member of the family Helicobacteraceae (order Campylobacterales); tests for the pathogen Helicobacter hepaticus were consistently negative in these mice (n=6 samples per strain screened with PCR).

Histopathologic analyses of colonic sections stained with hematoxylin and eosin as well as Warthin-Starry stain disclosed microbes with a long branching, striated morphotype that is closely associated with the crypt epithelium of single-housed ASC^{-/-} and NLRP6^{-/-} mice; these organisms were rare in WT mice (FIG. 13F. This morphotype is consistent with members of TM7 (Hugenholtz et al., 2001, Appl. Environ. Microbiol. 67:411-419). Quadruple antibiotic treatment for 3 weeks eliminated microbes with this morphology from ASC^{-/-} mice as judged by histopathologic analysis (n=5 mice).

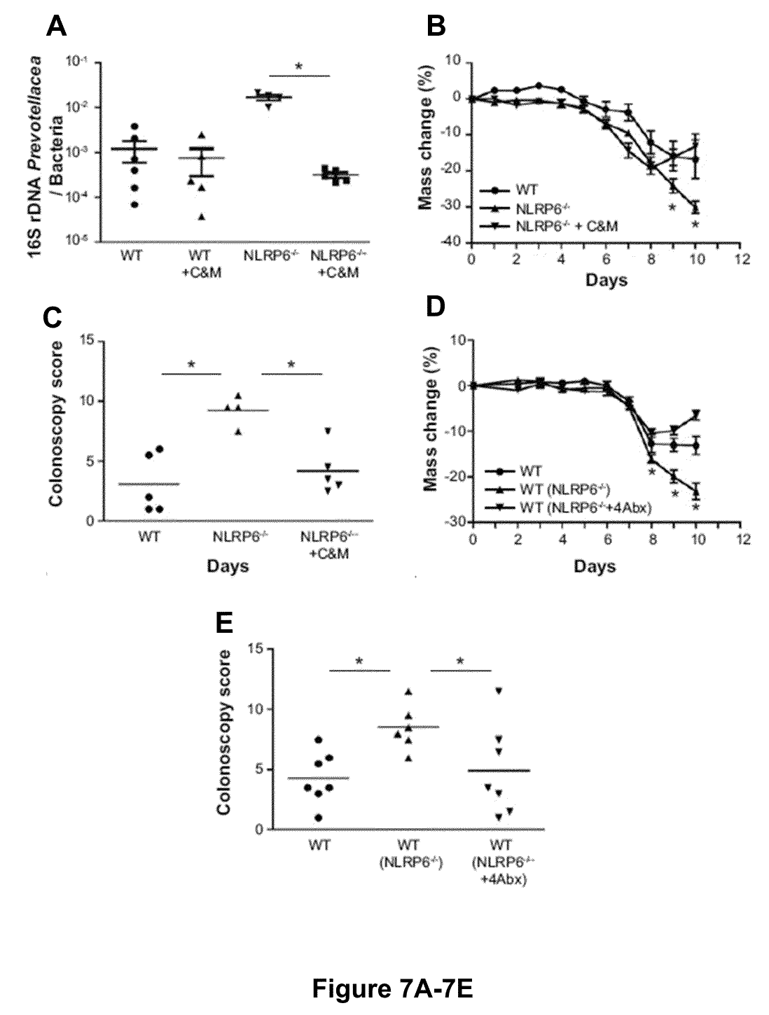

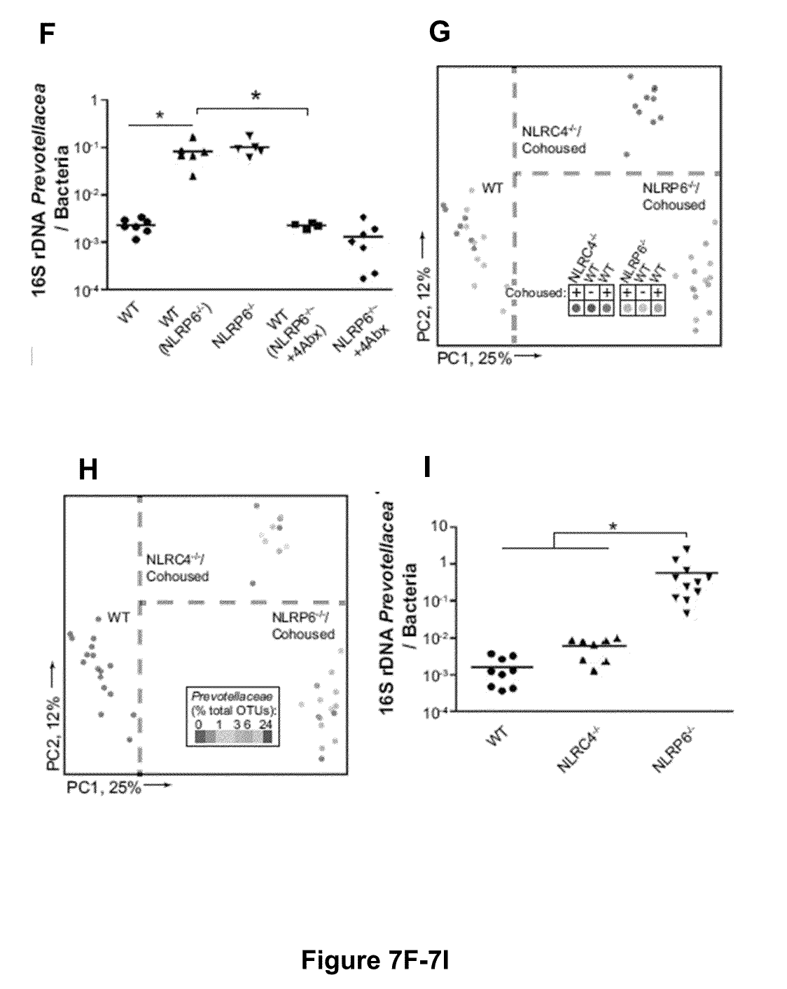

A significant reduction in Prevotellaceae was noted in stools of NLRP6^{-/-} mice that were treated with the same combination of four antibiotics. The most complete eradication was achieved using a combination of metronidazole and ciprofloxacin, a commonly used regimen for treatment of human IBD (FIG. 7A). The severity of DSS colitis was also significantly reduced in antibiotic-treated compared to untreated NLRP6^{-/-} mice (FIGS. 7B-7C).

Next, whether antibiotic treatment affected the ability of NLRP6^{-/-} mice to transfer the colitogenic microbiota to WT mice was tested. Strikingly, WT mice cohoused with antibiotic-treated NLRP6^{-/-} mice developed significantly less-severe DSS colitis compared to WT mice cohoused with untreated NLRP6^{-/-} mice (FIGS. 7D-7E). This reduction in severity correlated with decreased abundance of Prevotellaceae and TM7, but not of Bacteroidetes in WT mice cohoused with antibiotic-treated NLRP6^{-/-} mice (FIG. 7F and FIGS. 13G-13H). Low-level representation of Prevotellaceae was noted in nonphenotypic NLR-deficient mice bred for generations in our laboratory's vivarium (FIG. 35). As representative NLRs, the quantitative differences in Prevotellaceae abundance and its impact on transmissibility to WT mice between NLRP6^{-/-} and NLRC4^{-/-} mice were directly compared, as the latter lacks a closely related colonic-epithelium-expressed protein that is also able to form an inflammasome and process IL-18. Indeed, NLRC4^{-/-} and their cohoused WT cagemates featured a clustering pattern in the PCoA plot (FIG. 7G) distinct from both single-housed WT mice as well as from NLRP6^{-/-} mice and cohoused WT mice. Specifically, Prevotellaceae was highly abundant in NLRP6^{-/-} mice though low to absent in NLRC4^{-/-} mice, their cohoused WT cagemates, and single-housed WT mice (FIG. 7H).

To determine whether NLRP6 deficiency was associated with an alteration in the physical distribution (biogeography) of the microbiota within the gut, colon tissue that had been thoroughly washed of fecal matter was analyzed (as described above). This enabled enhanced detection of bacteria residing in crypts. TM7 and Prevotellaceae were significantly more prevalent in the washed colons of NLRP6^{-/-} mice compared to WT and NLRC4^{-/-} mice (FIG. 7I). Further, transmission electron microscopy studies revealed multiple monomorphic bacteria in crypt bases of ASC^{-/-} and NLRP6^{-/-}, but not WT and NLRPC4^{-/-} mice, featuring an abundance of electron dense intracellular material that was consistent with the pigmentation that is characteristic of many Prevotella species (FIGS. 7J-7L). Overall, these findings are consistent with the explanation that the dysbiosis in NLRP6 inflammasome-deficient mice may involve aberrant host-microbial cross-talk within the colonic crypt.

### Example 2

### Inflammasome-Mediated Dysbiosis Regulates Progression of NAFLD and Obesity

The results described herein provide evidence that modulation of the intestinal microbiota through multiple inflammasome components is a critical determinant of NAFLD/NASH progression as well as multiple other aspects of metabolic syndrome such as weight gain and glucose homeostasis. These results demonstrated a complex and cooperative effect of two sensing protein families, namely NLRs and TLRs, in shaping metabolic events. In the gut, the combination of host-related factors such as genetic inflammasome deficiency-associated dysbiosis resulted in abnormal accumulation of bacterial products in the portal circulation. The liver, being a ‘first pass’ organ and thus exposed to the highest concentration of portal system products such as PAMPs, was expected to be most vulnerable to their effects, particularly when pre-conditioned by sub-clinical pathology such as lipid accumulation in hepatocytes. Indeed in these models, accumulation of TLR agonists was sufficient to drive progression of NAFLD/NASH even in genetically intact animals.

This ‘gut-liver axis’, driven by alterations in gut microbial ecology, may offer an explanation for a number of long-standing, albeit poorly understood, clinical associations. One example is the occurrence of primary sclerosing cholangitis (PSC) in patients with inflammatory bowel disease, particularly those with inflammation along the length of the colon. Coeliac disease, another inflammatory disorder with increased intestinal permeability, is associated with a variety of liver disorders, ranging from asymptomatic transaminasaemia, NAFLD, to primary biliary cirrhosis (PBC). In fully developed cirrhosis, complications associated with high mortality such as portal hypertension, variceal bleeding, spontaneous bacterial peritonitis and encephalopathy are triggered by translocation of bacteria or bacterial components, providing another important example of the importance of the interplay between the microbiome, the immune response and liver pathology (Almeida et al., 2006, World J. Gastroenterol. 12:1493-1502)

Recent reports suggest a complex role of inflammasome function in multiple manifestations of the metabolic syndrome. In agreement with previous studies, we found increased obesity and insulin resistance in IL18-deficient mice fed with a HFD. However, and in contrast to two previous reports (Wen et al, 2011, Nature Immunol. 12:408-415; Stienstra et al., 2011, Proc. Nat. Acad. Sci. USA 108:15324-15329), it is herein shown that Asc^{-/-} mice are prone to obesity induction and hepatosteatosis, as well as impaired glucose homeostasis when fed a HFD. Alterations in intestinal microbiota communities associated with multiple inflammasome deficiencies could account for these discrepancies and it should be added to the list of major environmental/host factors affecting manifestations and progression of metabolic syndrome in susceptible populations.

In the inflammasome-deficient setting, a significant expansion of Porphyromonadaceae was found following administration of MCDD and HFD, which was abolished by antibiotic treatment. Interestingly, one member of the family, Porphyromonas, has been associated with several components of the metabolic syndrome in both mice and humans, including atherosclerosis and diabetes mellitus (Bajaj et al., 2011, Am. J. Physiol. Gastrointest. Liver Physoil. 302:168-175; Makiura et al., 2008, Oral Microbiol. Immunol. 23:348-351). Moreover, expansion of this taxa is strongly associated with complications of chronic liver disease (Bajaj et al., 2011, Am. J. Physiol. Gastrointest. Liver Physoil. 302:168-175).

The materials and methods employed in these experiments are now described.

### Mice

Casp1^{-/-} (Casp1^{tm1Flv}) and Nlrp4c^{-/-} mice were generated (Sutterwala et al., 2006, Immunity 24:317-327). Production of ASC^{-/-} (Pycard^{tm1Flv)}, Nlrp3^{-/-}, NIrp6^{-/-}, Nrc4^{-/-} and Nlrp12^{-/-} mice is described elsewhere (Elinav et al., 2011, Cell 145:745-757). IL18^{-/-} (IL18^{tm1Aki}), ILr^{-/-} (IL1r1^{tm1Imx}), Tnf^{-/-} (Tnf^{tm1Gkl}), Tlr4^{-/-} (Tlr4^{lps-del}), Tlr5^{-/-} (Tlr5^{tm1Flv}), Myd88^{-/-} (Myd88^{tm1Defr}), Ccl5^{-/-} (Ccl5^{tm1Hso}), Rag1^{-/-} (Rag1^{tm1Mom}), CD11c-Cre (Itgax-cre), albumin-Cre (Alb-cre), Trif^{-/-} (Ticam1^{Lps2}) and db/db(Lepr^{db}) mice were obtained from Jackson Laboratories (Bar Harbor, Me.). Tlr9^{-/-} mice have been described in another report (Hemmi et al., 2000, Nature 408:740-745). Production of Nlrp3KI (A350V) mice is described elsewhere (Brydges et al., 2009, Immunity 30:875-887). Wild-type C57Bl/6 mice were purchased from the NCl. For co-housing experiments, age-matched wild-type and KO mice at the age of 4-6 weeks were co-housed in sterilized cages for 4 or 12 weeks at a ratio of 1:1 (WT:KO), with unrestricted access to food and water. No more than 6 mice in total were housed per cage. For antibiotic treatment, mice were given a combination of ciprofloxacin (0.2 g l⁻¹) and metronidazole (1 g l⁻¹) for 4 weeks in the drinking water. All antibiotics were obtained from Sigma Aldrich (St. Louis, Mo.). All experimental procedures were approved by the local IACUC.

### NASH Model

6-8-old male mice were fed a methionine-choline-deficient diet (MP Biomedicals) for 24 days. Methionine-choline-sufficient control diet was the same but supplemented with choline chloride (2 g per kg of diet) and di-methionine (3 g per kg of diet). Mice had unrestricted access to food and water.

### High Fat Diet Model

8-10 week-old male mice were fed a HFD ad libitum. This diet consists of 60% calories from fat (D 12492i; Research Diets) and was administered for 10-12 weeks.

### Histology

The intact liver was excised immediately after mice were euthanized by asphyxiation, fixed in 10% neutral buffered formalin and embedded in paraffin. Liver sections were stained with haematoxylin and eosin, or trichrome. Histological examination was performed in a blinded fashion by an experienced gastrointestinal pathologist with the histological scoring system for NAFLD (Kleiner et al., 2005, Hepatology 41:1313-1321). Briefly, steatosis and inflammation scores ranged from 0 to 3 with 0 being within normal limits and 3 being most severe. Individual scores were assigned for each parameter. The most severe area of hepatic inflammation of representative histology sections were photographed using an Olympus microscope.

Colons were fixed in Bouin's medium and embedded in paraffin. Blocks were serially sectioned along the cephalocaudal axis of the gut to the level of the lumen; 5-μm-thick sections were stained with haematoxylin and eosin. Digital light microscopic images were recorded with a Zeiss Axio Imager.A1 microscope, AxioCam MRc5 camera and AxioVision 4.7.1 imaging software (Carl Zeiss Microimaging) (Elinav et al., 2011, Cell 145:745-757).

### Gene Expression Analysis

Tissues were preserved in RNAlater solution (Ambion), and subsequently homogenized in TRIzol reagent (Invitrogen, Grand Island, N.Y.). RNA (1 μg) was used to generate complementary DNA using the HighCapacity cDNA Reverse Transcription kit (Applied Biosystems, Carlsbad, Calif.). Real time PCR was performed using gene-specific primer/probe sets (Applied Biosystems, Carlsbad, Calif.) and Kapa Probe Fast qPCR kit (Kapa Biosystems, Woburn, Mass.) on a 7500 Fast Real Time PCR instrument (Applied Biosystems, Carlsbad, Calif.). The reaction conditions were 95° C. for 20 seconds, followed by 40 cycles of 95° C. for 3 seconds and 60° C. for 30 seconds. Data was analysed using the Sequence Detection Software according to the ΔCₜ method with Hprt serving as the reference housekeeping gene.

### Glucose Tolerance Test (GTT)

GTTs were performed after 10-12 weeks of consuming the HFD. Mice were fasted overnight (˜14 h), and injected intraperitoneally with 10% dextrose at a dose of 1 g per kg body weight. Blood was collected from tail vein and plasma glucose levels measured at indicated times using a YSI 2700 Select Glucose Analyzer (YSI Life Sciences, Yellow Springs, Ohio). Plasma insulin levels were determined by radioimmunoassay (Linco).

### Flow Cytometry Analysis

Livers were collected, digested with 0.5 mg ml⁻¹ collagenase IV (Sigma) for 45 minutes at 37° C., homogenized and repeatedly centrifuged at 400 g for minutes to enrich for haematopoietic cells. Cells were stained for flow cytometry using antibodies against CD45.2, CD11b, CD11c, NK1.1, B220, CD4, CD8, TCRβ, F4/80, Gr-1, MHC class II (Biolegend) and analysed on a BD LDR II.

### Portal Vein Blood Collection

Mice were anaesthetized with ketamine 100 mg per kg and xylazine 10 mg per kg, Mice were placed on a clean surgical field, and the abdominal fur was clipped and cleaned with a two stage surgical scrub consisting of Betadine and 70% ethanol. A 1 to 1.5 cm midline incision was made in the skin and abdominal wall. The peritoneum was moved to the left and the portal vein was punctured with a 30G needle. Between 0.2 and 0.3 ml of blood were collected per mouse. Serum was recovered by centrifugation at 1,500 g for 15 minutes at room temperature and then stored at −80° C. in endotoxin-free tubes until assayed.

### Measurement of PAMPs

TLR2, TLR4 and TLR9 agonists were assayed in portal vein serum using HEK-blue mTLR2, HEK-blue mTLR4 and HEK-blue mTLR9 reporter cell lines (InvivoGen, San Diego, Calif.) and the manufacturer's protocol with modifications. In brief, 2.2×10⁵ HEK-blue mTLR2, 1.0×10⁵ HEK-blue mTLR4 and 2.0×10⁵ HEK-blue mTLR9 cells were plated in 96-well plates containing 10 μl of heat-inactivated (45 minutes at 56° C.) portal vein serum. Cells were then incubated for 21 hours at 37° C. under an atmosphere of 5% CO₂/95% air. Twenty microlitres of the cell culture supernatants were collected and added to 180 μl of the QUANTI-Blue substrate in a 96-well plate. The mixtures were then incubated at 37° C. in 5% CO₂/95% air for 3 hours and secreted embryonic alkaline phosphatase levels were determined using a spectrophotometer at 655 nrm.

### Transmission Electron Microscopy

Mice were perfused via their left ventricles using 4% paraformaldehyde in PBS. Selected tissues were fixed in 2.5% glutaraldehyde in 0.1 M sodium cacodylate buffer pH 7.4 for 1-2 h. Samples were rinsed three times in sodium cacodylate buffer and post-fixed in 1% osmium tetroxide for 1 hour, en bloc stained in 2% uranyl acetate in maleate buffer pH 5.2 for a further hour then rinsed, dehydrated, infiltrated with Epon812 resin, and baked overnight at 60° C. Hardened blocks were cut using a Leica UltraCut UCT. 60-nm-thick sections were collected and stained using 2% uranyl acetate and lead citrate. Samples were all viewed in an FEI Tencai Biotwin TEM at 80 kV. Images were taken using Morada CCD and iTEM (Olympus) software (Elinav et al., 2011, Cell 145:745-757).

### Bone Marrow Chimeras

Bone marrow was flushed from femurs with DMEM with 10% FBS, red cells were lysed, and the material filtered through a 70 μm filter. 10⁶ cells in 100 μl PBS were delivered by retro-orbital injection into lethally irradiated (1,000 rad) mice. For 2 weeks post-engraftment, mice were maintained on antibiotics (Sulfatrim). Six weeks after transplantation animals were switched to MCDD. A wild-type non-irradiated mouse was co-housed with the engrafted mice for 4 weeks before NASH induction. Under this protocol, bone marrow chimaeras routinely show a level of engraftment of ≧93%.

Bacterial 16S rRNA Amplicon Sequencing

Total DNA was isolated from the livers of mice fed a MCDD diet and used for attempted PCR amplification of variable region 2 of bacterial 16S rRNA genes (Seki et al., 2007, Nature Med. 13:1324-1332) that may be present in the tissue. Thirty cycles of amplification of liver DNA prepared from seven wild-type, and seven Asc^{-/-} mice yielded detectable product (>60 ng per reaction) in three samples from the wild-type group and three samples from the Asct^{-/-} group. All amplicons were then subjected to multiplex pyrosequencing with a 454 instrument using FLX Titanium chemistry (137-1,510 reads per sample, average read length, 360 nucleotides). Reads were analysed using the QIIME software package. Operational taxonomic unit (OTU) picking was performed using uclust and taxonomic assignments made with RDP (Caporaso et al., 2010, Nature Methods 7:335-336).

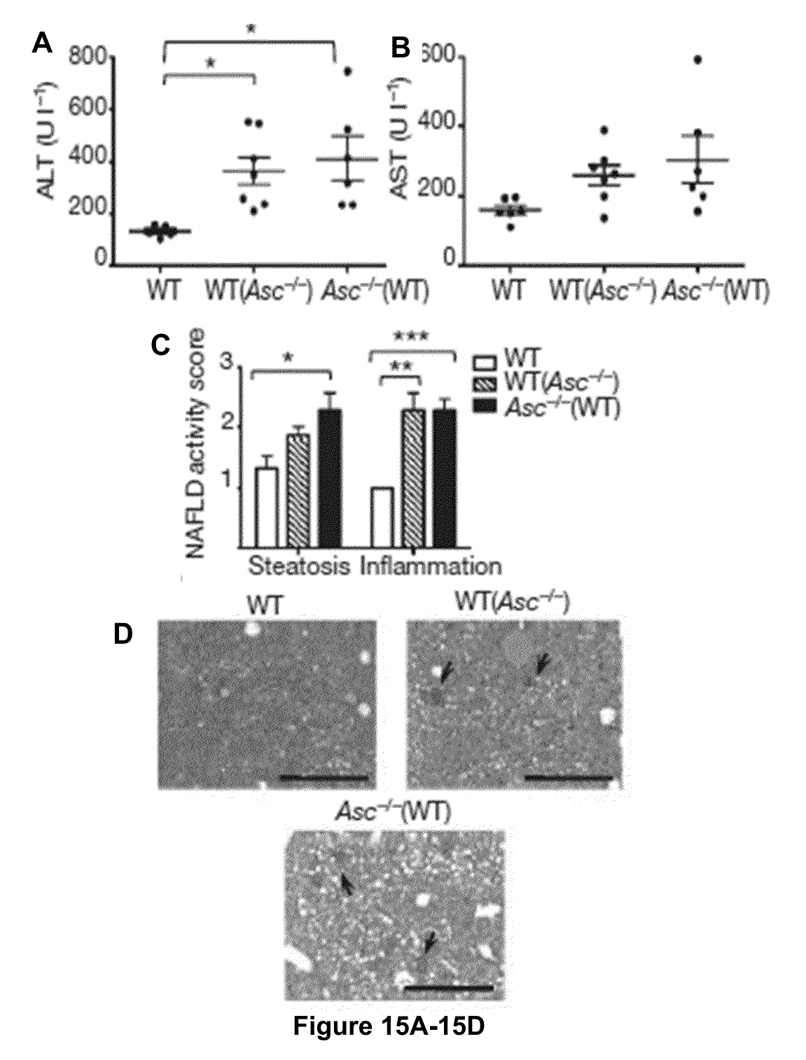

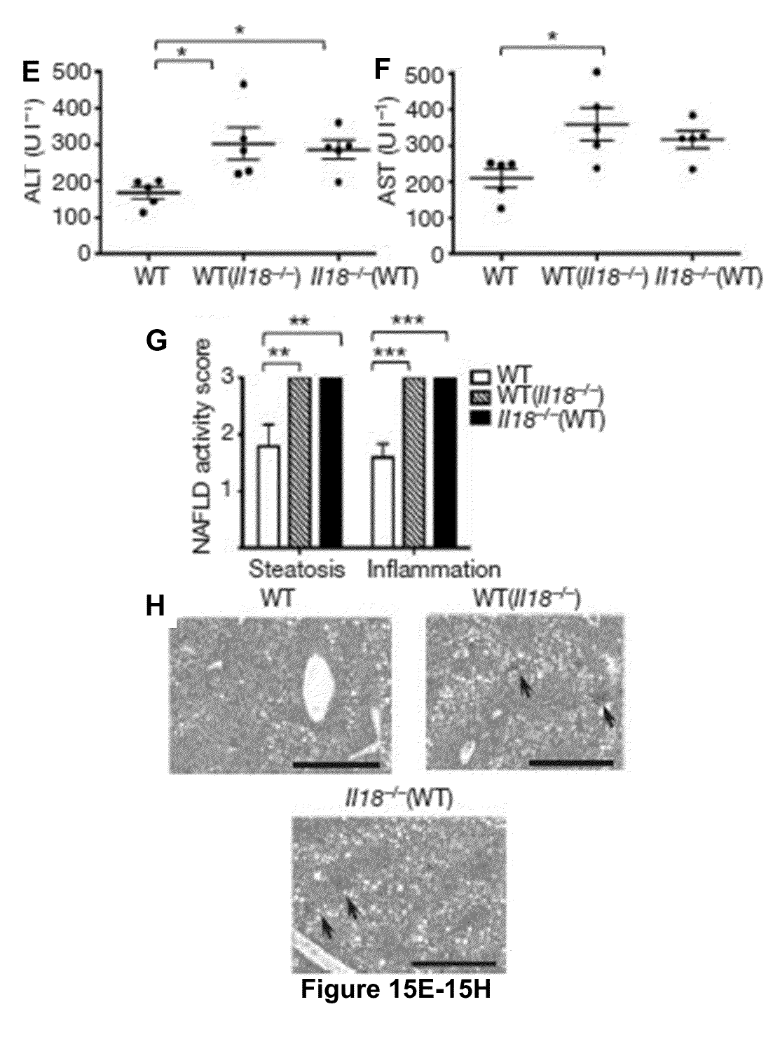

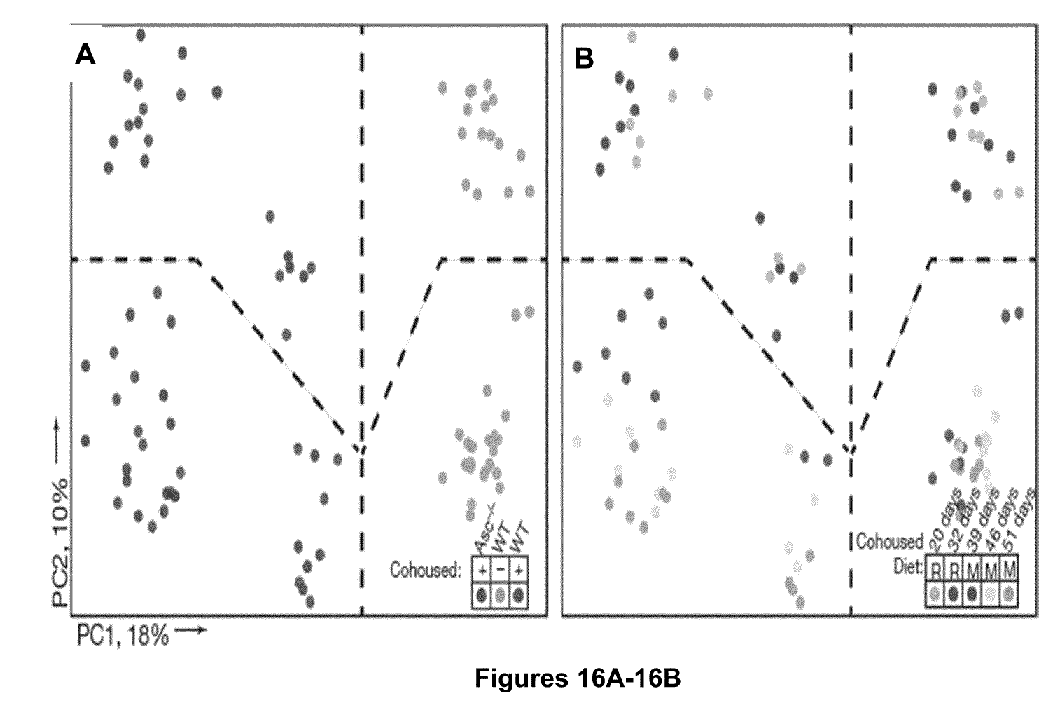

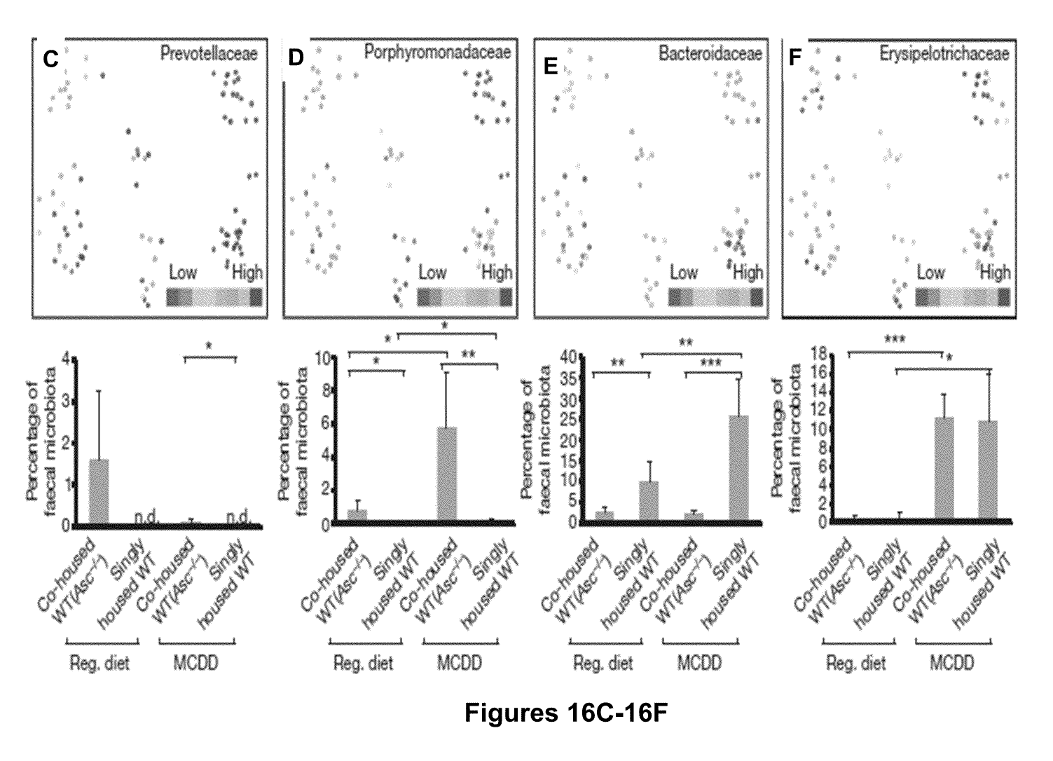

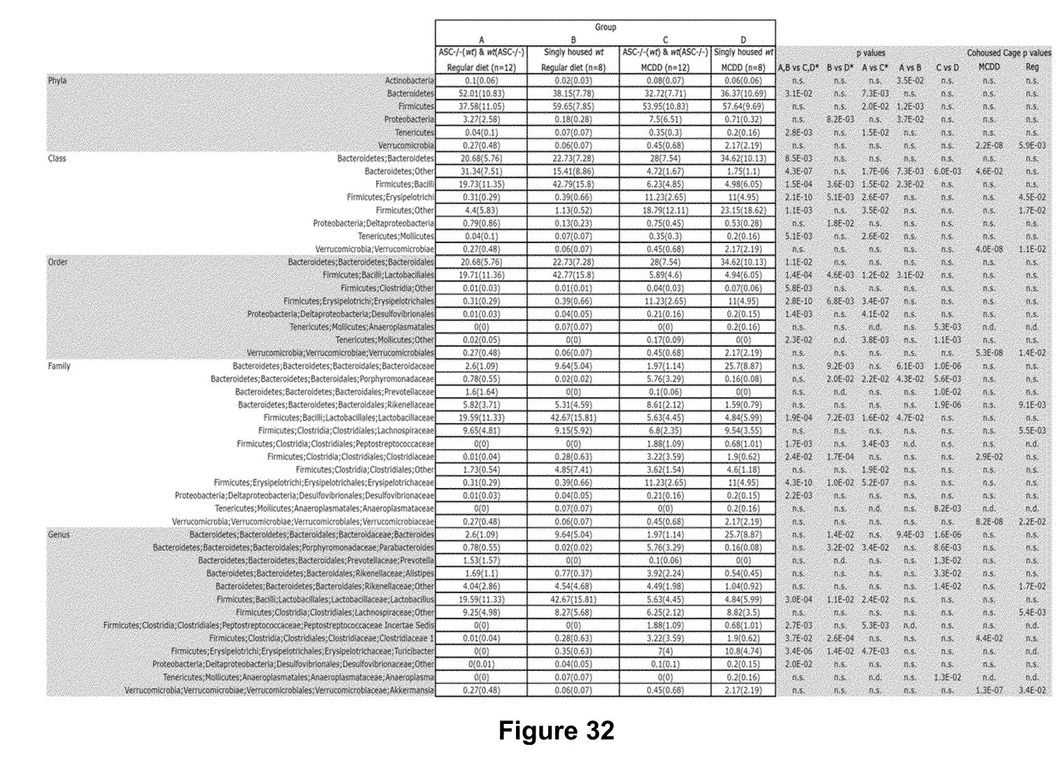

For analysis of the faecal microbiota of MCDD-fed Asc^{-/-}(WT), WT(Asc^{-/-}) and singly housed wild-type mice, faecal pellets were collected at the time points indicated in FIG. 16. The protocols that were used to extract faecal DNA and to perform multiplex pyrosequencing of amplicons generated by PCR from the V2 regions of bacterial 16S rRNA genes, have been previously described (Seki et al., 2007, Nature Med., 1324-1332). A total of 366,283 sequences were generated from 181 faecal samples (average 2,023±685 reads per sample; average read length, 360 nucleotides). Sequences were de-multiplexed and binned into species-level operational taxonomic units (OTUs; 97% nucleotide sequence identity; % ID) using QIIME 1.2.1 (Caporaso et al., 2010, Nature Methods 7:335-336). Taxonomy was assigned within QIIME using RDP. Chimaeric sequences were removed using ChimeraSlayer and OTUs were filtered to a minimum of 10 sequences per OTU and 1,000 OTUs per sample. PCoA plots were generated by averaging the unweighted UniFrac distances of 100 subsampled OTU tables. Statistical analysis was performed on the proportional representation of taxa (summarized to Phyla, Class, Order, Family and Genus levels), using paired (where possible) and unpaired t-tests. Taxa that were significantly different after multiple hypothesis testing were included in FIGS. 32-34.

### Statistical Analysis

Data are expressed as mean±s.e.m. Differences were analysed by Student's t-test or ANOVA and post hoc analysis for multiple group comparison. P values≦0.05 were considered significant.

The results of the experiments are now described.

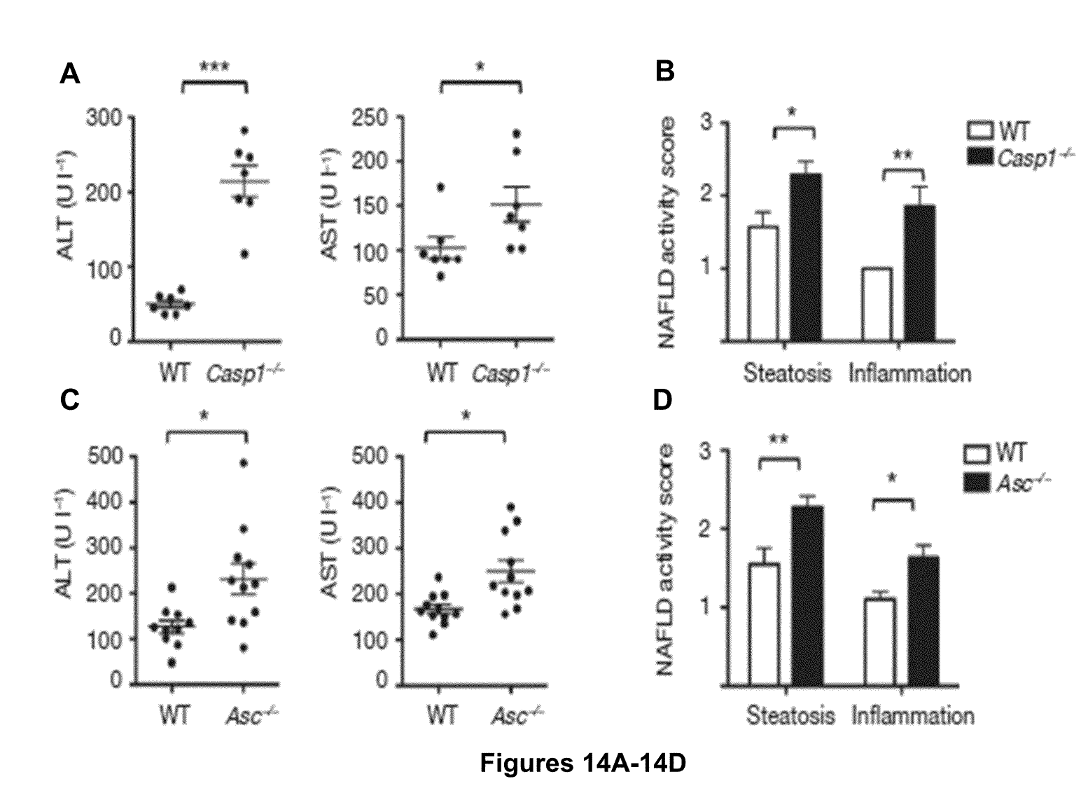

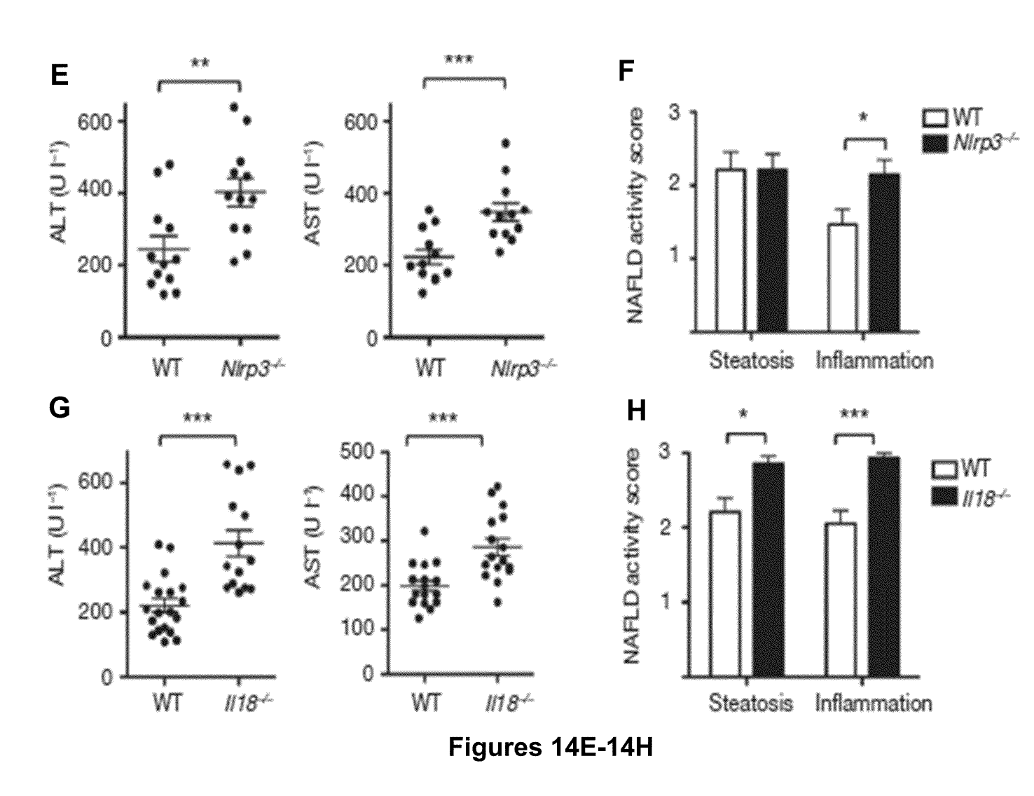

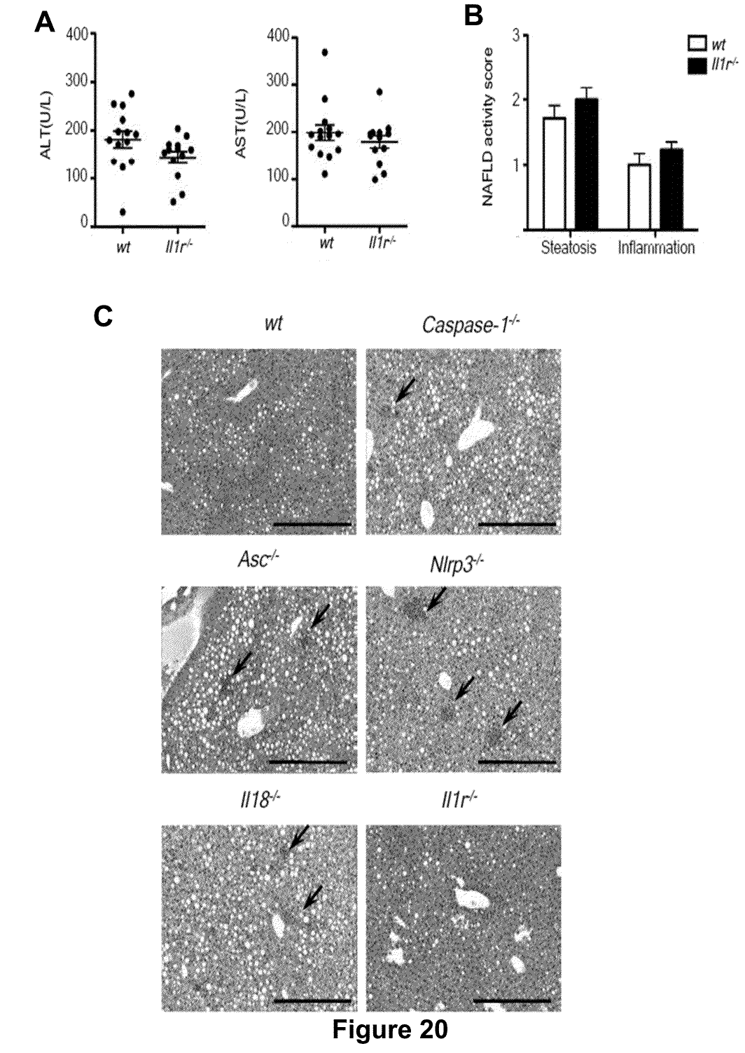

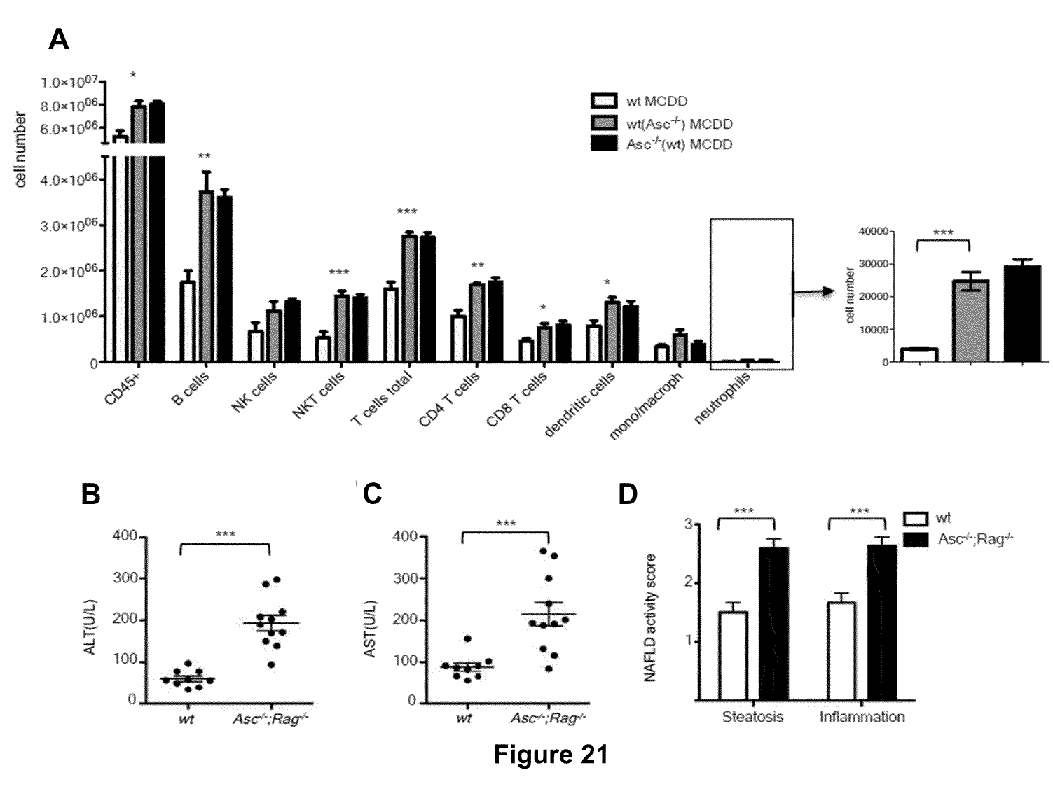

Feeding adult mice a methionine-choline-deficient diet (MCDD) for 4 weeks beginning at 8 weeks of age induces several features of human NASH, including hepatic steatosis, inflammatory cell infiltration and ultimately fibrosis (Varela-Rey et al., 2009, Int. J. Biochem. Cell Biol. 41:969-976). To investigate the role of inflammasomes in NASH progression, MCDD was fed to C57Bl/6 wild type (NCl), apoptosis-associated speck-like protein containing a CARD (Asc^{-/-}, also known as Pycard) and caspase 1 (Casp1^{-/-}) mutant mice to induce early liver damage in the absence of fibrosis (FIGS. 14A-14D and FIG. 20C). Compared to wild-type animals, age- and gender-matched Asc^{-/-} and Casp1^{-/-} mice that were fed MCDD were characterized by significantly higher serum alanine aminotransferase (ALT) and aspartate aminotransferase (AST) activity, by enhanced microvesicular and macrovesicular hepatic steatosis, and by accumulation of multiple immune subsets in the liver from the innate and adaptive arms of the immune system (as defined by pathological examination and flow cytometry; n=7-11 mice per group; FIGS. 14A-14D, FIGS. 20C and 21A). Remarkably, the hepatic accumulation of T and B cells seemed to be dispensable for this phenotype because Asc^{-/-} mice lacking adaptive immune cells (Asc^{-/-}; Rag^{-/-}) also showed more severe NASH compared to wild-type animals, and comparable degrees of pathology to Asc^{-/-} animals (FIGS. 21B-21D).

To test whether the increased NASH observed in Asc- and Casp1-deficient mice was mediated by IL-1β or IL-18, similar experiments were performedusing mice deficient in either the IL-1 receptor (IL1r^{-/-}) or IL-18 (L18^{-/-}). IL1r^{-/-} mice did not show any changes in the severity of NASH when compared to wild-type mice when fed MCDD (FIGS. 20A-20B). In contrast to, but similar to Asc^{-/-} and Casp1^{-/-} mice, MCDD-fed IL18^{-/-} animals featured a significant exacerbation of NASH severity (FIGS. 14G-14H and FIG. 20C).

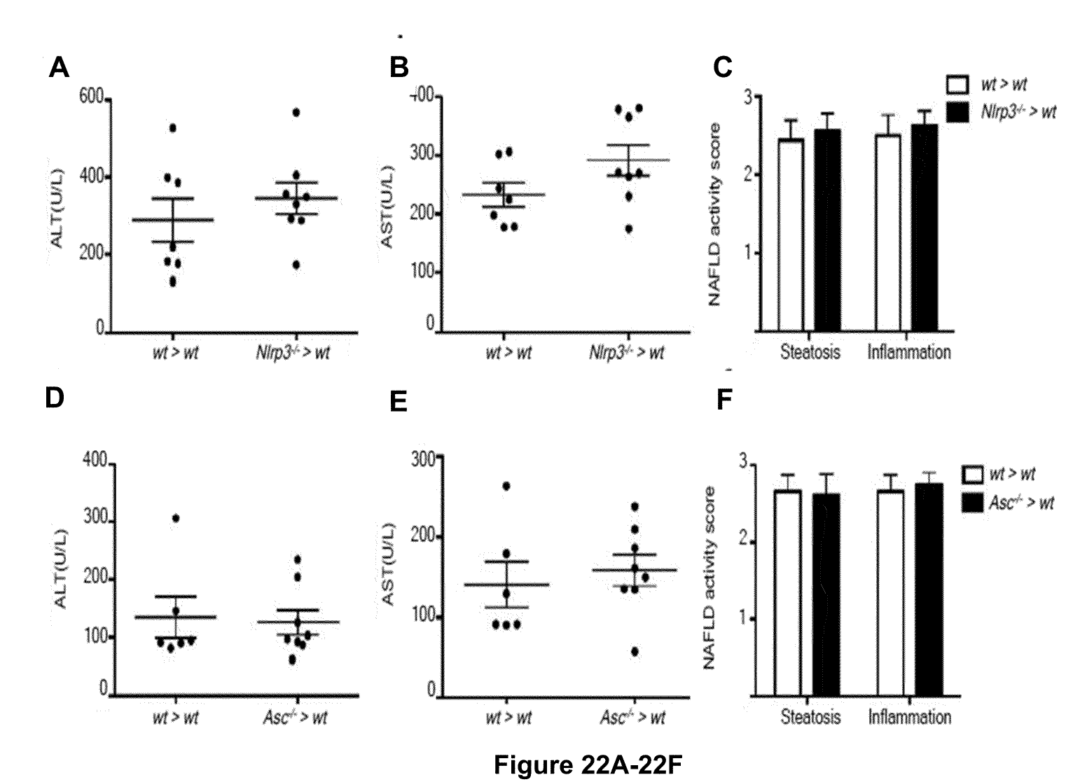

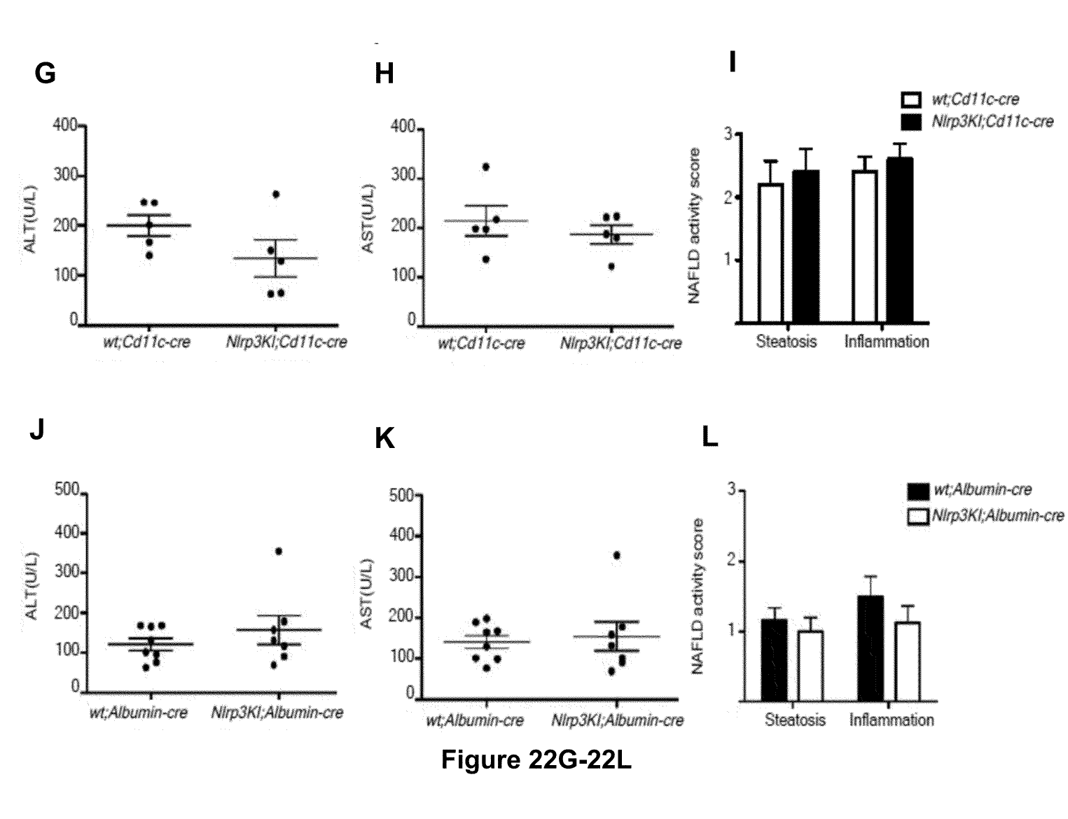

To assess the role of the NLRP3 inflammasome in NASH progression, singly housed Nlrp3^{-/-} and wild-type animals were fed MCDD for 24 days and disease progression was evaluated. Nlrp3^{-/-} mice developed exacerbated NASH compared to wild-type mice as judged by increased levels of serum ALT and AST, plus NAFLD activity inflammation scores (FIGS. 14E-14F and FIG. 20C). Remarkably, bone marrow chimaeric mice in which NLRP3 and ASC deficiency was limited to the haematopoietic compartment did not show any increase in the severity of NASH when compared to wild-type mice reconstituted with wild-type bone marrow (FIGS. 22A-22F). Likewise, knock-in mice that specifically express a constitutively active NLRP3 inflammasome in CD11c⁻ myeloid cells (Nlrp3KI; CD11c⁺-Cre) or hepatocytes (Nlrp3KI; albumin-Cre) (Brydges et al., 2009, Immunity 30:875-887) did not feature any significant differences in MCDD-induced NASH severity as compared to wild-type mice (FIGS. 22G-22L). These results indicate that aberrations in inflammasome function in cells other than hepatocytes or myeloid cells are key determinants of the enhanced disease progression in inflammasome-deficient mice.

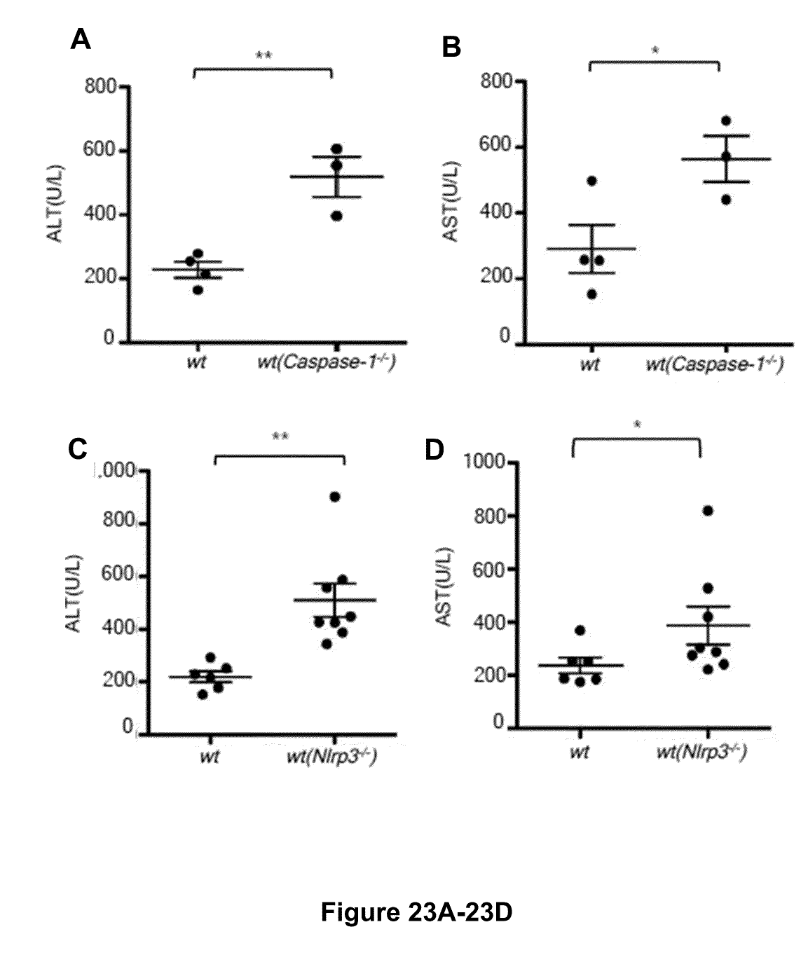

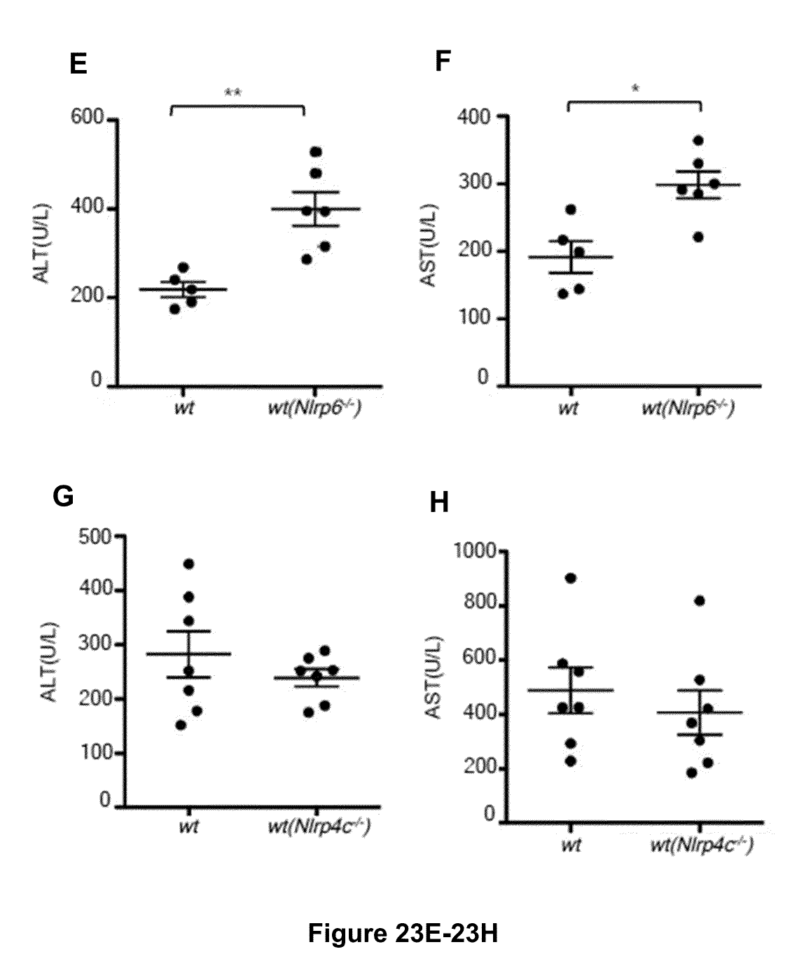

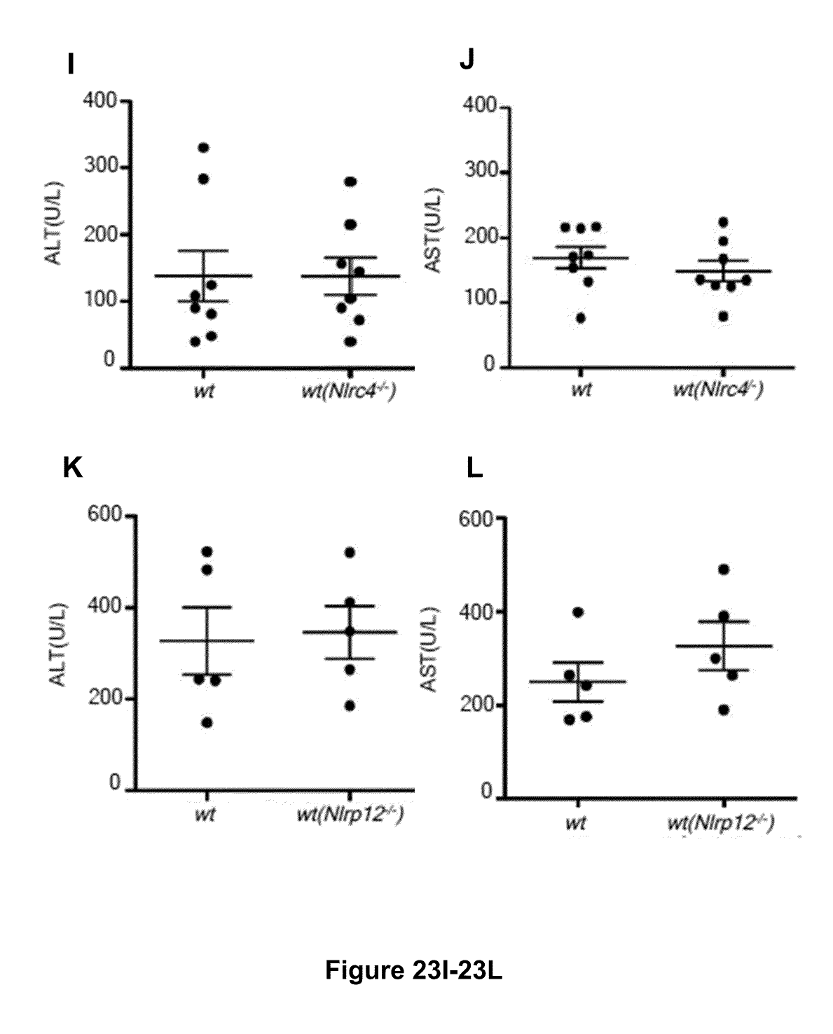

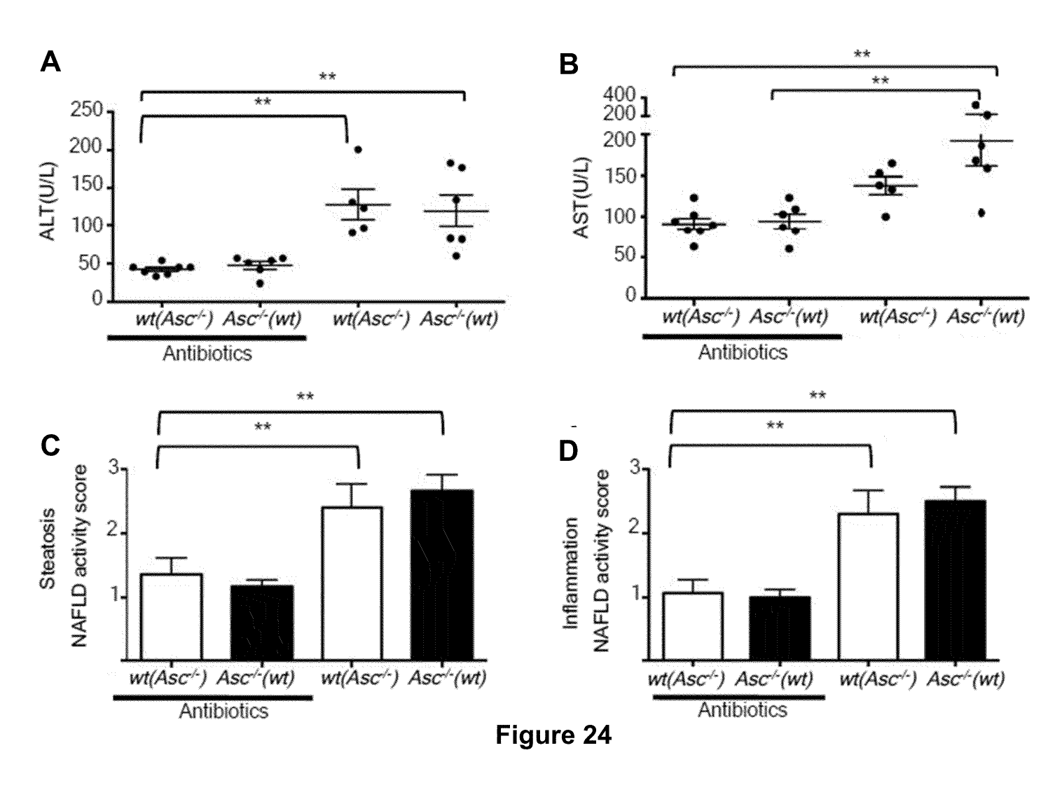

It was recently discovered that inflammasomes act as steady-state sensors and regulators of the colonic microbiota, and that a deficiency in components of two inflammasomes, NLRP6 (Elinav et al., 2011, Cell 145:745-757) and NLRP3 both of which include ASC and caspase 1, and involve IL-18 but not IL-1R, results in the development of an altered transmissible, colitogenic intestinal microbial community (Elinav et al., 2011, Cell 145:745-757). This microbiota is associated with increased representation of members of Bacteroidetes (Prevotellaceae) and the bacterial phylum TM7, and reductions in representation of members of the genus Lactobacillus in the Firmicutes phylum (Elinav et al., 2011, Cell 145:745-757). Moreover, electron microscopy studies disclosed aberrant colonization of crypts of Lieberktlhn with bacteria with morphologic features of Prevotellaceae (Elinav et al., 2011, Cell 145:745-757). Therefore, whether enhanced NASH severity in inflammasome-deficient mice is driven by their altered microbiota was investigated. Strikingly, co-housing of Asc^{-/-} and IL18^{-/-} mice with wild-type animals for 4 weeks (beginning at 4-6 weeks of age), before induction of NASH with MCDD resulted in significant exacerbation of NASH in the wild-type cage-mates (which we will refer to as WT(Asc^{-/-}) and WT(IL18^{-/-}), respectively, in the following text), as compared to singly housed, age- and gender-matched wild-type controls (n=5-7 mice per genotype per housing condition). In co-housed wild-type mice, disease severity reached comparable levels to that of co-housed Asc^{-/-} and IL18^{-/-} mice (FIGS. 15A-15H). Moreover, significantly increased numbers of multiple inflammatory cell types were present in the liver of WT(Asc^{-/-}) compared to wild-type mice (FIG. 21A). Similar findings were observed in wild-type mice co-housed with Casp1^{-/-}, Nrp3^{-/-} and Nrp6^{-/-} mice (FIGS. 23A-23F). To exclude the possibility that aberrant microbiota presented in all mice maintained in our vivarium, co-housed wild-type mice were co-housed with other strains of NLR-deficient mice that were either obtained from the same source as Asc^{-/-} and Nlrp3^{-/-} mice (Nlrc4^{-/-}, Nlrp12^{-/-}), or (Nlrp4c^{-/-}). None of these strains featured a similar phenotype (FIGS. 23G-23L). These results indicated that the transmissible colitogenic microbiota present in inflammasome-deficient mice is a major contributor to their enhanced NASH. In agreement with this, combined antibiotic treatment with ciprofloxacin and metronidazole, previously shown to abrogate the colitogenic activity of the microbiota associated with inflammasome-deficient mice associated microbiota (Elinav et al., 2011, Cell 145: 745-757), significantly reduced the severity of NASH in Asc^{-/-} mice, and abolished transmission of the phenotype to WT(Asc^{-/-}) animals (FIG. 24).

To ascertain the effects of MCDD on the gut microbiota, a culture-independent analysis of amplicons generated by primers directed against variable region 2 of bacterial 16S ribosomal RNA genes of faecal samples collected from wild-type mice co-housed with Asc^{-/-}/animals (WT(Asc^{-/-})), their Asc^{-/-} cage-mates (Asc^{-/-} (WT)) was performed as well as singly housed wild-type controls 1 day and 12 days before, and 7, 14 and 19 days after initiation of this diet (n=20 animals; 8 singly housed wild-type, 6 co-housed wild-type and 6 Asc^{-/-} mice). The structures of bacterial communities were compared based on their phylogenetic content using unweighted UniFrac. The results are illustrated in FIG. 16. FIG. 32 provides a list of all phylotypes that, based on criteria outlined in methods, discriminate co-housed WT(Asc^{-/-}) from their singly housed wild-type counterparts. Prior to MCDD, and consistent with our previous findings (Elinav et al., 2011, Cell 145:745-757), the faecal microbiota of WT(Asc^{-/-}) mice adopted a configuration similar to Asc^{-/-} cage-mates, including the appearance of Prevotellaceae (FIG. 32 and FIGS. 16A-16C). There was also a significant increase in proportional representation of members of the family Porphyromonadaceae (primarily in the genus Parabacteroides) in WT(Asc^{-/-}) mice compared to their singly housed wild-type counterparts (FIGS. 16D-16E). The representation of Porphyromonadaceae was greatly increased in both the co-housed wild-type and Asc^{-/-} mice (but not in singly housed wild-type) when they were switched to a MCDD diet (P<0.01; t-test; FIG. 16D). A dramatic increase in the family Erysipelotrichaceae (phylum Firmicutes) also occurred with MCDD in both singly and co-housed WT animals, to a level that was >10% of the community (FIG. 16F). Although the Prevotellaceae decreased when co-housed WT(Asc^{-/-}) mice were placed on MCDD, their relative abundance remained significantly higher than in singly housed wild-type animals (FIG. 16C).

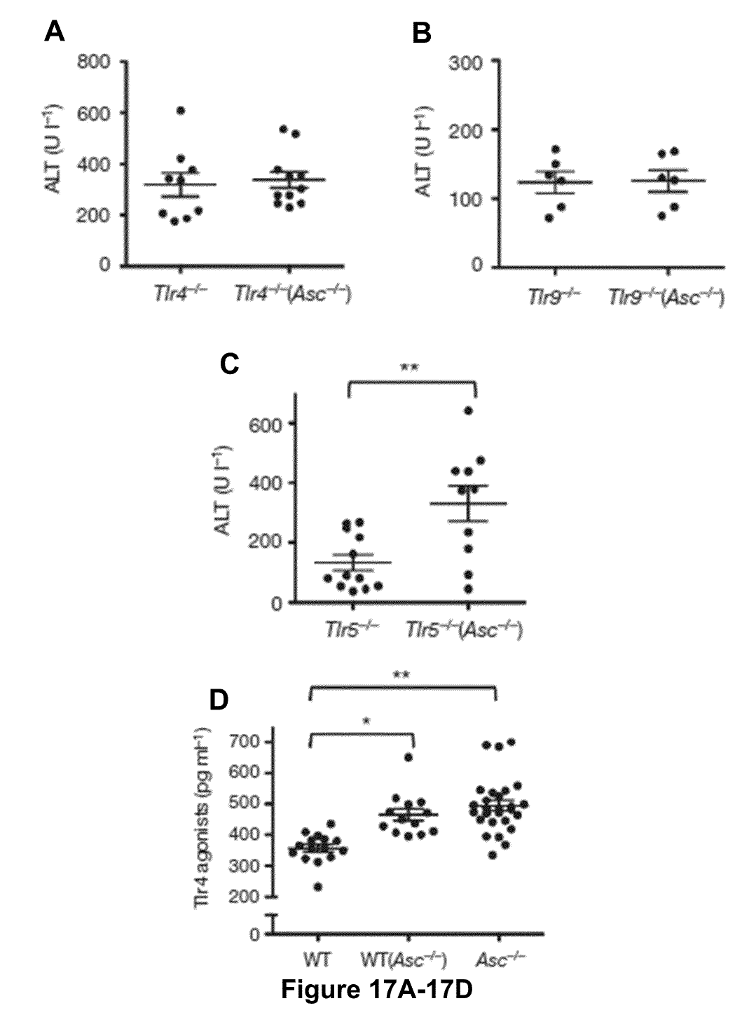

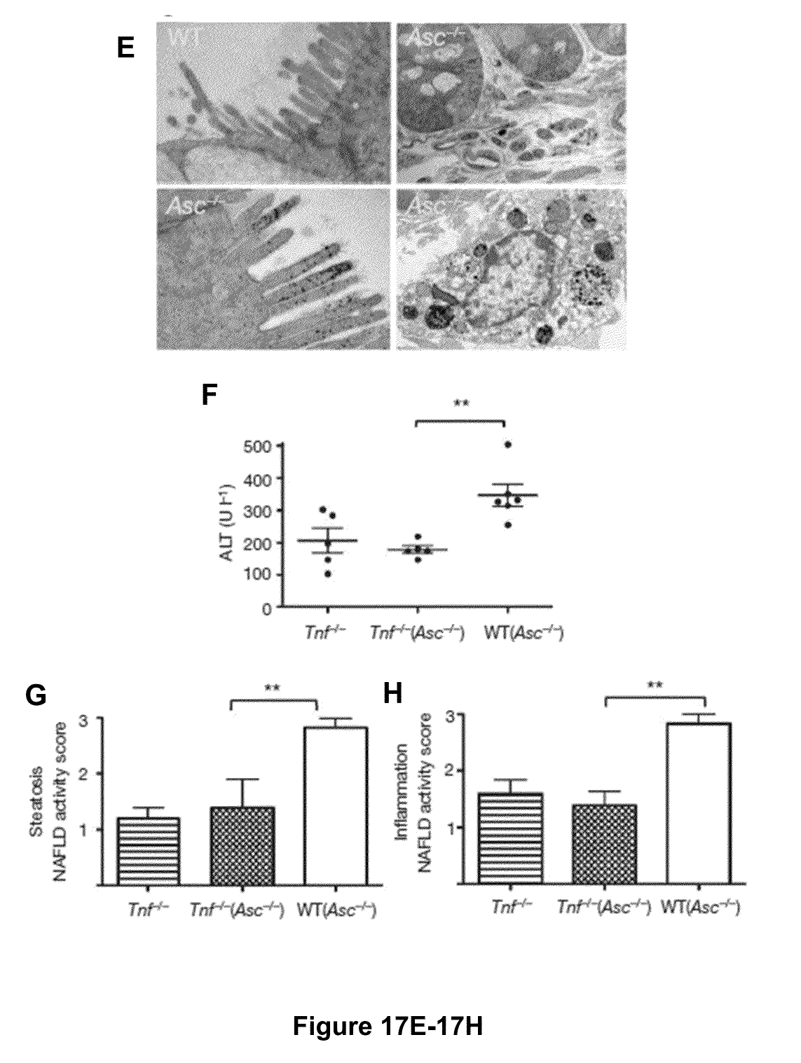

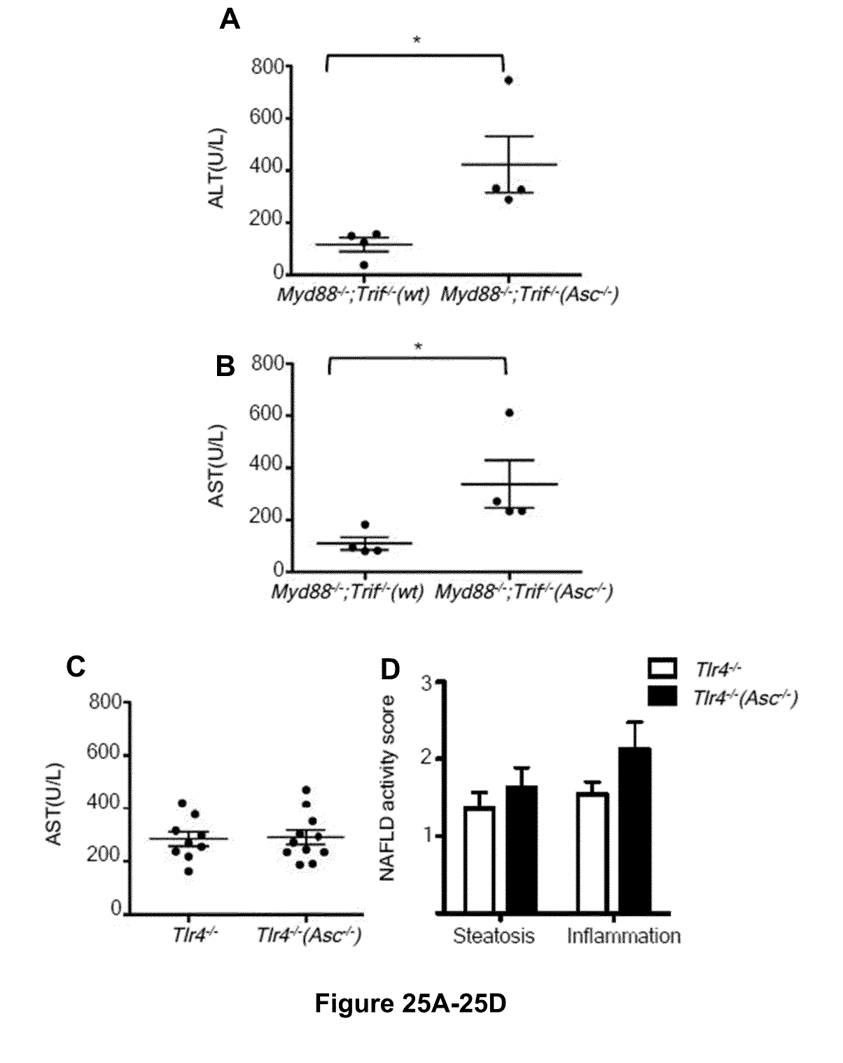

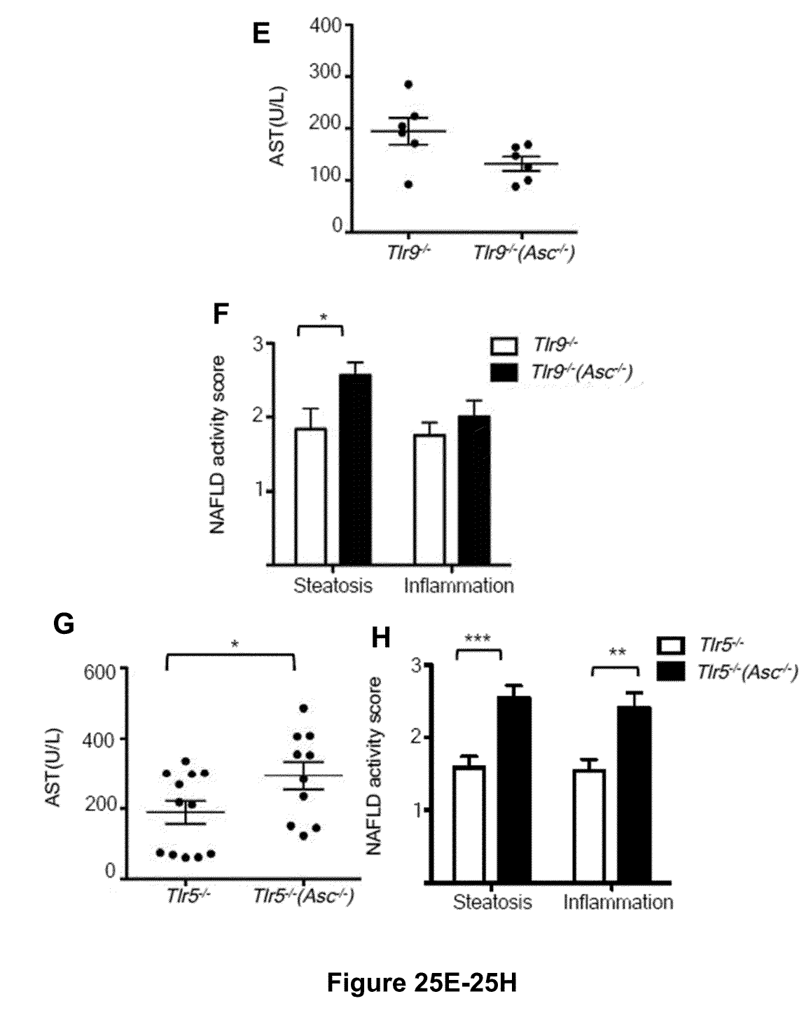

Together, these results pointed to the possibility that members of the altered intestinal microbiota in inflammasome-deficient MCDD-treated mice may promote a signaling cascade in the liver upon translocation, resulting in progression to NASH in susceptible animals. Toll-like receptors (TLR) have a major role in NAFLD pathophysiology due to the liver's exposure to relatively large amounts of PAMPs derived from the intestine and delivered via the portal circulation (Rivera et al., 2007, J. Hepatol, 47:571-579; Miura et al., 2010, Gastroenterology 139:323-334 e7; Seki et al., 2007, Nature Med. 13:1324-1332). Therefore, this is consistent with the explanation that TLR signaling mediates the increased susceptibility to progression to NASH in mice exposed to the gut microbiota of Asc^{-/-} animals. Myd88^{-/-};Trif^{-/-} mice are devoid of all TLR signaling pathways. When co-housed with Asc^{-/-} (Myd88^{-/-};Trif^{-/-}(Asc^{-/-})) mice between 5 and 9 weeks of age, they showed decreased severity of NASH after exposure to MCDD for 24 days, compared to WT(Asc^{-/-}) mice (FIGS. 25A-25B). To define which specific TLRs were responsible for the inflammatory response, Tlr4-, Tlr9- or Tlr5-deficient mice were co-housed with Asc^{-/-} animals and induced NASH with MCDD as previously described. Similar to wild-type mice, Tlr5^{-/-} mice co-housed with Asc^{-/-} mice (Tlr5^{-/-}(Asc^{-/-}) featured a statistically significant exacerbation of hepatic injury, steatosis and inflammation, when compared to singly housed Tlr5^{-/-} controls (FIG. 17C and FIGS. 25G-25H), indicating that TLR5 does not mediate the microbiota-mediated exacerbation in disease severity. In contrast, Tlr4^{-/-}(Asc^{-/-}) and Tr9^{-/-} (Asc^{-/-}) mice did not show the customary increase in disease severity when compared to their singly housed Tr4^{-/-} and Tlr9^{-/-} counterparts (FIGS. 17A-17B and FIGS. 25C-25F).

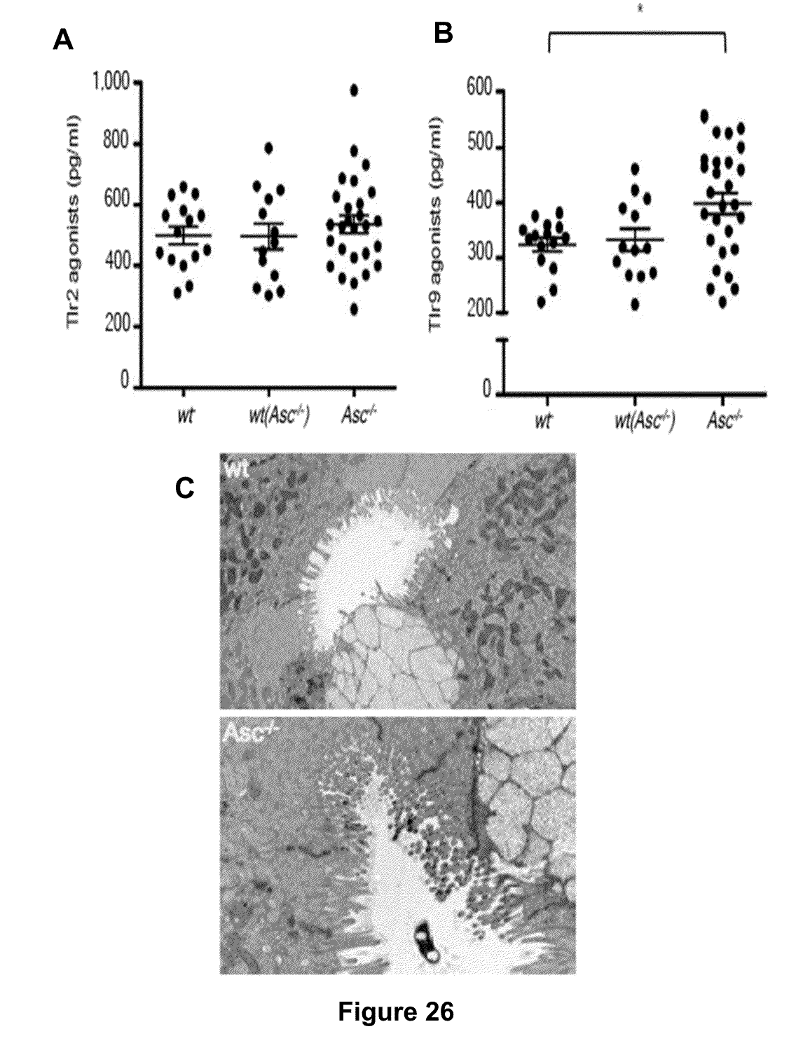

These observations indicate that intact bacteria or bacterial products derived from the intestine trigger TLR4 and TLR9 activation, which results in an increased rate of disease progression in mice that house a colitogenie gut microbiota associated with inflammasome deficiency (that is, Asc^{-/-} and WT(Asc^{-/-}) mice). Efforts to sequence 16S rRNA genes that might be present in total liver DNA, microbial quantitative PCR assays of portal vein blood DNA, histologic analysis of intact liver, and aerobic and anaerobic cultures of liver homogenates did not reveal any evidence of intact bacteria in wild-type or Asc^{-/-} mice fed MCDD, Notably, transmission electron microscopy studies of colon collected from wild-type and Asc^{-/-} mice revealed an abundance of electron-dense material, suggestive of some black-pigmented bacterial species, in colonic epithelial cells and macrophages located in the lamina propria of Asc^{-/-} mice but not in wild-type animals (FIG. 17E and FIG. 26C). In agreement with previous results, no translocations of intact bacteria were detected (FIG. 17E and FIG. 26C).

These observations provide evidence for the uptake of bacterial products from locally invasive gut microbes in Asc^{-/-} mice (FIG. 17E and FIG. 26C). If microbial components, rather than whole organisms, were transmitted to the liver then they should be detectable in the portal circulation. Indeed, levels of TLR4 and TLR9 agonists, but not TLR2 agonists (assayed by their ability to activate TLR reporter cell lines), were markedly increased in the portal circulation of MCDD-fed WT(Asc^{-/-}), and Asc^{-/-} mice compared to wild-type controls (n=13-28 mice per group; FIG. 17D and FIGS. 26A-26B). Altogether, these results indicated a mechanism whereby TLR4 and TLR9 agonist efflux from the intestines of inflammasome-deficient mice or their co-housed partners, through the portal circulation, to the liver where they trigger TLR4 and TLR9 activation that in turn results in enhanced progression of NASH.

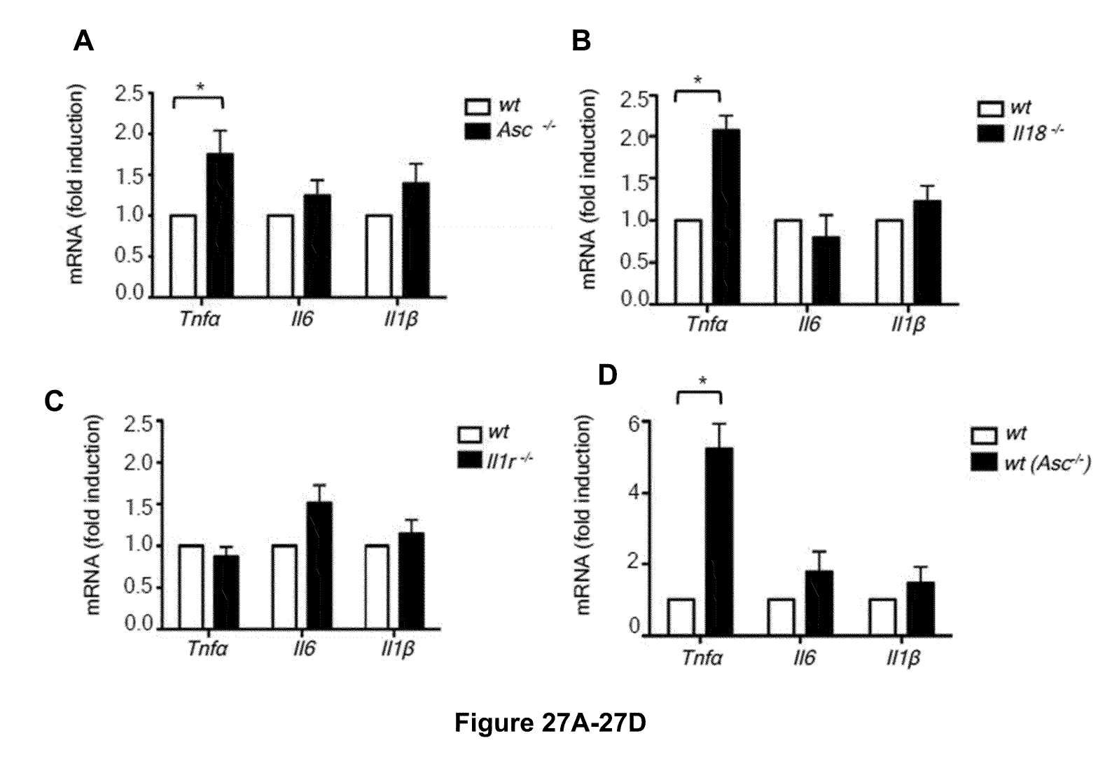

Next, the downstream mechanism whereby microbiota-induced TLR signaling enhances NASH progression was explored. Pro-inflammatory cytokines, and in particular TNF-α, a downstream cytokine of TLR signalling, are known to contribute to progression of hepatic steatosis to steatohepatitis and eventually hepatic fibrosis in a number of animal models and in human patients (Crespo et al., 2001, Hepatology 34:1158-1163; Li et al., 2003, Hepatology 37:343-350). Following induction of NASH by MCDD, hepatic Tnf mRNA expression was significantly upregulated in Asc^{-/-} and IL18^{-/-} mice, which also showed exacerbated disease. IL1r^{-/-} mice, however, did not show exacerbated disease (FIGS. 27A-27C). Moreover, Tnf mRNA levels were significantly increased in wild-type mice that had been previously co-housed with Asc^{-/-} or IL18^{-/-} mice and then fed MCDD (FIGS. 27D-27E), indicating that its enhanced expression was mediated by elements of the microbiota responsible for NASH exacerbation. In contrast, no changes in IL6 or IL1b mRNA levels in the livers of Asc^{-/-}, IL18^{-/-} or IL1r^{-/-} mice compared to wild-type controls were observed (FIGS. 27A-27C). Furthermore, whereas MCDD-administered singly housed Tnf^{-/-} mice had comparable NASH severity to singly housed wild-type animals (FIGS. 17F-17H and FIG. 27F), co-housing with Asc-deficient mice before MCDD induction of NASH resulted in increased liver injury, hepatic steatosis and inflammation in wild-type mice but not in Tnf^{-/-} mice (FIGS. 17F-17H and FIG. 27F). These results indicated that TNF-α mediates the hepatotoxic effects downstream of the transmissible gut microbiota present in Asc^{-/-} mice.

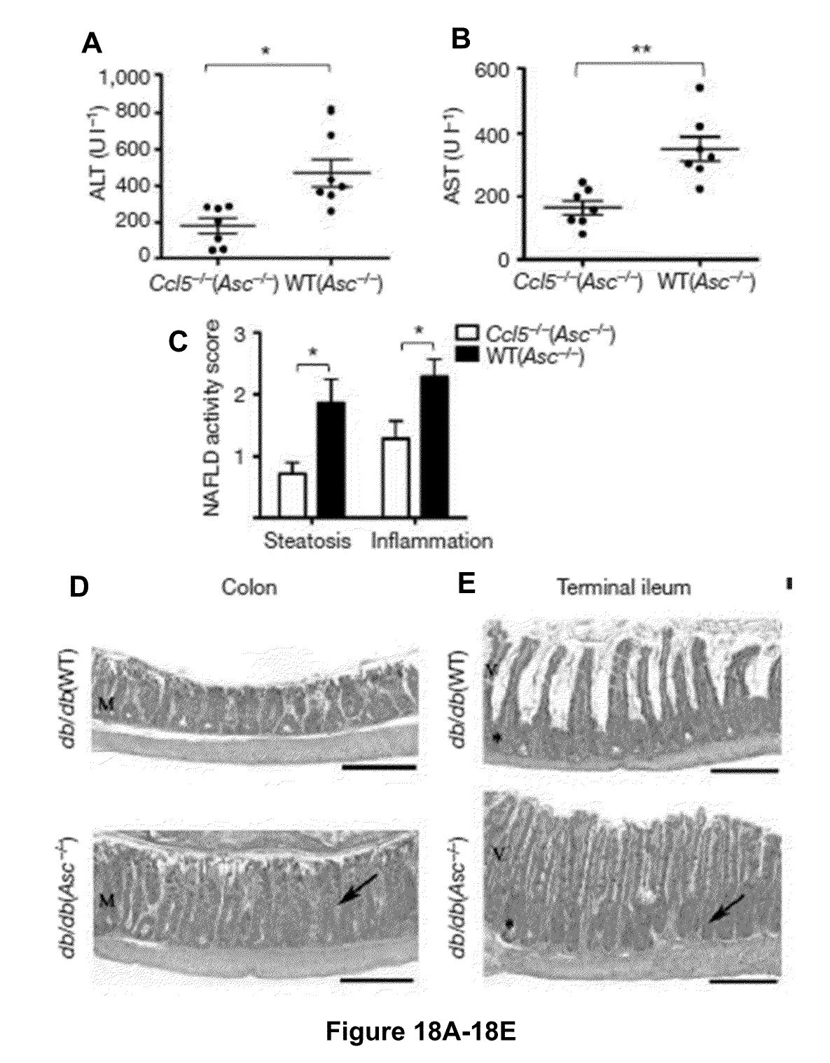

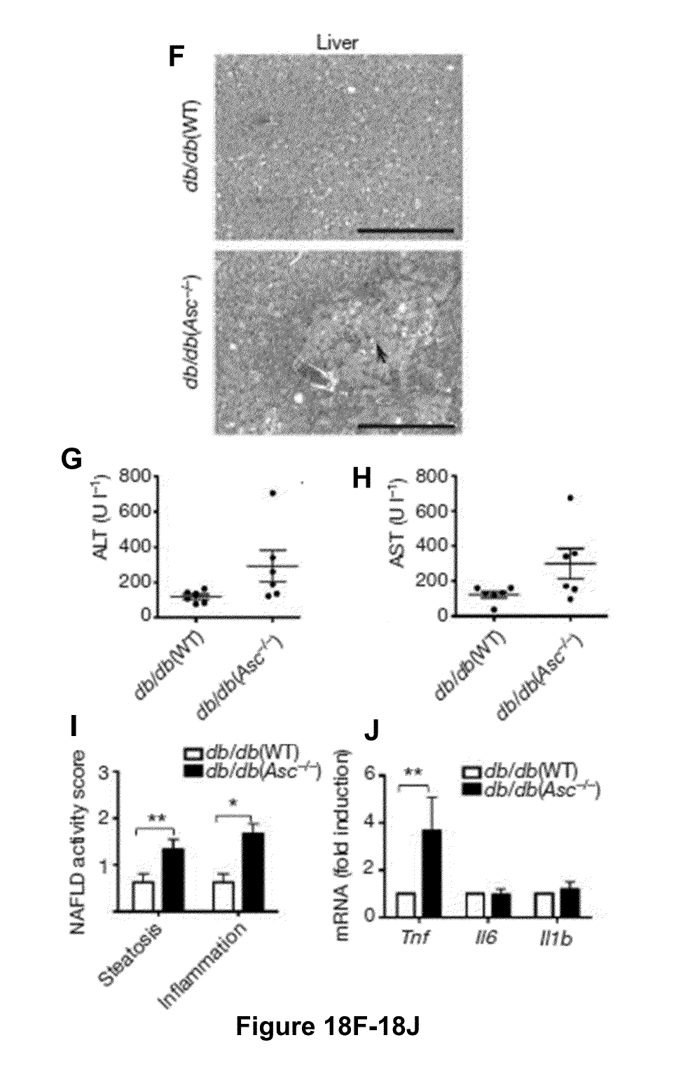

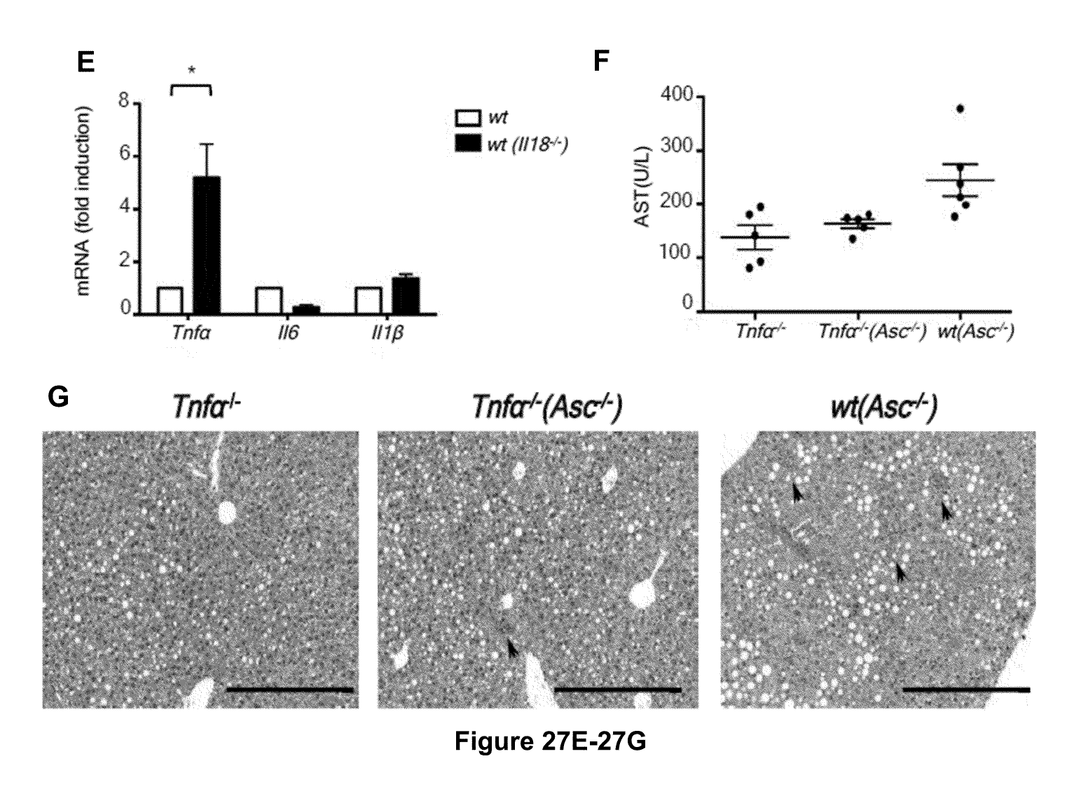

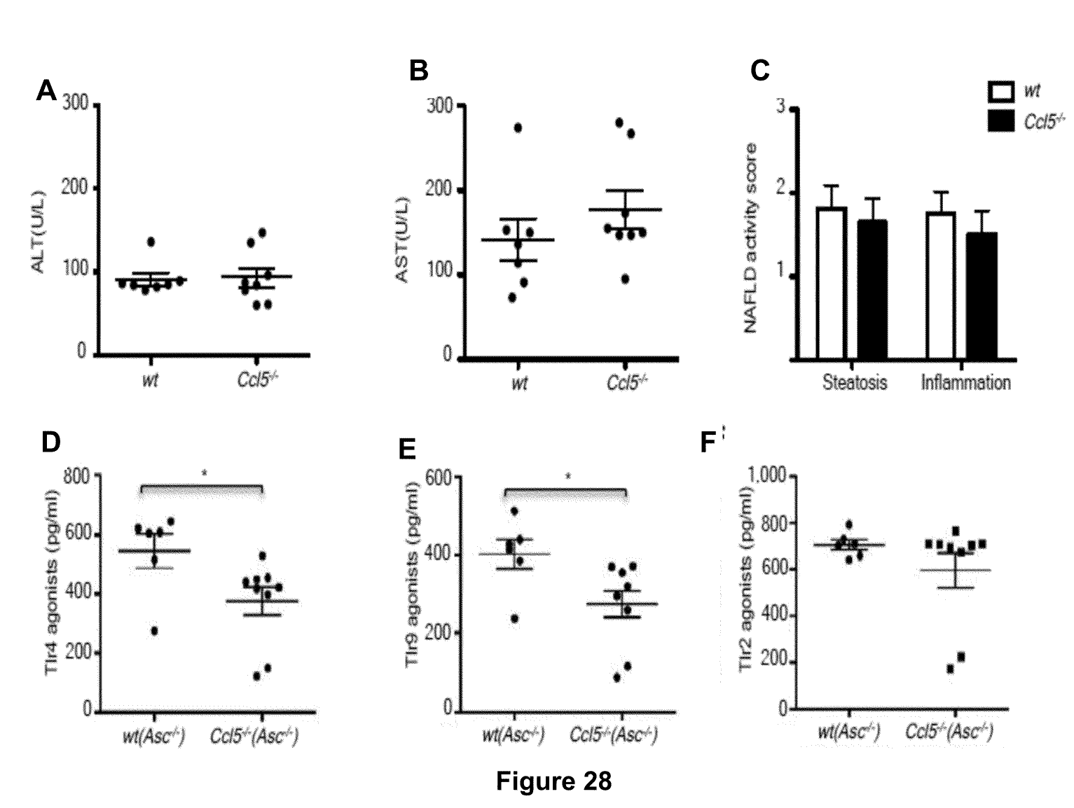

The aberrant gut microbiota in NLRP3 and NLRP6 inflammasome-deficient mice induces colonic inflammation through epithelial induction of CCL5 secretion (Elinav et al., 2011, Cell 145:745-757). To test whether this colon inflammation influences TLR agonist influx into the portal circulation and NASH progression, NASH was induced in wild-type and Ccl5^{-/-} mice that had been either singly housed or co-housed. MCDD-fed, singly housed wild-type and Ccl5^{-/-} mice showed equivalent levels of NASH severity (FIGS. 28A-28C), indicating that CCL5 does not have a role in the early stages of NAFLD/NASH in the absence of the inflammasome-associated colitogenic microbiota. As described elsewhere herein, significantly increased levels of liver injury, inflammation and steatosis in WT(Asc^{-/-}) but not Ccl5^{-/-}(Asc^{-/-}) mice (FIGS. 18A-18C), which led to the conclusion that CCL5 is required for the exacerbation of disease through cohousing with inflammasome-deficient mice. Moreover, Ccl5^{-/-}(Asc^{-/-}) animals showed significantly reduced levels of TLR4 and TLR9 agonists in their portal vein blood than WT(Asc^{-/-}) mice (FIGS. 28D-28F). Together, these results indicated that microbiota-induced subclinical colon inflammation is a determining factor in the rate of TLR agonist influx from the gut, and in NAFLD/NASH progression

The MCDD system is a common model for studying inflammatory processes associated with progression from NAFLD to NASH, yet it lacks many of the associated metabolic phenotypes of NAFLD, such as obesity and insulin resistance (Diehl et al., 2005, Hepatol. Res. 33:138-144). As such, our results in this model might conceivably be limited to the way dysbiosis can influence NASH progression in patients with enhanced intestinal permeability, such as those with inflammatory bowel disease (Broomé et al., 1990, Gut 31:468-472), but not for the majority of patients who suffer from NASH in the context of metabolic syndrome. To test whether alterations in the gut microbiota of inflammasome-deficient mice may affect the rate of progression of NAFLD and other features associated with metabolic syndrome, we extended our studies to genetically obese mice and mice fed with high-fat diet (HFD).

Leptin-receptor deficient (db/db; db is also known as Lepr) animals develop multiple metabolic abnormalities, including NAFLD and impaired intestinal barrier function (Guo et al., 2011, Mucosal Immunol. 4:294-303), that closely resemble the human disease (Ikejima et al., 2005, Hepatol. Res. 33:151-154). However, significant hepatocyte injury, inflammation, and fibrosis are not observed in the absence of a “second hit” (Guebre-Xabier et al., 2000, Hepatology 31:633-640). Upon co-housing of db/db mice with Asc^{-/-} (db/db(Asc^{-/-})) or WT mice (db/db(WT)) for a period of 12 weeks, and as previously shown for Asc^{-/-} mice (Elinav et al., 2011, Cell 145:745-757), the colon and ileum of all db/db(Asc^{-/-}) mice showed mild to moderate mucosal and crypt hyperplasia (FIGS. 18D-18F) that was not seen in db/db(WT) mice.

Strikingly, co-housed db/db(Asc^{-/-}) mice also showed increased levels of hepatocyte injury as evidenced by higher levels of ALT and AST in their sera, and significantly exacerbated steatosis and hepatic inflammation scores when compared with db/db(WT) mice (FIGS. 18G-18I). In addition to a parenchymal inflammatory exudate, patchy areas of markedly degenerated hepatocytes and hepatocytes undergoing necrosis were observed, but only in db/db(Asc^{-/-}) animals (FIG. 18F). Furthermore, some areas of congestion were seen in the centro-lobular zone as well as in the hepatic parenchyma—features that resemble peliosis hepatis, a condition observed in a variety of pathological settings including infection. In accord with our MCDD results, hepatic Tnf mRNA levels were significantly higher in co-housed db/db(Asc^{-/-}) mice than in db/db(WT) animals (FIG. 18J). Again, no significant differences were observed in hepatic IL6 or IL1b mRNA levels (FIG. 18J).

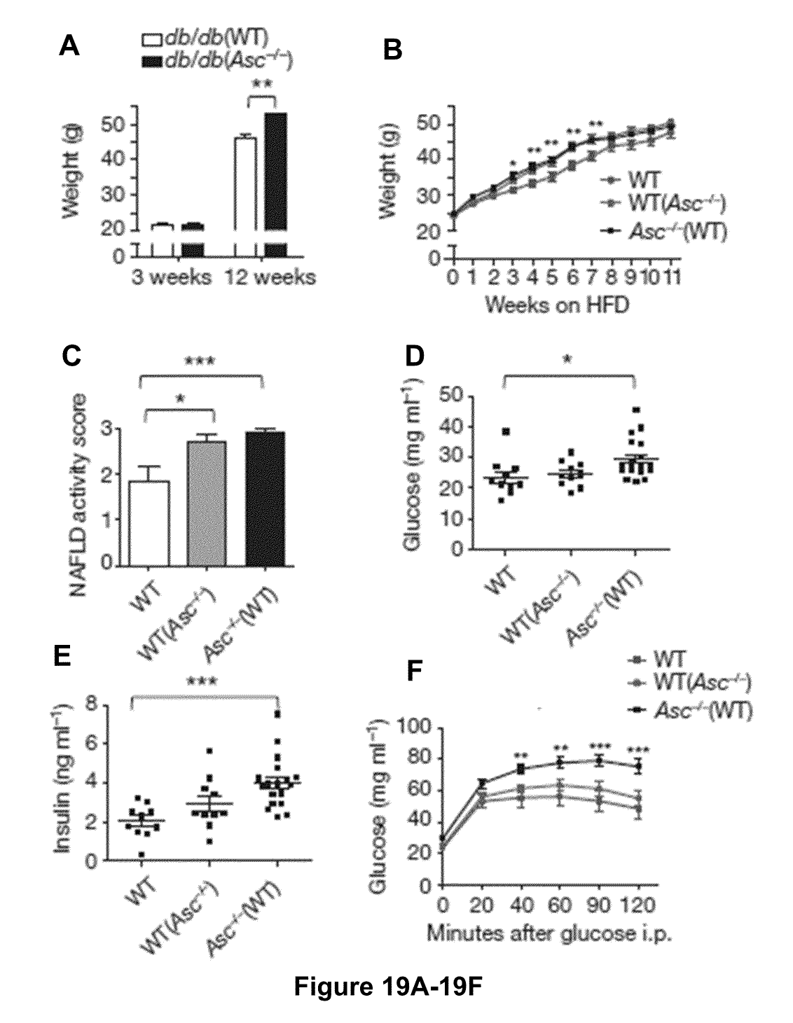

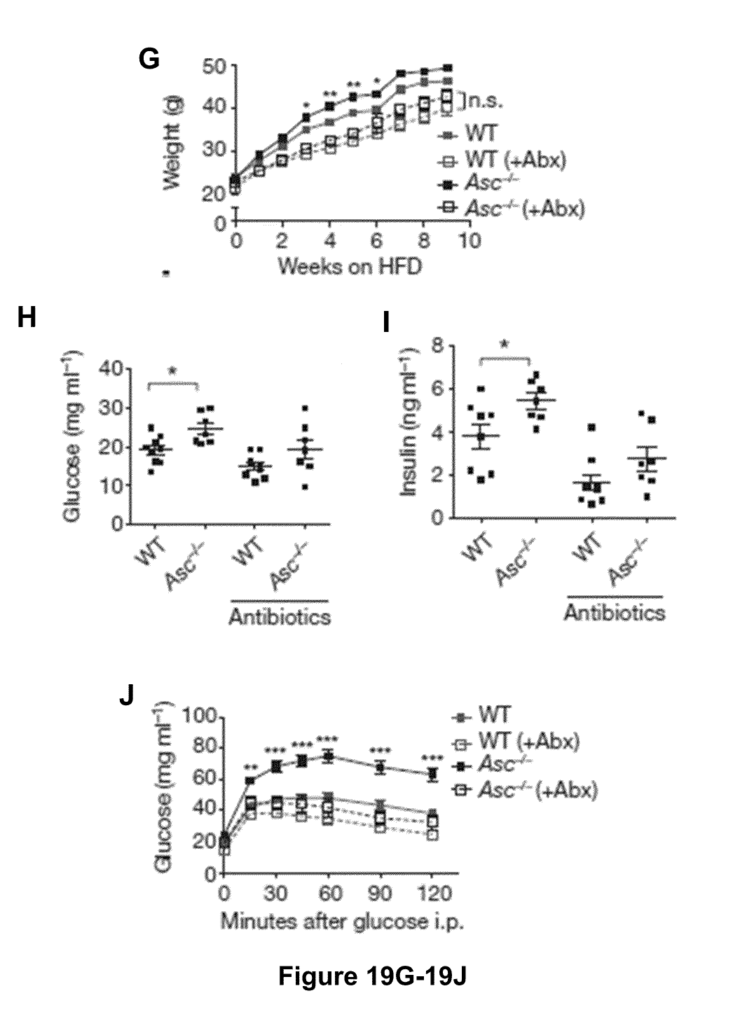

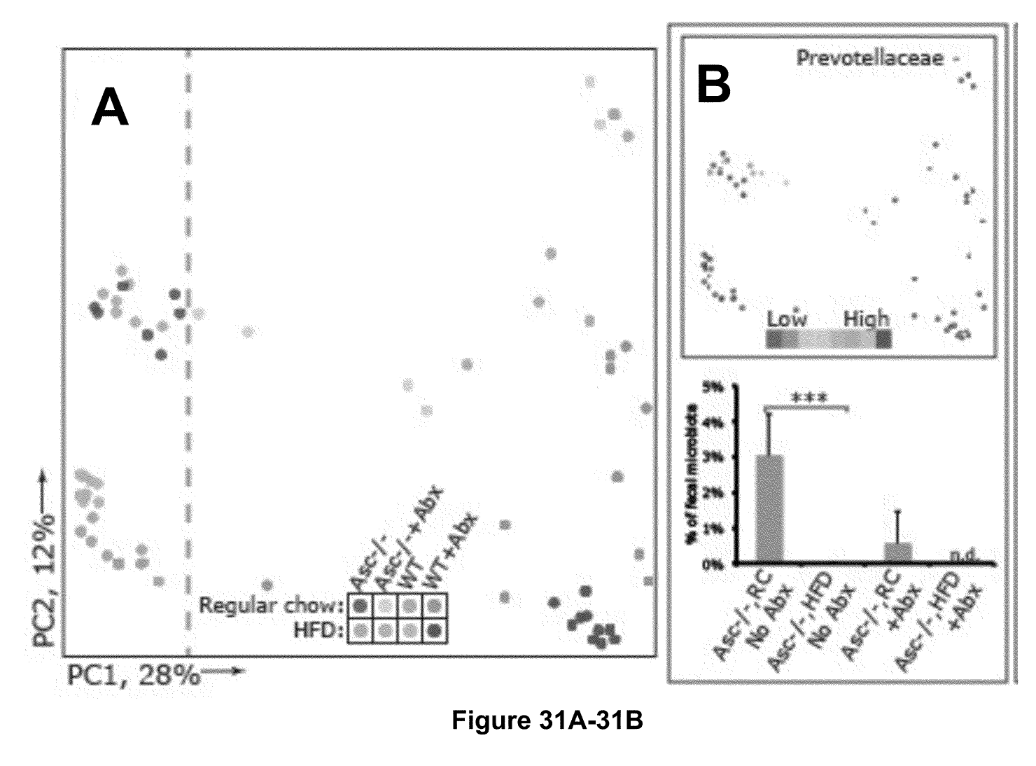

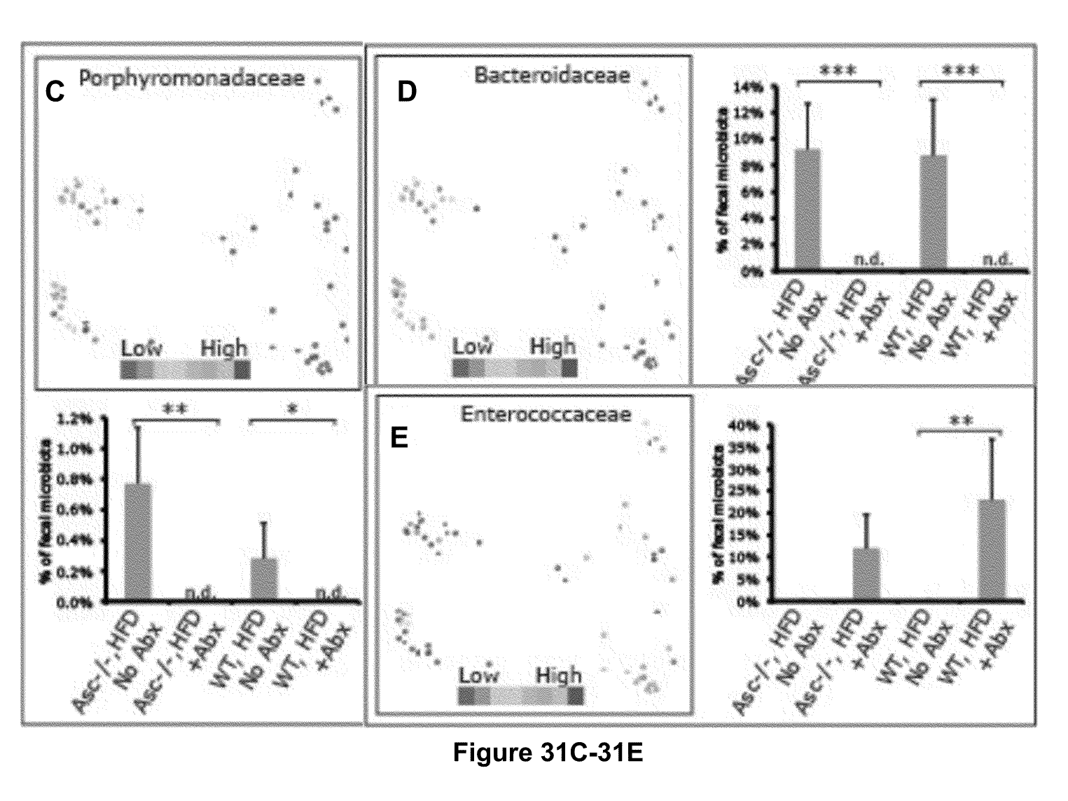

Interestingly, db/db(Asc^{-/-}) mice developed significantly more weight gain compared to db/db(WT) mice after 12 weeks of co-housing (FIG. 19A), indicating that the inflammasome-associated gut microbiota could exacerbate additional processes associated with the metabolic syndrome, such as obesity. To address this possibility, multiple metabolic parameters were monitored in wild-type, WT(Asc^{-/-}) and Asc^{-/-} mice fed a high-fat diet (HFD) for 12 weeks. Strikingly, Asc^{-/-} mice gained body mass more rapidly and featured enhanced hepatic steatosis (FIGS. 19B-19C and FIG. 30F). Asc^{-/-} mice also showed elevated fasting plasma glucose and insulin levels, and decreased glucose tolerance compared to singly housed weight-matched wild-type mice (FIGS. 19D-19F). Interestingly, WT(Asc^{-/-}) mice recapitulated the same increased rate of body mass gain and steatosis when compared to singly housed wild-type controls, although they did not show significant alterations in glucose homeostasis (FIGS. 19D-19F). Nevertheless, antibiotic treatment (ciprofloxacin and metronidazole) abrogated all these abnormalities, including altered rate of gain in body mass, glucose intolerance and fasting plasma insulin levels in Asc^{-/-} mice compared to wild-type mice (FIGS. 19G-19J). Alterations of these metabolic parameters were not caused by changes in feeding behavior between the antibiotic-treated and untreated groups. These results indicate different levels of microbiota-mediated regulation of the various manifestations of the metabolic syndrome: that is, some features (obesity, steatosis) are pronounced and transmissible by co-housing, whereas others (glycaemic control) are affected by alterations in the microbiota but not readily transferable by co-housing. Additionally, a 16S rRNA-based analysis was performed of the faecal microbiota of Asc^{-/-} and wild-type animals that were treated with or without ciprofloxacin and metronidazole (4 weeks) before switching to HFD for 4 additional weeks. Importantly, the analysis demonstrated that Prevotellaceae and Porphyromonadaceae, two family-level taxa, were undetectable in Asc^{-/-} mice 8 weeks after antibiotic treatment (FIGS. 31A-31C, FIG. 33).

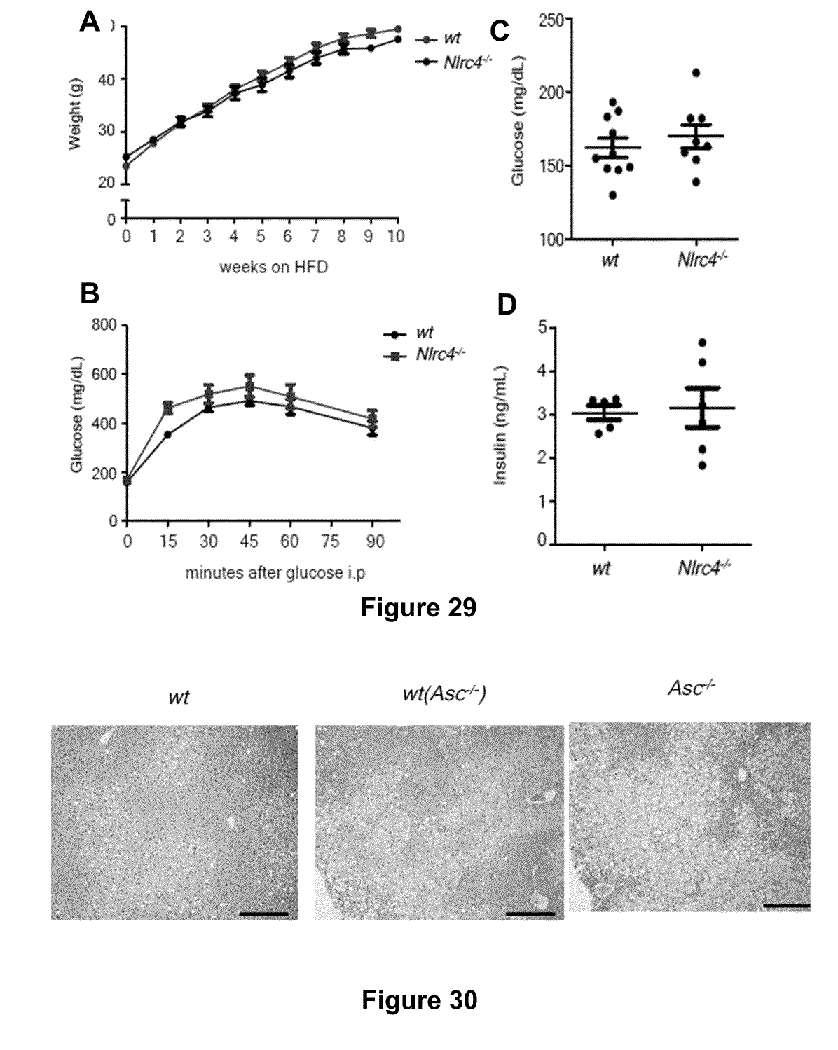

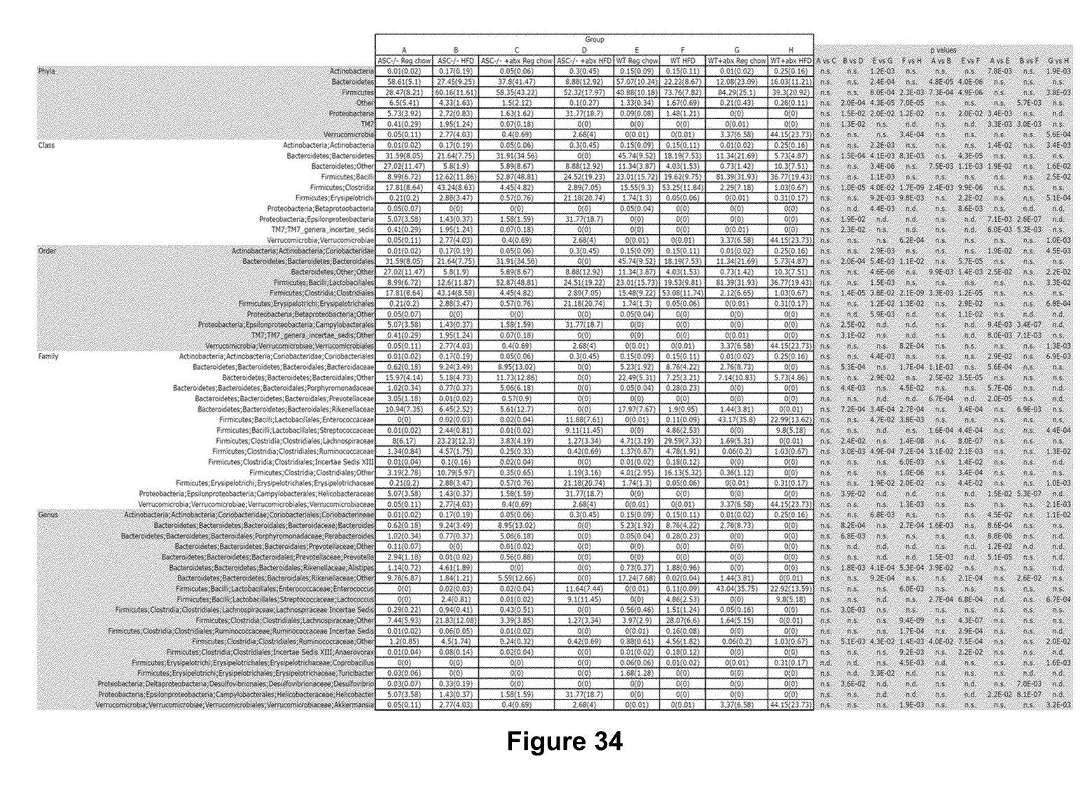

To assess whether these metabolic abnormalities are specific to Asc^{-/-} mice, similar experiments were performed with NhIrc4^{-/-} mice. These mice showed an equal rate of body mass gain, and similar glucose tolerance phenotypes as singly housed wild-type mice, confirming the specificity of the phenotype (FIGS. 29A-29D). 16S rRNA analysis revealed that there was an increased representation of Porphyromonadaceae in Nlrc4^{-/-} mice when compared to wild-type mice (FIG. 34). These results indicate that (1) some metabolic aberrations associated with the dysbiosis of inflammasome-deficient mice can be horizontally transferred from one mouse to another, (2) the gut microbiota of inflammasome-deficient mice has a negative effect on NAFLD progression and glucose homeostasis, and (3) configurational changes in the microbiota, which involve overrepresentation Porphyromonadaceae in combination with alterations in additional taxa, are likely required to produce these host phenotypes.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of diagnosing an altered microbiota associated with an inflammatory disease or disorder in a subject in need thereof, the method comprising the steps of:
a. obtaining a fecal sample from a subject,
b. obtaining bacterial nucleic acid from the fecal sample,
c. amplifying the bacterial nucleic acid using PCR,
d. sequencing the amplicons resulting from the amplification of the bacterial nucleic acid using PCR,
e. identifying the types of bacteria present in the biological sample obtained from the subject by detecting nucleic acid sequences that are specific to particular types of bacteria,
f. quantifying the types of bacteria present in the biological sample obtained from the subject by quantifying nucleic acid sequences that are specific to particular types of bacteria,
g. determining the relative proportions of the types of bacteria present in the fecal sample obtained from the subject,
h. comparing the relative proportions of the types of bacteria present in the fecal sample obtained from the subject with the relative proportions of the types of bacteria present in a normal microbiota,
i. wherein when at least one Lactobacillus spp. is under-represented in the biological sample obtained from the subject, as compared with a normal microbiota, and
ii. wherein when at least one type of bacteria selected from the group consisting of Prevotellaceae, TM7, Porphyromonadaceae, and Erysipelotrichaceae is over-represented in the biological sample obtained from the subject, as compared with a normal microbiota,
i. the subject is diagnosed with an altered microbiota associated with an inflammatory disease or disorder.

2. The method of claim 1, wherein the bacterial nucleic acid is 16S rRNA.

3. The method of claim 1, wherein the inflammatory disease or disorder is at least one inflammatory disease or disorder selected from the group consisting of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH).

4. (canceled)

5. A method of treating an inflammatory disease or disorder associated with an altered microbiota in a subject in need thereof, by modifying the altered microbiota to that of a normal microbiota, the method comprising the steps of:
a. administering to the subject at least one type of bacteria that is under-represented in the altered microbiota of the subject, and
b. administering to the subject at least one antibiotic to diminish the numbers of at least one type of bacteria that is overrepresented in the altered microbiota.

6. The method of claim 5, wherein the at least one type of bacteria that is under-represented in the altered microbiota is at least one Lactobacillus spp.

7. The method of claim 5, wherein at least one Lactobacillus spp. is administered to the subject.

8. The method of claim 5, wherein the at least one type of bacteria that is overrepresented in the altered microbiota is at least one of Prevotellaceae, TM7, Porphyromonadaceae, and Erysipelotrichaceae.

9. (canceled)

10. The method of claim 5, wherein the inflammatory disease or disorder is at least one inflammatory disease or disorder selected from the group consisting of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH).

11. (canceled)

12. A method of treating an inflammatory disease or disorder associated with an altered microbiota in a subject in need thereof, the method comprising: administering to the subject a therapeutically effective amount of a composition comprising a CCL5 inhibitor.

13. The method of claim 12, wherein the CCL5 inhibitor is an antibody that specifically binds to CCL5.

14. (canceled)

15. The method of claim 12, wherein the CCL5 inhibitor is an antisense nucleic acid.

16. (canceled)

17. The method of claim 12, wherein the CCL5 inhibitor is at least one selected from the group consisting of: a chemical compound, a protein, a peptide, a peptidomemetic, a ribozyme, and a small molecule chemical compound.

18. The method of claim 12, wherein the inflammatory disease or disorder is at least one inflammatory disease or disorder selected from the group consisting of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH).

19. (canceled)

20. A method of treating an inflammatory disease or disorder associated with an altered microbiota in a subject in need thereof, the method comprising:
administering to the subject a therapeutically effective amount of a composition comprising a CCL5 receptor inhibitor.

21. The method of claim 20, wherein the CCL5 receptor is at least one selected from the group consisting of: CCR1, CCR3, CCR4, CCR5 and GPR75.

22. The method of claim 20, wherein the CCL5 receptor inhibitor is an antibody that specifically binds to a CCL5 receptor.

23. (canceled)

24. The method of claim 20, wherein the CCL5 receptor inhibitor is an antisense nucleic acid.

25. (canceled)

26. The method of claim 20, wherein the CCL5 receptor inhibitor is at least one selected from the group consisting of: a chemical compound, a protein, a peptide, a peptidomemetic, a ribozyme, and a small molecule chemical compound.

27. The method of claim 20, wherein the inflammatory disease or disorder is at least one inflammatory disease or disorder selected from the group consisting of: inflammatory bowel disease, celiac disease, colitis, intestinal hyperplasia, metabolic syndrome, obesity, rheumatoid arthritis, liver disease, hepatic steatosis, fatty liver disease, non-alcoholic fatty liver disease (NAFLD), and non-alcoholic steatohepatitis (NASH).

28. (canceled)
